SEQUENCE LISTING (18 sequences)

SEQ ID NO: 1 - protein, 11 aa, Artificial Sequence
  ITIM motif
  misc_feature: Synthetic
KEKLCYVALDF

SEQ ID NO: 2 - protein, 11 aa, Artificial Sequence
  mutant actin peptide
  misc_feature: Synthetic
KEKLCFVALDF

SEQ ID NO: 3 - protein, 10 aa, Artificial Sequence
  irrelevant control peptide
  misc_feature: Synthetic
EYQKASGVSG

SEQ ID NO: 4 - DNA, 1128 bp, Homo sapiens
  misc_feature: actin cDNA NM_001101.4
atggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctag

SEQ ID NO: 5 - protein, 375 aa, Homo sapiens
  MISC_FEATURE: actin protein, NP_001092.1
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 6 - DNA, 1788 bp, Homo sapiens
  misc_feature: PTPN6 cDNA, transcript variant 1 NM_002831.5
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 7 - protein, 595 aa, Homo sapiens
  MISC_FEATURE: tyrosine-protein phosphatase non-receptor type 6 isoform 1 [Homo sapiens] NP_002822.2
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 8 - protein, 597 aa, Homo sapiens
  MISC_FEATURE: tyrosine-protein phosphatase non-receptor type 6 isoform 2 [Homo sapiens] NP_536858.1
MLSRGWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 9 - protein, 624 aa, Homo sapiens
  misc_feature: tyrosine-protein phosphatase non-receptor type 6 isoform 3 [Homo sapiens] NP_536859.1
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKSLESSAGTVAASPVRRGGQRGLPVPGPPVLSPDLHQLPVLAPLHPAADTRRMCMRTCTLRTRGRRK

SEQ ID NO: 10 - DNA, 1782 bp, Homo sapiens
  misc_feature: PTPN11, transcript variant 1 cDNA NM_002834.4
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatga

SEQ ID NO: 11 - protein, 593 aa, Homo sapiens [sequence omitted]

SEQ ID NO: 12 - protein, 460 aa, Homo sapiens
  MISC_FEATURE: tyrosine-protein phosphatase non-receptor type 11 isoform 2 [Homo sapiens] NP_542168.1
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR

SEQ ID NO: 13 - DNA, 1794 bp, Homo sapiens
  misc_feature: PTPN6 transcript variant 2, cDNA NM_080548.4
atgctgtcccgtgggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtga

SEQ ID NO: 14 - DNA, 1875 bp, Homo sapiens
  misc_feature: PTPN6 transcript variant 3, cDNA NM_080549.3
atggtgaggtggtttcaccgagacctcagtgggctggatgcagagaccctgctcaagggccgaggtgtccacggtagcttcctggctcggcccagtcgcaagaaccagggtgacttctcgctctccgtcagggtgggggatcaggtgacccatattcggatccagaactcaggggatttctatgacctgtatggaggggagaagtttgcgactctgacagagctggtggagtactacactcagcagcagggtgtcctgcaggaccgcgacggcaccatcatccacctcaagtacccgctgaactgctccgatcccactagtgagaggtggtaccatggccacatgtctggcgggcaggcagagacgctgctgcaggccaagggcgagccctggacgtttcttgtgcgtgagagcctcagccagcctggagacttcgtgctttctgtgctcagtgaccagcccaaggctggcccaggctccccgctcagggtcacccacatcaaggtcatgtgcgagggtggacgctacacagtgggtggtttggagaccttcgacagcctcacggacctggtggagcatttcaagaagacggggattgaggaggcctcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaagagcttggagtctagtgcagggaccgtggctgcgtcacctgtgagacggggtggccagaggggactgccagtgccgggtccccctgtgctgtctcctgacctgcaccaactgcctgtacttgcccccctgcacccggctgcagacacaaggaggatgtgtatgagaacctgcacactaagaacaagagggaggagaaagtga

SEQ ID NO: 15 - DNA, 1383 bp, Homo sapiens
  misc_feature: PTPN11 transcript variant 2, cDNA NM_080601.2
atgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcaggtga

SEQ ID NO: 16 - protein, 375 aa, Artificial Sequence
  Actin cytoplasmic 1
  misc_feature: Synthetic
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMRKEITALAPSTMKIKIIAPPERKYSVWIGGSILASLSTFQQMWISKQEYDESGPSIVHRKCF

SEQ ID NO: 17 - DNA, 25 bp, Artificial Sequence
  sgRNA sequence top
  misc_feature: Synthetic
caccgtcggcccagtcgcaagaacc

SEQ ID NO: 18 - DNA, 25 bp, Artificial Sequence
  sgRNA sequence bottom
  misc_feature: Synthetic
aaacggttcttgcgactgggccgac

(19) United States
(12) Patent Application Publication
(10) Pub. No.: US 2019/0343801 A1
(43) Pub. Date: Nov. 14, 2019

(54) COMPOSITIONS AND METHODS FOR CONTROLLING NATURAL KILLER CELL ACTIVATION AND FUNCTION

(71) Applicant: BAR ILAN UNIVERSITY, Ramat Gan (IL)

(72) Inventor: Mira BARDA-SAAD, Ganney Tikva (IL)

(21) Appl. No.: 16/478,552

(22) PCT Filed: Jan. 17, 2018
(86) PCT No.: PCT/IL2018/050064

§ 371 Date: Jul. 17, 2019

Related U.S. Application Data
(60) Provisional application No. 62/446,899, filed on Jan. 17, 2017.

(51) Int. Cl. A61K 31/4015; A61K 31/025; A61K 31/404; A61K 31/4409; G01N 33/50

(52) U.S. Cl. CPC A61K 31/4015; A61K 31/025; A61K 31/404; G01N 2333/4712; G01N 33/5035; G01N 33/5061; A61K 31/4409

(57) ABSTRACT

The present invention provides means to affect the dynamics of actomyosin network in natural killer (NK) cells, and thereby to confer selective control on killing efficiencies of NK cell populations. Compositions and methods of the present invention, particularly those using small molecules, provide a powerful tool for controlling NK cell activation and function in various conditions, in health and disease, most notably in viral infections, autoimmunity, immunodeficiency, GVHD and cancer.

### FIELD OF THE INVENTION

The present invention pertains to the field of molecular immunology, and specifically immunology of the hematopoietic cells. More specifically, the present invention provides specific compounds targeted at modulation of actin and/or myosin network dynamics in hematopoietic cells, specifically lymphocytes such as NK cells, thereby conferring selective control on killing efficiencies of NK cell populations.

### BACKGROUND REFERENCES

1. Vivier, E., Nunes, J. A. & Vely, F. Natural killer cell signaling pathways. Science 306, 1517-1519 (2004).
2. Chhabra, E. S. & Higgs, H. N. The many faces of actin: matching assembly factors with cellular structures. Nat Cell Biol 9, 1110-1121 (2007).
3. Pollard, T. D. & Borisy, G. G. Cellular motility driven by assembly and disassembly of actin filaments. Cell 112, 453-465 (2003).
4. Treanor, B. et al. The membrane skeleton controls diffusion dynamics and signaling through the B cell receptor. Immunity 32, 187-199 (2010).
5. Long, E. O. Negative signaling by inhibitory receptors: the NK cell paradigm. Immunological reviews 224, 70-84 (2008).
6. Lanier, L. L. Up on the tightrope: natural killer cell activation and inhibition. Nature immunology 9, 495-502 (2008).
7. Stebbins, C. C. et al. Vav 1 dephosphorylation by the tyrosine phosphatase SHP-1 as a mechanism for inhibition of cellular cytotoxicity. Mol Cell Biol 23, 6291-6299 (2003).
8. Babich, A. et al. F-actin polymerization and retrograde flow drive sustained PLCgamma1 signaling during T cell activation. The Journal of cell biology 197, 775-787 (2012).
9. Yi, J., Wu, X. S., Crites, T. & Hammer, J. A., 3rd Actin retrograde flow and actomyosin II arc contraction drive receptor cluster dynamics at the immunological synapse in Jurkat T cells. Molecular biology of the cell 23, 834-852 (2012).
10. Braiman, A., Barda-Saad, M., Sommers, C. L. & Samelson, L. E. Recruitment and activation of PLCgamma1 in T cells: a new insight into old domains. Embo J 25, 774-784 (2006).
11. Watanabe, D. et al. Four tyrosine residues in phospholipase C-gamma 2, identified as Btk-dependent phosphorylation sites, are required for B cell antigen receptor-coupled calcium signaling. The Journal of biological chemistry 276, 38595-38601 (2001).
12. Zacharias, D. A., Violin, J. D., Newton, A. C. & Tsien, R. Y. Partitioning of lipid-modified monomeric GFPs into membrane microdomains of live cells. Science 296, 913-916 (2002).
13. Lorenz, U. Protein tyrosine phosphatase assays. Current protocols in immunology/edited by John E. Coligan . . . [et al.] Chapter 11, Unit 11 17 (2011).
14. Pauker, M. H. et al. WASp Family Verprolin-homologous Protein-2 (WAVE2) and Wiskott-Aldrich Syndrome Protein (WASp) Engage in Distinct Downstream Signaling Interactions at the T Cell Antigen Receptor Site. The Journal of biological chemistry 289, 34503-34519 (2014).

### BACKGROUND OF THE INVENTION

Natural killer (NK) cells are lymphocytes of the innate immune system which play an important role in providing immunological surveillance and defense mechanisms in multiple processes, including tumor growth, viral infections, autoimmunity, and graft-versus-host disease. The cellular and molecular mechanisms responsible for regulation of NK cell activation or tolerance, involve a careful balance between activating and inhibitory signals initiated upon the engagement of NK cells with a variety of activating and inhibitory receptors [1]. Despite numerous studies in this field, the mechanisms dictating NK cell activation and regulating their functional outcome are still poorly understood.

The actin-myosin cytoskeleton has been identified as one of the crucial factors in mechanisms underlying cellular immunity, and in NK cell activation in particular. Lymphocyte-mediated immunity involves extensive lymphocyte trafficking in the bloodstream and tissues, and their accumulation at the inflammation sites. These processes are facilitated by the formation of various actin structures and by the activity of myosin motors providing mechanical foundation for lytic granule secretion, motility, adhesion, and tissue invasion. It has been demonstrated that the diversity and flexibility of these cellular functions is contingent on rapid assembly of filamentous actin (F-actin) and on contractile forces generated by the myosin network [2-4]. According to the presently accepted notion, the actin network provides the structural basis for formation of immunological synapse (IS), i.e. the lymphocyte-target-cell conjugate site, and for integration of molecular complexes and signaling effectors. Physical forces generated by the actin-myosin (actomyosin) network are further responsible for the process of mechanotransduction, i.e. the conversion of mechanical forces into chemical signals, whereby the “pushing” force generated by actin polymerization and the “pulling” force of myosin are translated into signaling cascades. However, there are still open questions as to the molecular underpinning of mechanotransduction, and how this process leads to control on the NK cell activation/inhibition.

It has been known that the NK cell activation occurs upon interaction of NK cells with a potential target, and ligation of NK activation receptors to ligands on target cells resulting in activation of protein tyrosine kinase (PTK)-dependent signaling pathways. If inappropriately activated, the NK cells have the potential to cause autoimmune damage. To prevent such inappropriate activation or inhibition, the NK cells possess sophisticated mechanisms that integrate signals from both activating and inhibitory receptors. The mechanism of NK cells inhibition has received increasing attention in a number of studies. One of the mechanisms to prevent NK cell activation, is the engagement of NK cell inhibitory receptors by self-major histocompatibility complex (MHC) class I molecules expressed on healthy autologous cells that transduces inhibitory signals. More specifically, the NK cells inhibition is controlled by ligation of the inhibitory receptors, including members of the killer-cell immunoglobulin-like receptors (KIR) and the CD94-NKG2A receptor, to MHC class I molecules. This engagement antagonizes activating pathways by recruiting the protein tyrosine phosphatase (PTP) Src homology region 2 domain-containing phosphatase-1 (SHP-1) to the NK immunological synapse (NKIS), i.e. the NK/target interaction site [5, 6]. SHP-1 dephosphorylates key signaling molecules, such as VAV1, thereby blocking NK cell activation [7].

Notwithstanding the above, the actomyosin cytoskeleton in NK cells was regarded merely as a static platform, and studies mainly focused on activating and inhibitory mechanisms that control actin rearrangement. However, increasing evidence strongly indicates that a dynamic actomyosin network, rather than a static one, is crucial for regulating cellular responses. Moreover, thus far, actomyosin movement or retrograde flow (ARF), and its spatial-temporal dynamics, have been never explored in the context of NK cell effector function, and as possible effectors or controllers of the cellular immune response.

Thus, there is an unmet need in the art for effective modulators of lymphocyte activation, specifically, modulators for NK cell activation. These need are addressed by the present invention that provides modulators of the ARF and uses thereof as modulators of lymphocyte activation.

### SUMMARY OF THE INVENTION

In a first aspect, the present invention relates to a modulator that modulates at least one of actin and myosin retrograde flow (ARF) in a cell, and uses thereof in methods for modulating hematopoietic cell activation. In some embodiments, the hematopoietic cell is a lymphocyte cell forming an activating or inhibitory immunological synapse (IS). Still further, the invention provides any of the modulators disclosed by the invention for use in modulating hematopoietic cell activation in a subject in need thereof. Another aspect of the invention relates to a therapeutic effective amount of any one of the modulators described by the invention, for use in a method of treating, preventing, inhibiting, reducing, eliminating, protecting or delaying the onset of an immune-related disorder, specifically, any one of tumor, malignancy, viral infections or graft versus host disease in a subject in need thereof.

It is another important aspect of the present invention to provide a composition comprising an effective amount of the modulator of the invention or any vehicle, matrix, nano- or micro-particle comprising the same for use in modulation of hematopoietic cell activation. More specifically, in some embodiments the hematopoietic cell may be a lymphocyte and the modulator of the invention is characterized in that it modulates at least one of ARF and actomyosin dynamics in a lymphocyte cell forming an activating or inhibitory IS.

A further important aspect of the present invention relates to a method for modulating hematopoietic cell activation, specifically, lymphocyte cells activation. More specifically, the method of the invention may comprise contacting the cell with a modulatory effective amount of a modulator that modulates at least one of actin and myosin ARF in a cell, or with any vehicle, matrix, nano- or micro-particle, or composition comprising the same.

A further aspect of the invention relates to a method for treating, preventing, inhibiting, reducing, eliminating, protecting or delaying the onset of an immune-related disorder in a subject in need thereof. In some embodiments, the method comprising administering to the treated subject a therapeutically effective amount of at least one modulator that modulates at least one of actin and myosin ARF in a cell, or of any vehicle, matrix, nano- or micro-particle, or composition comprising the same. In some specific embodiments, such cell may be a lymphocyte cell forming an activating or inhibitory IS.

The invention further provides in another aspect thereof a modulator of hematopoietic cell activation, or any vehicle, matrix, nano- or micro-particle, or composition comprising the same. In some specific embodiments, such modulator is characterized in that it modulates at least one of actin and myosin ARF in a cell forming.

In yet another aspect, the invention relates to a method for screening for a modulator of a NK cell activation.

These and other aspects of the invention will become apparent as the description proceeds.

### BRIEF DESCRIPTION OF FIGURES

FIG. 1A-1G. Analysis of SHP-1:β-actin complex formation following NK cell inhibition

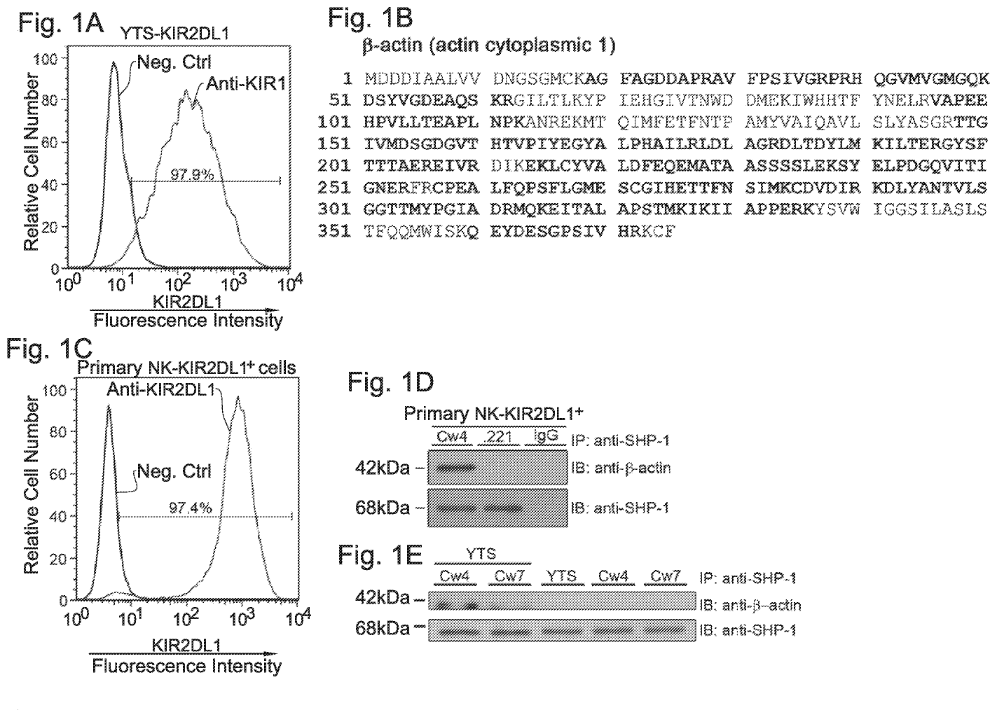

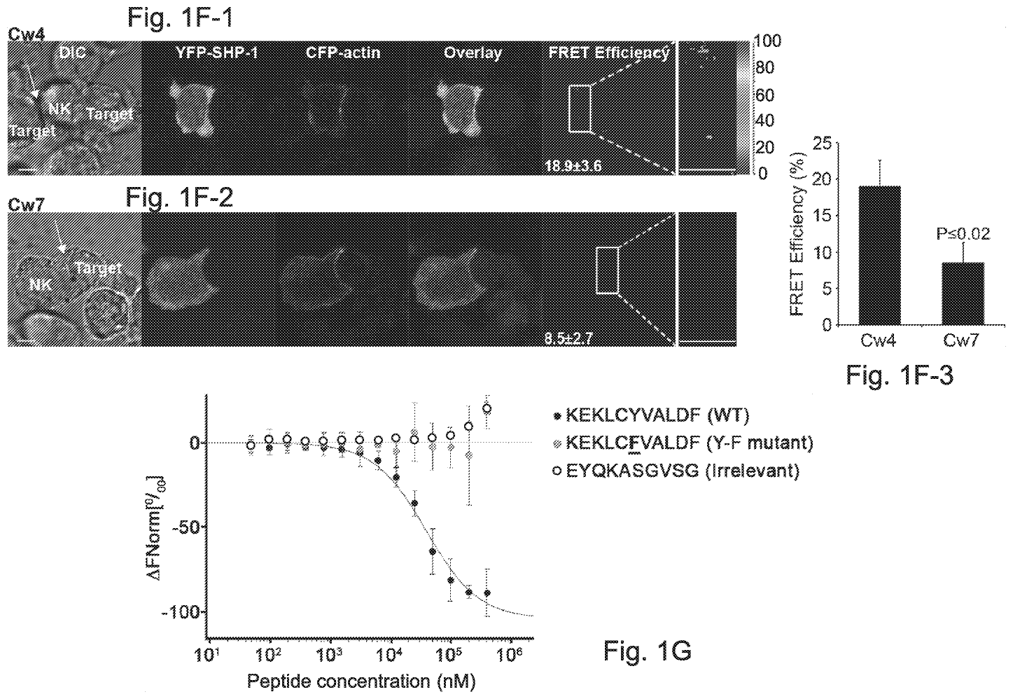

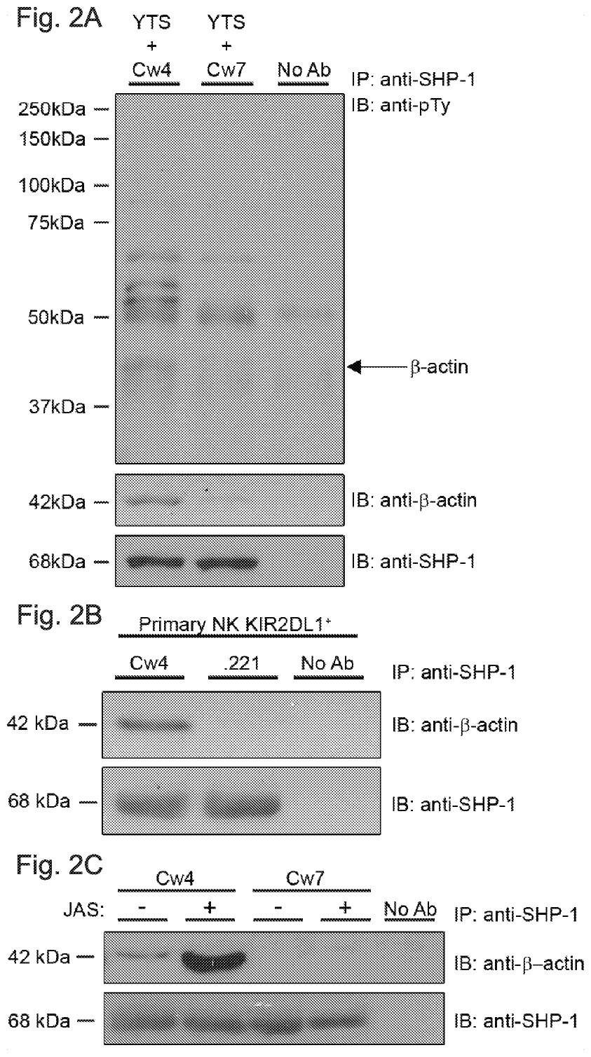

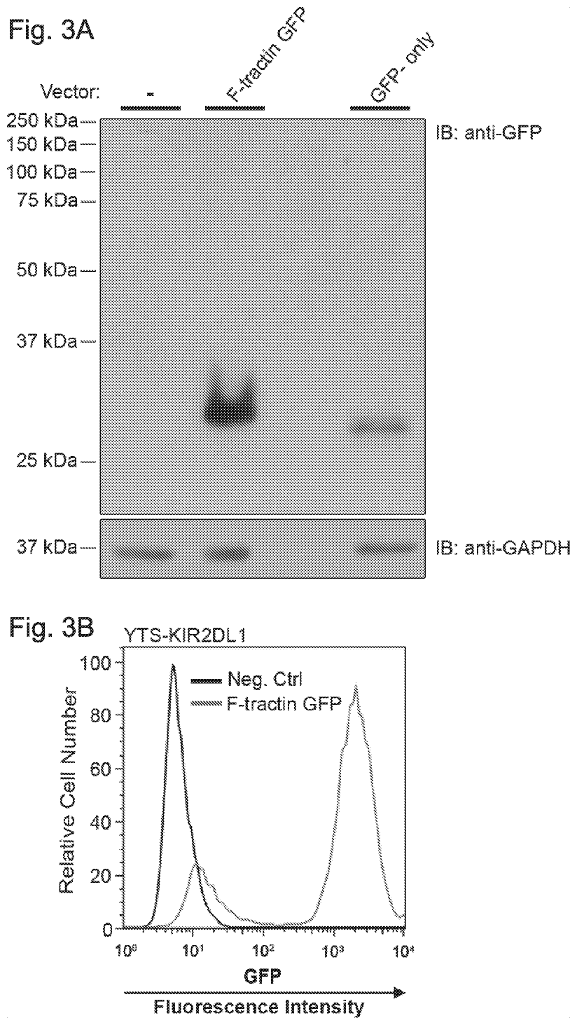

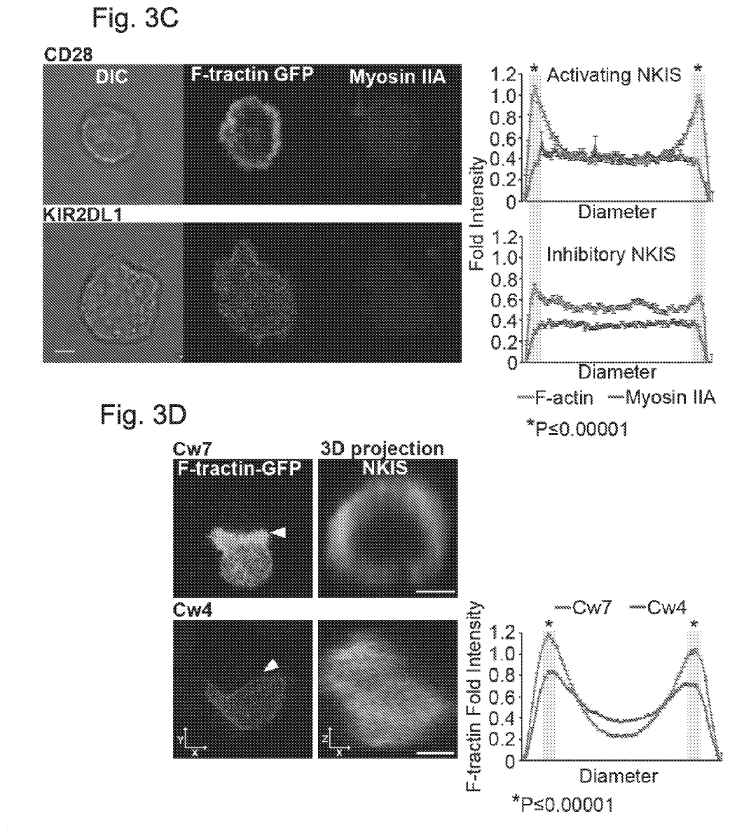

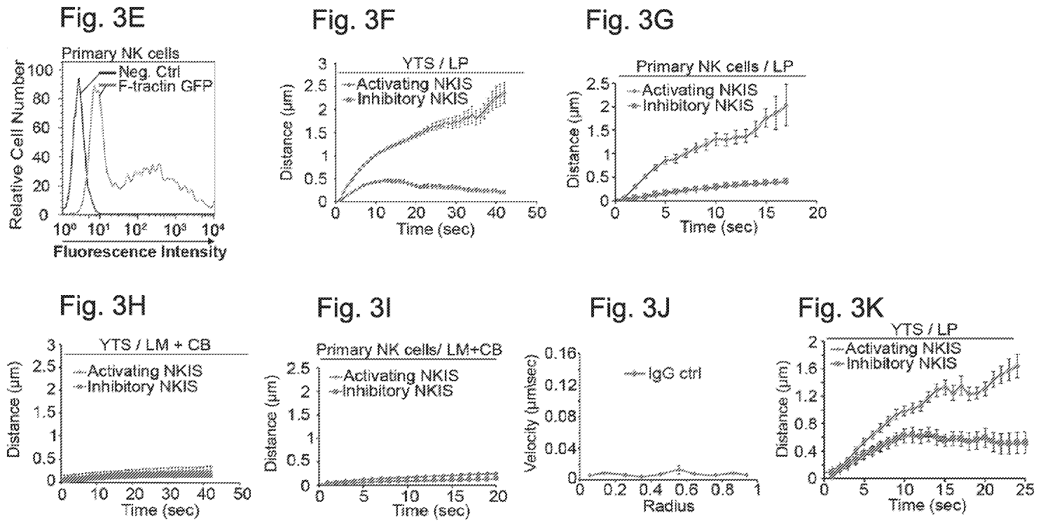

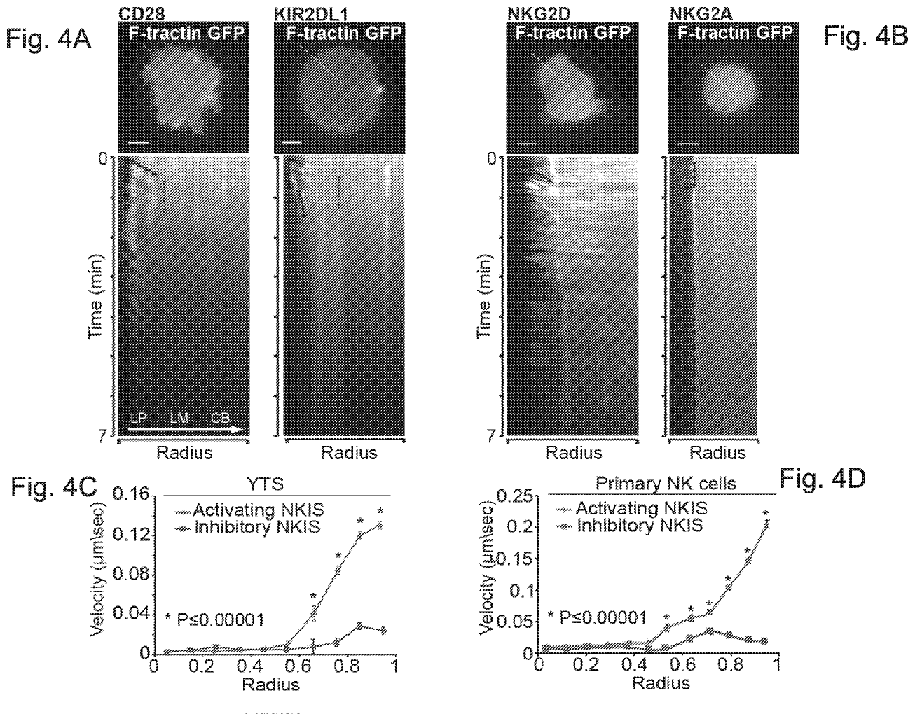

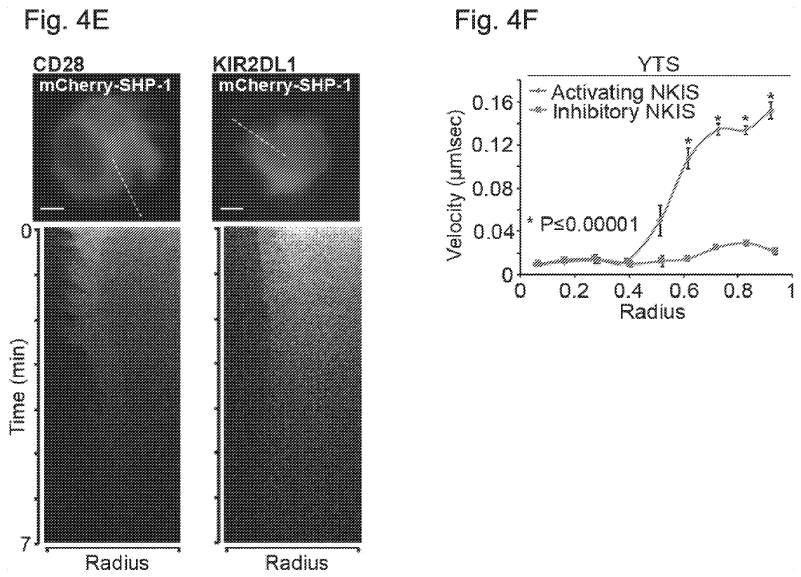

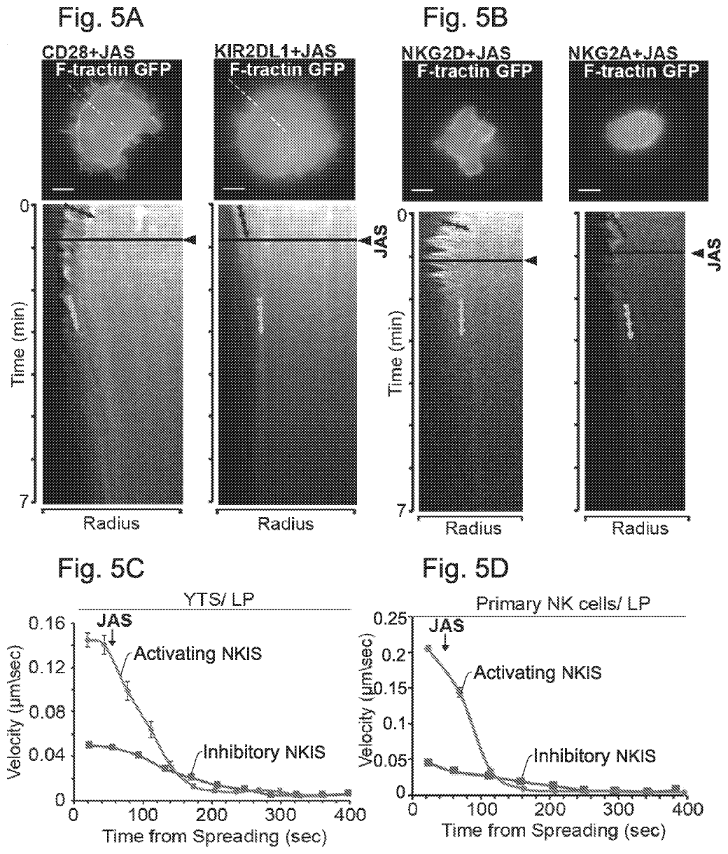

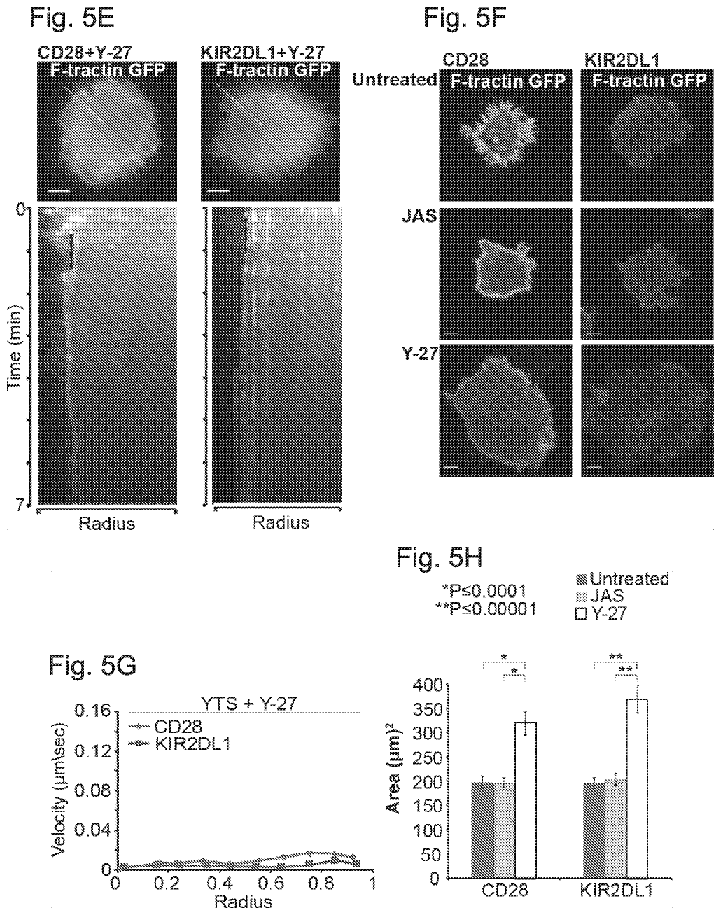

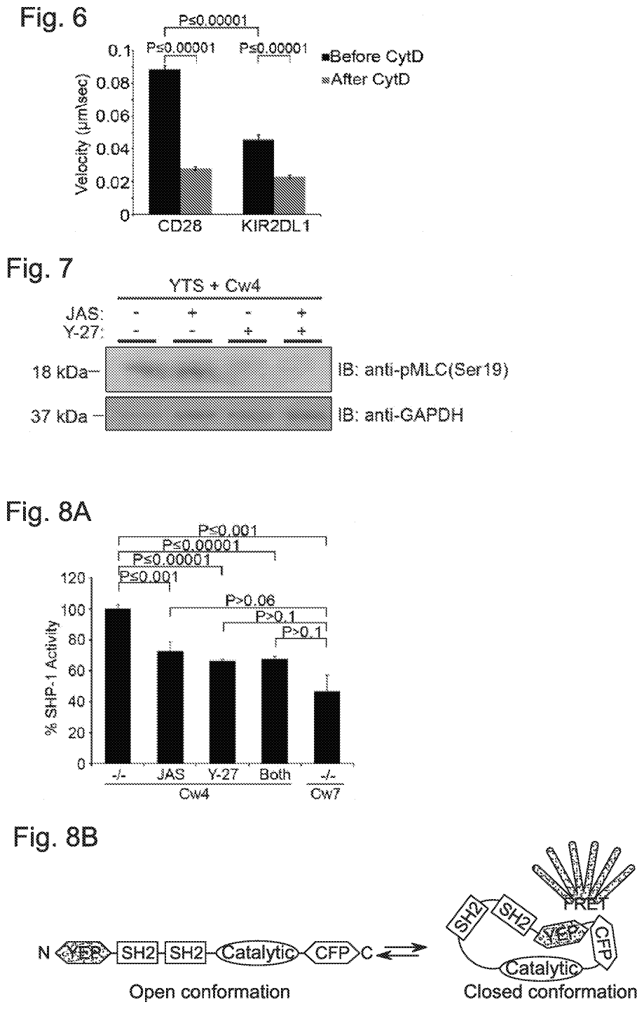

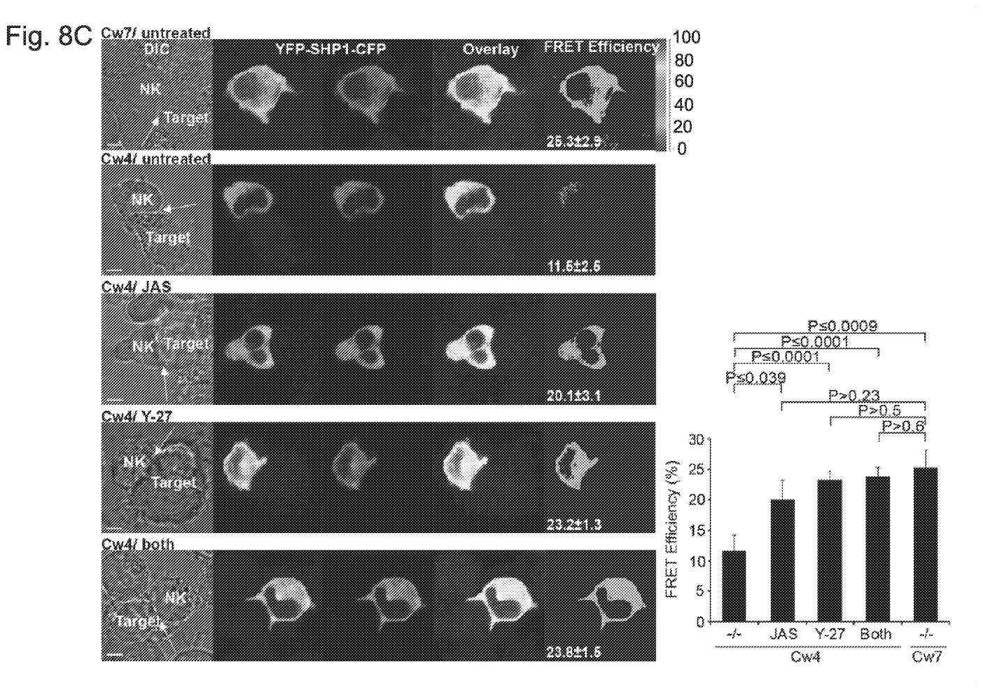

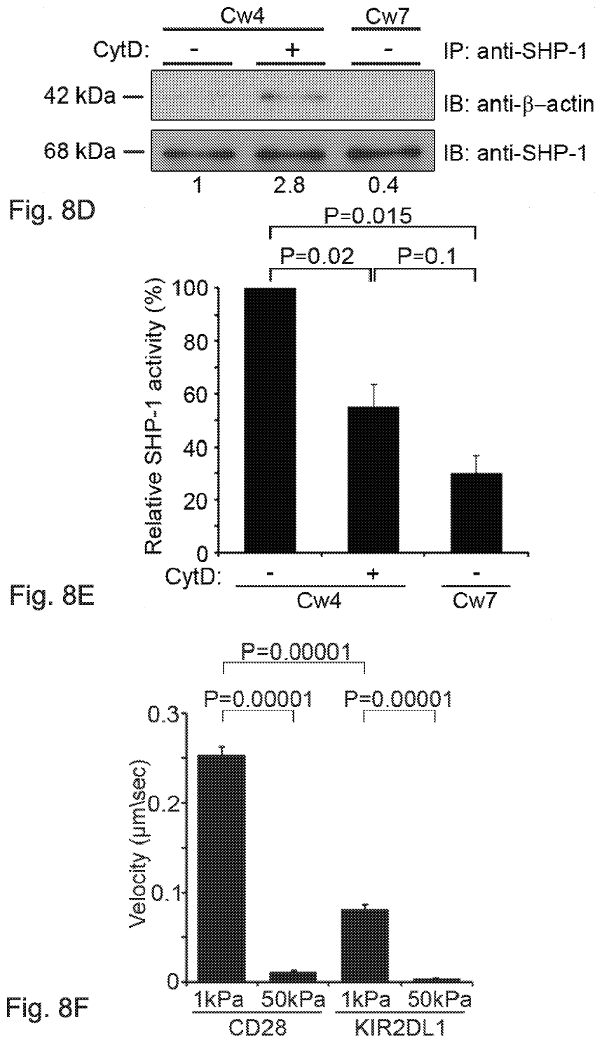

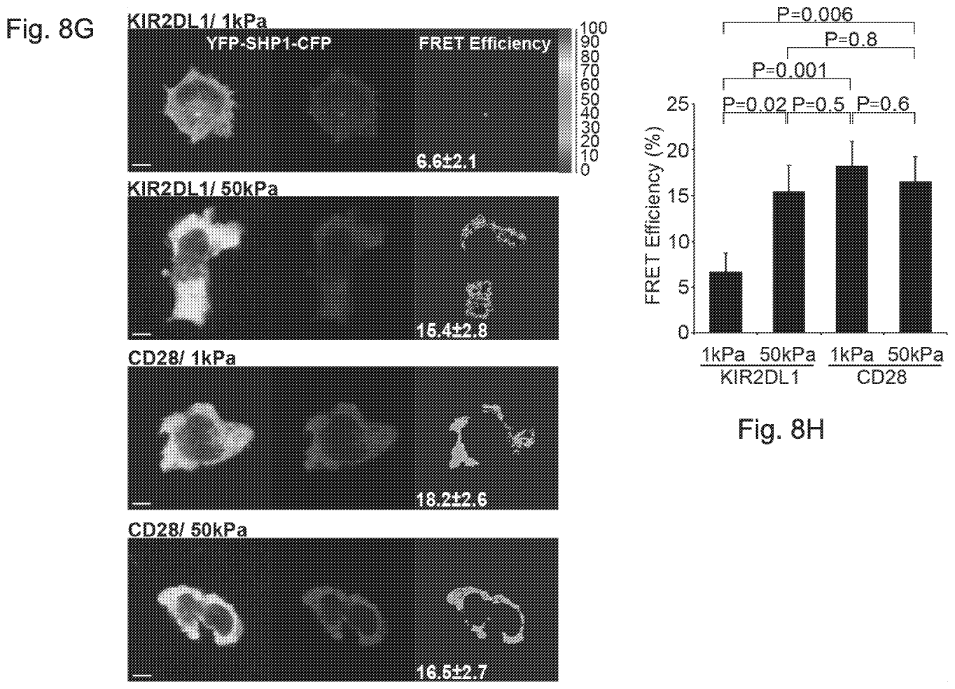

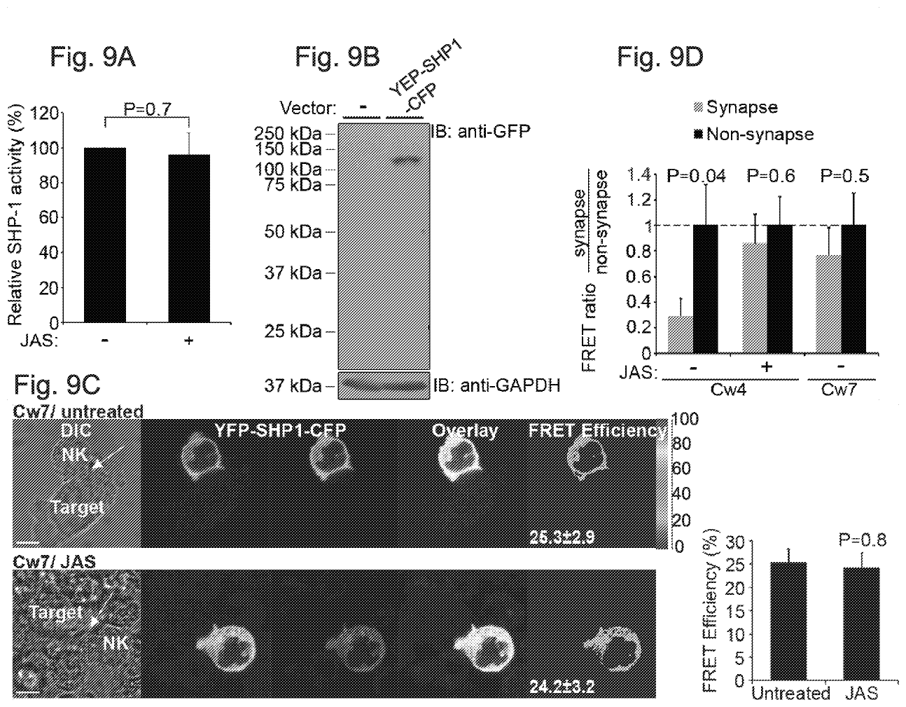

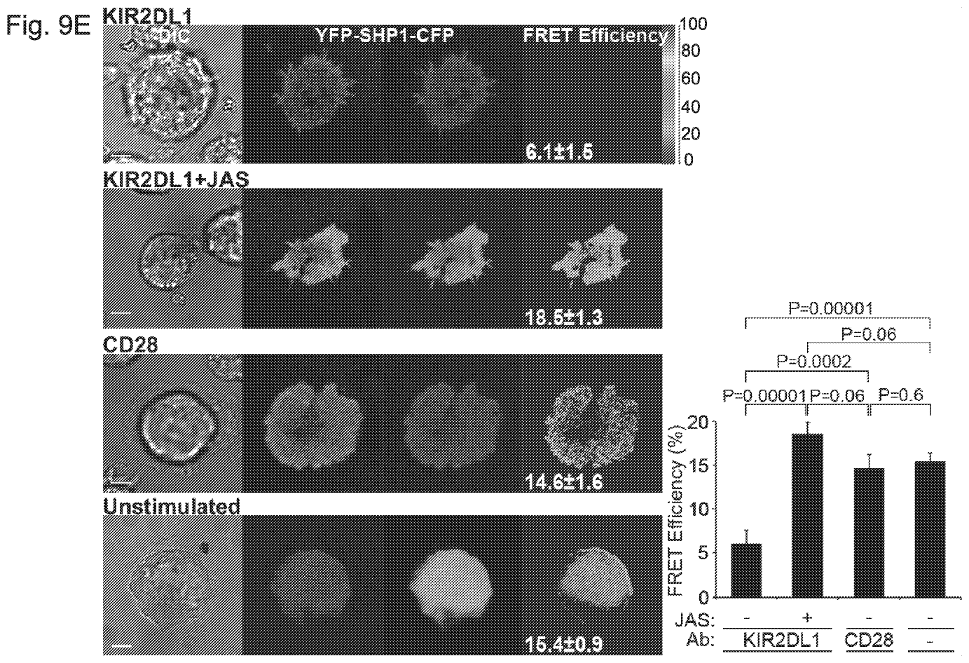

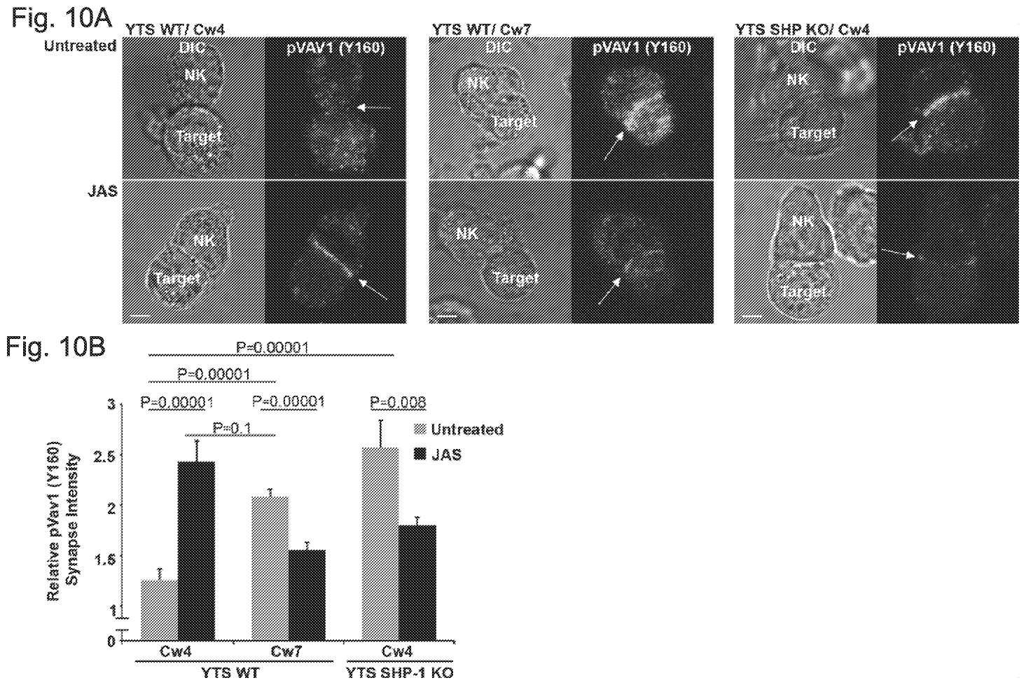

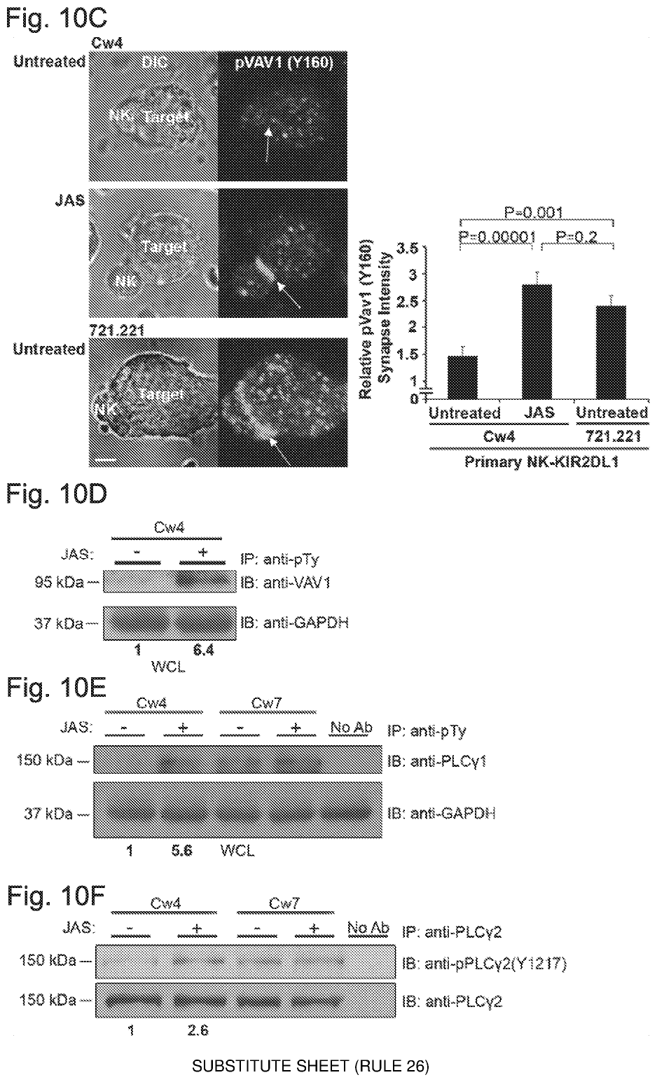

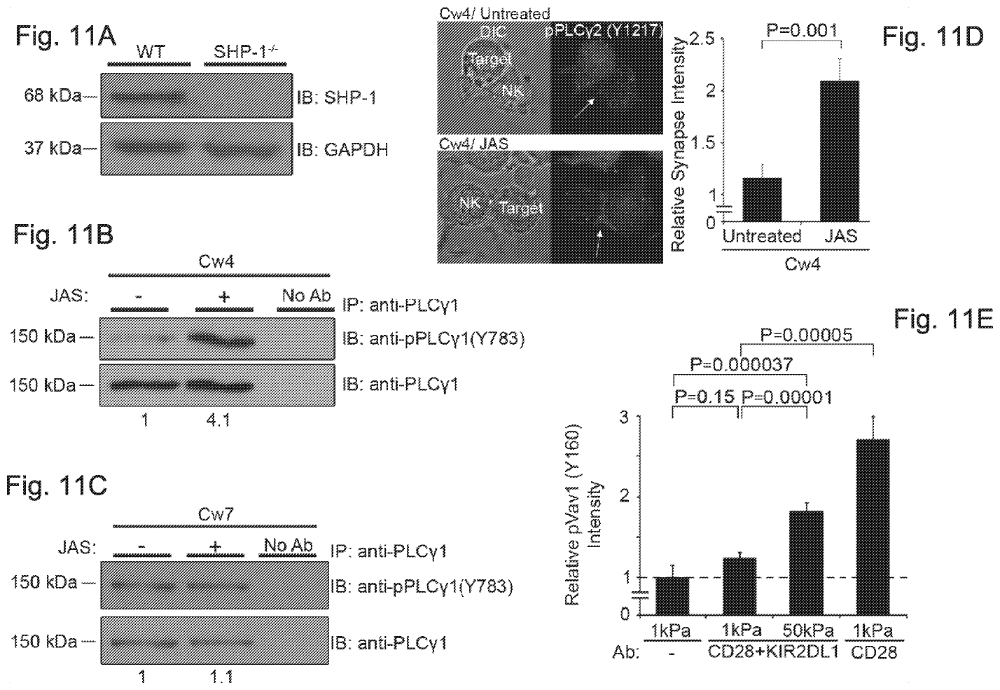

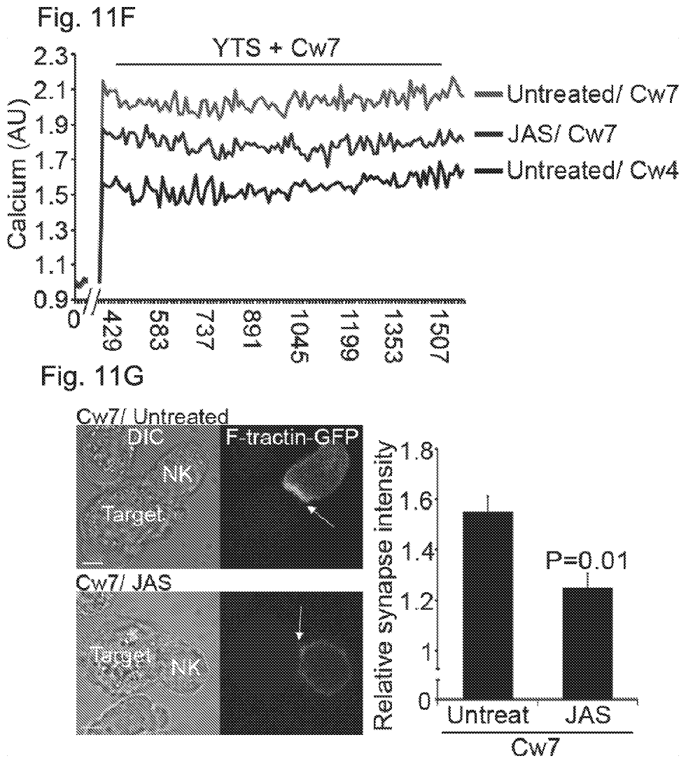

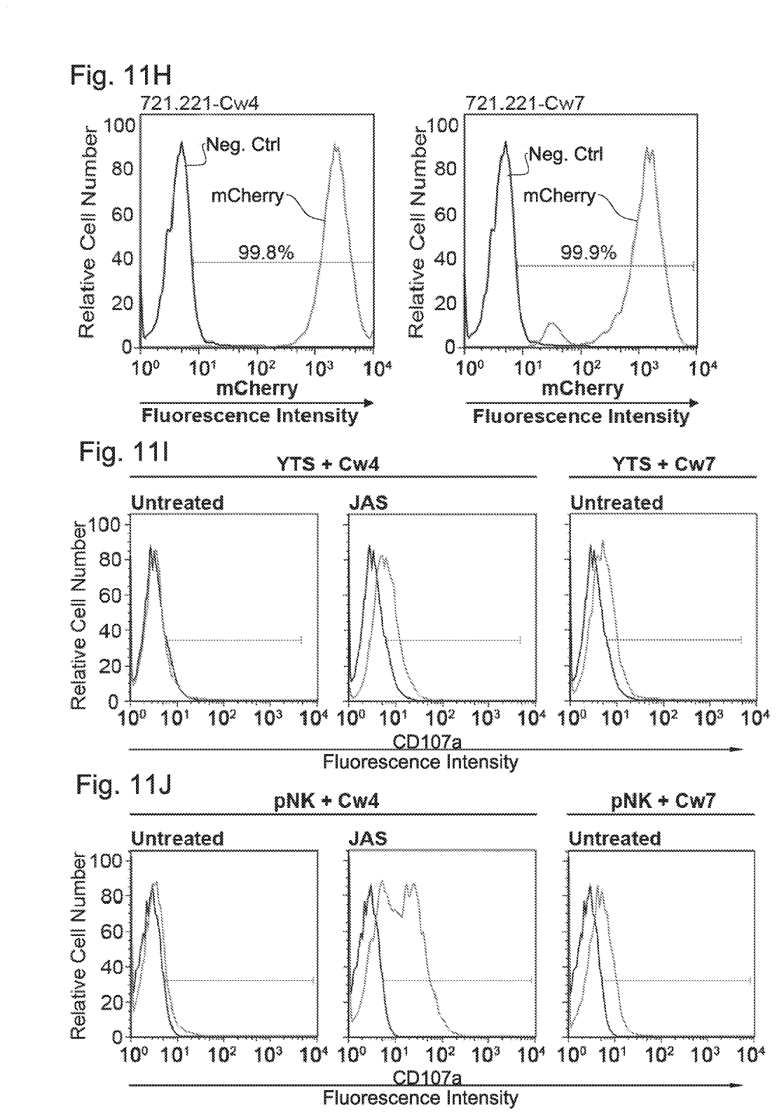

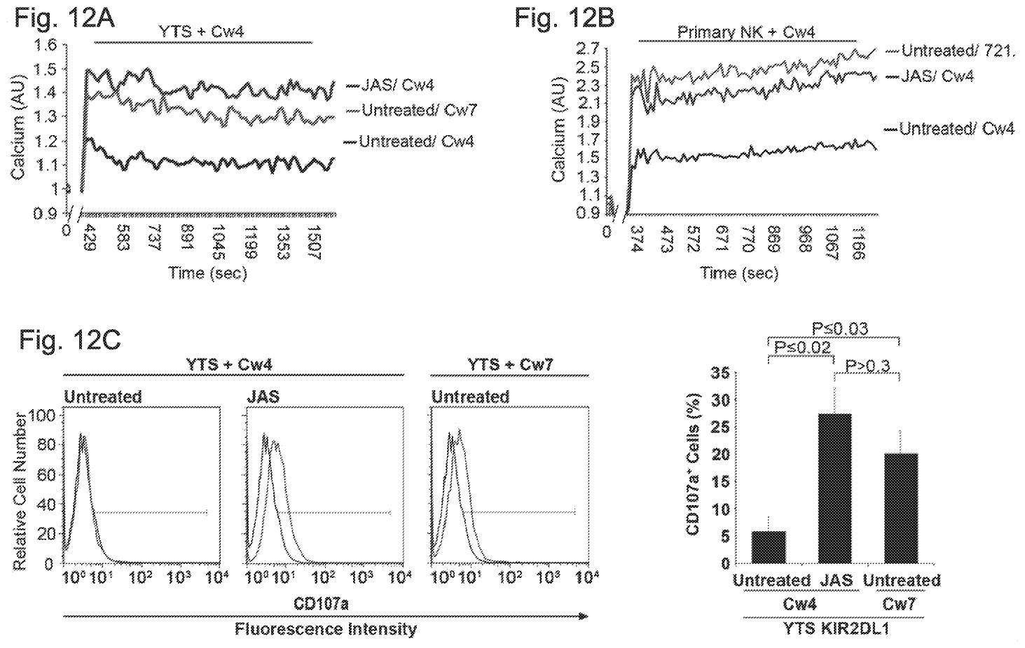

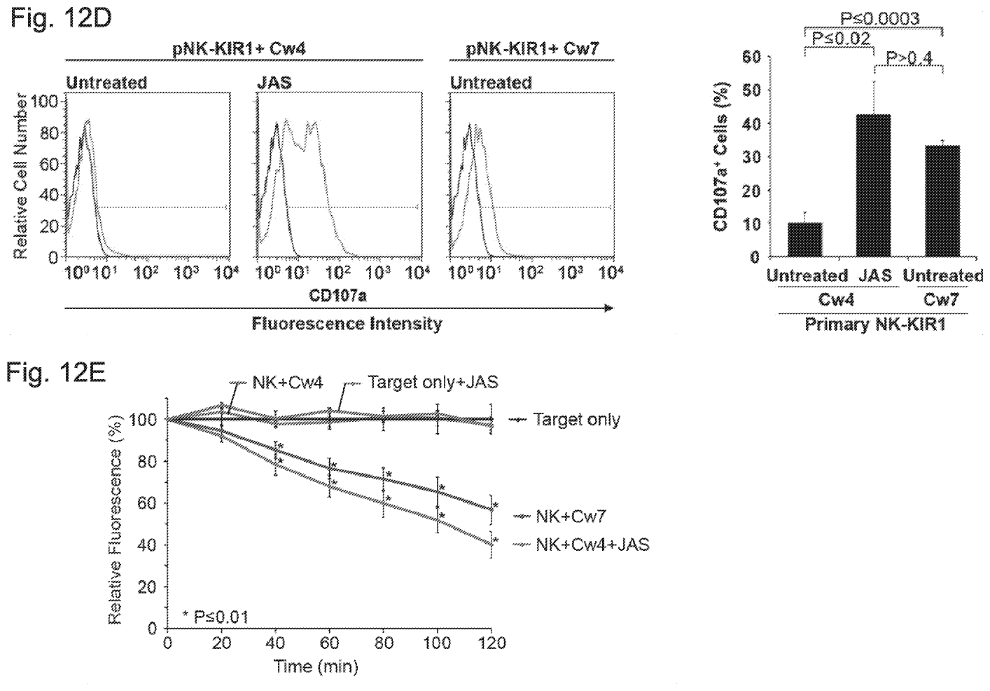

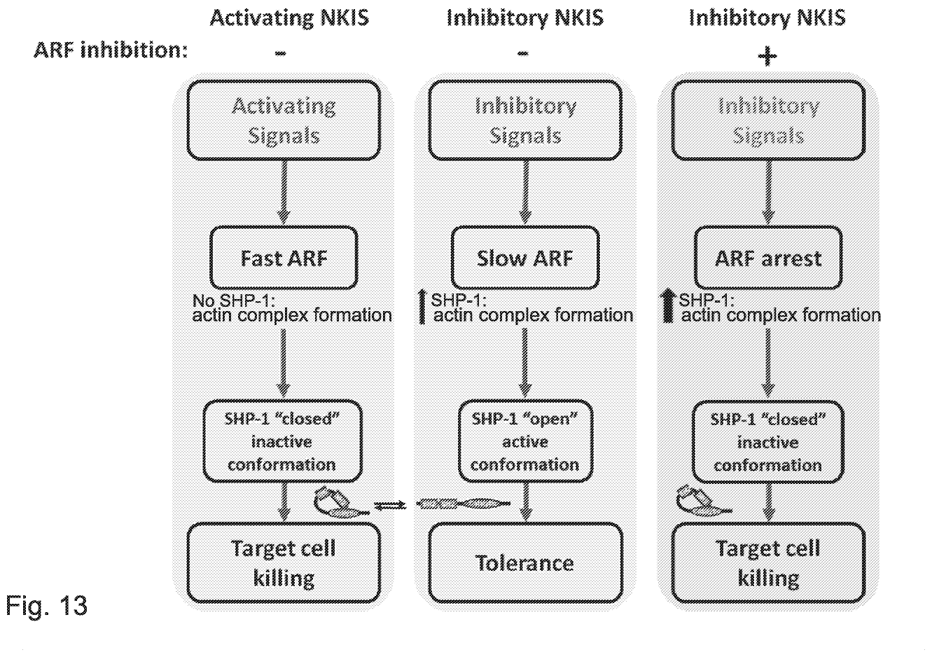

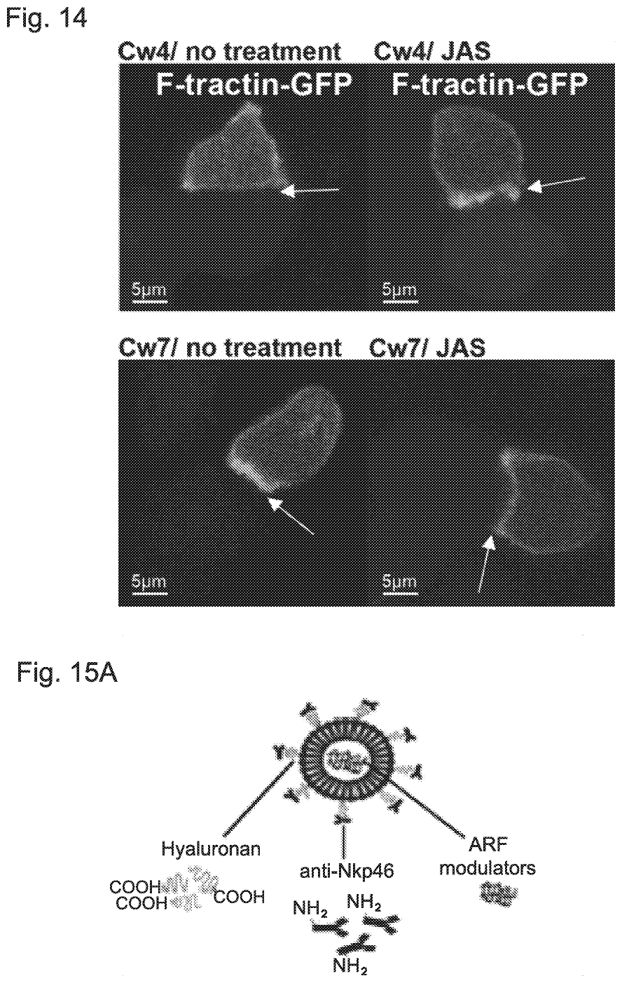

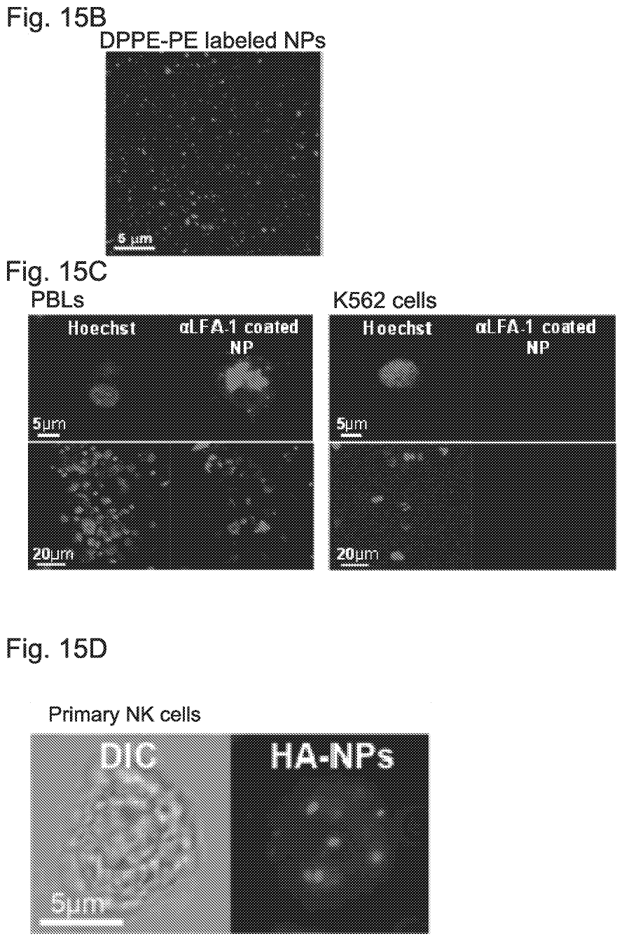

In FIG. 1A, YTS-2DL1 cells were stained with anti-KIR2DL1 antibody and a secondary Alexa-488 antibody. The expression level of the KIR2DL1 receptor was determined by FACS.

In FIG. 1B, YTS-2DL1 cells were incubated with 221-Cw4 target cells for 5 min at 37° C. The cells were lysed, and immunoprecipitates (IP) of SHP-1 were resolved by SDS-PAGE and stained with Coomassie blue. A band of ˜42 kDa was subjected to analysis by mass spectrometry, as described in the Experimental procedures section. Coverage of the identified β-actin protein following trypsin digestion is demonstrated. The figure shows the covered area as indicated in bold (70% coverage) on the beta-actin sequence as denoted by SEQ ID NO. 16.

In FIG. 1C, isolated primary KIR2DL1+NK cells were stained with anti-KIR2DL1 antibody, and a secondary Alexa-488 antibody. The expression level of the KIR2DL1 receptor was determined by FACS.

In FIG. 1D, primary NK-KIR2DL1 cells were incubated with 221-Cw4 or 721.221 (221) cells for 5 min at 37° C., and SHP-1 IP was subjected to IB using anti-β-actin or anti-SHP-1. As a negative control, IgG isotype antibody was used.

In FIG. 1E, YTS-2DL1 cells were incubated with 221-Cw4 or Cw7 cells for 5 min at 37° C. Cells were lysed and the SHP-1 IP was immunoblotted using anti-β-actin. SHP-1 was also immunoprecipitated from lysates of YTS-2DL1, 221-Cw4 or 221-Cw7 target cells alone as negative controls.

In FIG. 1F, YTS CFP-actin cells transiently expressing YFP-SHP-1 were incubated on slides pre-seeded with mCherry expressing 221-Cw4 (1F-1) or 221-Cw7 (1F-2) target cells for 5 min at 37° C. The cells were fixed, and FRET analysis was then performed. The graph (1F-3) summarizes the mean percentage FRET efficiencies ±SEM (n=43 cells for Cw4, n=37 cells for Cw7). Scale bars indicate 5 μm.

In FIG. 1G, MST analysis for the binding of YFP-SHP-1 and actin-derived peptides was performed as described in the Experimental procedures section. Lysates of 293T cells expressing YFP-SHP-1 were incubated with serially-diluted (400 μM-48 nM) peptides, including WT actin peptide (ITIM motif, KEKLCYVALDF as denoted by SEQ ID NO 1), mutant actin peptide (KEKLCFVALDF as denoted by SEQ ID NO 2), and irrelevant control peptide (EYQKASGVSG as denoted by SEQ ID NO 3). Binding curves were generated by the NanoTemper analysis software (MO.Affinity Analysis v2.1.3). The graph summarizes the changes of the fluorescent thermophoresis signals as a function of peptide concentration from at least three independent experiments. Data are means±SD.

FIGS. 2A-2C. SHP-1 binds β-actin in the inhibitory NKIS

FIGS. 2A-2B. show immunoprecipitation (IP) and immunoblotting (IB) analyses of the activated vs. inhibited YTS or primary NK cells, respectively. In FIG. 2A YTS-2DL1 cells were incubated with 721.221-Cw4 or Cw7 (221-Cw4 or Cw7) cells for 5 min at 37° C. Cell lysates were subjected to IP of SHP-1, and IB with anti-pTy (top panel) or anti-β-actin (bottom panel) antibodies. In FIG. 2B primary NK-KIR1 cells were incubated with 221-Cw4 or 721.221 cells as above, and subjected to IP of SHP-1 and IB with anti-β-actin or anti-SHP-1 antibodies.

FIG. 2C. shows YTS cells subjected to a pharmacological inhibitor of actin turnover—Jasplakinolide (JAS). YTS-2DL1 cells were incubated at 37° C. with 221-Cw4 or Cw7 cells for 5 min, 1 μM of JAS was added, and cells were incubated for 5 min. Cell lysates were subjected to IP of SHP-1 and IB with anti-β-actin or anti-SHP-1 antibodies. Shown data represent at least three independent experiments.

FIGS. 3A-3K. Differential F-actin and SHP-1 dynamics in the activating vs. inhibitory NKIS

FIGS. 3A-3B. show expression of the F-actin probe F-tractin GFP in YTS cells by Western blotting, and by FACS, respectively. In FIG. 3A, YTS-2DL1 cells were transfected with plasmids encoding F-tractin-GFP or GFP alone, and cell lysates were prepared after 24 hours. F-tractin GFP protein level and size were analyzed by IB with anti-GFP. In FIG. 3B, YTS-2DL1 cells were transfected with F-tractin-GFP, and cells stably expressing the protein were generated (YTS F-tractin GFP cells). F-tractin GFP expression level was determined by FACS.

FIG. 3C. shows imaging analysis of F-actin and myosin in the activating vs. inhibitory NKIS. YTS F-tractin GFP cells transiently expressing mCherry-Myosin IIA were seeded over coverslips pre-coated with activating anti-CD28 (top panel) or inhibitory anti-KIR1 antibody (bottom panel) and allowed to spread for 4 min at 37° C. before fixation. F-actin and myosin IIA distributions from multiple cells were profiled by ImageJ (see Experimental procedures). Figure shows the average normalized fold intensities of F-actin and myosin IIA along the diameter of the activating (top graph F-actin: n=68; myosin IIA n=66) vs. inhibitory NKIS (bottom graph F-actin: n=68; myosin IIA n=66). Data are means±SEM.

FIG. 3D. shows imaging analysis of F-actin distribution in the activating vs. inhibitory NKIS. YTS F-tractin GFP cells were incubated on slides with mCherry expressing 221-Cw7 or 221-Cw4 target cells for 5 min at 37° C., and fixed. Z stack images of NK-target conjugates and 3D projections of the NKIS planes were assembled. F-actin distribution from multiple cells were profiled by ImageJ as above. Figure shows the average normalized fold intensities of F-actin along the diameter of the activating vs. inhibitory NKIS (n=44 for Cw7, n=46 for Cw4). Data are means±SEM.

In FIG. 3E, primary NK cells were transfected with F-tractin GFP, and F-tractin GFP expression level was determined by FACS. In FIG. 3F, YTS F-tractin GFP cells were dropped over coverslips coated with either anti-CD28 or anti-KIR1, and live cell imaging was performed. Kymographs of F-actin dynamics were compiled along the contact site radius. Figure shows quantitative analysis of F-actin traces from kymographs obtained from lamelliopodia (LP) of activating vs. inhibitory contact sites as the time (sec) required for the average F-actin trace to traverse (μm) (anti-CD28: total traces=632 from 7 movies; anti-KIR1: total traces=296 from 7 movies). Data are means±SEM.

FIG. 3G. shown an analogous analysis of F-actin traces from LP of activating vs. inhibitory contact sites for primary NK F-tractin GFP cells over coverslips coated with anti-NKG2D or anti-NKG2A (anti-NKG2D: total traces=532 from 5 movies; anti-NKG2A: total traces=312 from 5 movies). Data are means±SEM.

FIG. 3H. shows an analysis of F-actin traces from the lamellum (LM) and cell body (CB) of activating vs. inhibitory contact sites for YTS F-tractin GFP cells over coverslips coated with anti-CD28 or anti-KIR1 (anti-CD28: total traces=231 from 7 movies; anti-KIR1 total traces=395 from =7 movies). Data are means±SEM.

FIG. 3I. shows an analysis of F-actin traces from LM and CB of activating vs. inhibitory contact sites for primary NK F-tractin GFP cells over coverslips coated with anti-NKG2D or anti-NKG2A (anti-NKG2D: total traces=157 from 5 movies; anti-NKG2A: total traces=98 from 5 movies). Data are means±SEM.

In FIG. 3J, YTS F-tractin GFP cells were dropped over isotype IgG coated coverslips, and imaged at a single focal plane at one frame per second. F-actin kymographs were compiled along the cell radius. Quantitative analysis of F-actin traces was performed and compiled into a graph to show the distribution of F-actin velocity (μm/sec) along the radius of the contact site (IgG: total traces=92 from 6 movies). Data are means±SEM.

FIG. 3K. shows quantitative analysis of SHP-1 traces for YTS-2DL1 cells expressing mCherry-SHP-1 over anti-CD28 or anti-KIR1 coated coverslips. SHP-1 kymographs were composited along the cell radius (anti-CD28: total traces=194 from 4 movies; anti-KIR1: total traces=121 from 5 movies). Data are means±SEM.

Data are representative of three independent experiments.

FIGS. 4A-4F. Inhibitory vs. activating NKISs are characterized by different F-actin dynamics

FIGS. 4A-4B. show imaging analysis of ARF in the activating vs. inhibitory primary NK and YTS cells. In FIG. 4A, YTS F-tractin GFP cells were dropped over coverslips coated with either anti-CD28 or anti-KIR1 and imaged at a single focal plane at one frame per second. Representative images are shown. Kymographs of F-actin dynamics were compiled along the contact site radius (represented as dashed lines). In FIG. 4B, primary NK cells expressing F-tractin GFP were dropped over anti-NKG2D or anti-NKG2A coated coverslips and analyzed as above.

FIGS. 4C-4D. show quantitative analysis of F-actin traces from kymographs of activating vs. inhibitory sites assembled into graphs representing F-actin velocity (μm/sec) along the radius of a contact site (YTS/anti-CD28: total traces=863 from 7 movies; YTS/anti-KIR1: total traces=691 from 7 movies; pNK/anti-NKG2D: total traces=748 from 5 movies; pNK/anti-NKG2A: total traces=537 from 5 movies; 0—cell center, 1—cell periphery).

FIGS. 4E-4F. show imaging analysis of SHP-1 retrograde flow in the activating vs. inhibitory YTS cells. In FIG. 4E, YTS-2DL1 cells expressing mCherry-SHP-1 were dropped over anti-CD28 or anti-KIR1 coated coverslips and imaged. SHP-1 kymographs were compiled along the cell radius. In FIG. 4F, quantitative analysis of SHP-1 traces was performed (anti-CD28: total traces=247 from 4 movies; anti-KIR1: total traces=315 from 5 movies). Data are representative of three independent experiments. Scale bars indicate 5 μm.

FIGS. 5A-5F. F-actin polymerization and myosinIIA contraction drive F-actin flow in NKIS

FIGS. 5A-5B. show analyses of ARF in the activating vs. inhibitory primary NK and YTS cells in the presence of JAS inhibitor of actin turnover. In FIG. 5A, YTS F-tractin GFP cells were dropped over coverslips coated with anti-CD28 or anti-KIR1 antibodies, and imaged at 1 frame/sec through a single focal plane. Following 50 sec of spreading, cells were treated with 1 μM of JAS. Representative images are shown. Kymographs of F-actin dynamics were compiled along the contact site radius. JAS treatment is indicated by an arrowhead. FIG. 5B. shows live cell imaging of primary NK cells expressing F-tractin GFP dropped over coverslips coated with anti-NKG2D or anti-NKG2A.

FIGS. 5C-5D. show quantitative analyses of the above experiments.

FIG. 5C. shows analyses of F-actin traces obtained from kymographs of YTS F-tractin GFP cells treated with JAS (anti-CD28: total traces=207 from 5 movies; anti-KIR1: total traces=519 from 6 movies). The y axis represents F-actin velocity (μm/sec), and the x axis—time from initial spreading (sec).

FIG. 5D. shows an analogous analysis of F-actin traces from kymographs of primary NK F-tractin GFP cells treated with JAS (anti-NKG2D: total traces=365 from 6 movies; anti-NKG2A: total traces=318 from 5 movies).

FIGS. 5E-5H. show analyses of NKIS area and ARF in the activating vs. inhibitory NKIS in the presence of Y-27632 (Y-27) inhibitor of myosin light chain (MLC) phosphorylation and/or JAS. In FIG. 5E is live cell imaging of YTS-2DL1 cells expressing F-tractin GFP treated with 25 μM Y-27 (anti-CD28: n=6 movies; anti-KIR1: n=6 movies). The graph of FIG. 5G, shows kymograph analysis. In FIG. 5F are YTS F-tractin GFP cells treated with 25 μM Y-27 or left untreated. Cells were seeded over coverslips pre-coated with anti-CD28 or anti-KIR1 and allowed to spread at 37° C. for 10 min before fixation, and 1 μM JAS was added 5 min after initial spreading. NKIS area was determined using ImageJ. The graph of FIG. 5, summarizes the average area (μm²) of activating vs. inhibitory NKIS (anti CD28: n=19 cells for untreated, n=20 cells for JAS, n=20 cells for Y-27; anti-KIR1: n=20 cells for untreated, n=20 cells for JAS, n=17 cells for Y-27). Scale bars are 5 μm. Data represent of at least three independent experiments.

FIG. 6. The effect of inhibition of F-actin polymerization on F-actin flow

YTS F-tractin GFP cells were dropped over coverslips coated with anti-CD28 or anti-KIR2DL1 antibodies, and imaged at 1 frame/sec through a single focal plane. Following cell spreading, the cells were treated with 0.5 μM of CytD. Kymographic analysis of F-actin traces at the LP was compiled into a graph to show F-actin velocity (μm/sec) before and after CytD treatment (anti-CD28: before CytD total traces=137, after CytD total traces=166 from 10 movies; anti-KIR2DL1: before CytD total traces=105 from, after CytD total traces=166 from 9 movies). Data are means±SEM.

FIG. 7. Inhibition of myosinIIA phosphorylation by Y-27

Figure shows the effects of Y-27 and JAS on MLC phosphorylation by IB analysis. YTS-2DL1 cells were treated with 25 μM of Y-27 for 15 min at 37° C., or left untreated. Cells were incubated at 37° C. with 22-Cw4 target cells for 5 min, 1 μM of JAS was added, and cells were incubated for 5 min. Cell lysates were analyzed by IB for phosphorylation of myosin light chain using anti-pMLC (Ser19) antibodies.

FIGS. 8A-8H. F-actin retrograde flow dictates SHP-1 catalytic activity and conformation

FIG. 8A. shows the effects of JAS and Y-27 on the SHP-1 catalytic activity. YTS-2DL1 cells were pretreated with 25 μM Y-27 or left untreated. Cells were incubated at 37° C. with target cells for 5 min, 1 μM of JAS was added, and samples were incubated for 5 minutes before lysis. IP of SHP-1 was performed, and precipitates were incubated with pNpp. SHP-1 activity was determined by measuring absorbance at 405 nm. The graph represents rates of SHP-1 catalytic activity from three independent experiments.

FIG. 8B. shows a schematic illustration of YFP-SHP1-CFP FRET sensor. Active “open” conformation of SHP-1 results in a large distance between the two fluorescent proteins with no FRET signal. The inactivated, “closed”, conformation of SHP-1 brings the N′- and C′-termini into proximity resulting in high FRET efficiency.

FIG. 8C. shows confocal images of activated vs. inhibited YTS cells subjected to JAS and Y-27 treatments, and FRET analysis of these cells. YTS-2DL1 cells transiently expressing YFP-SHP1-CFP were treated with 25 μM Y-27 or left untreated. Cells were incubated on slides with 221-Cw4 or Cw7 target cells expressing mCherry, following 5 min of conjugation at 37° C., cells were treated with 1 μM JAS, and incubated for 5 min before fixation. FRET analysis was performed (see Experimental procedures). The graph summarizes FRET efficiencies obtained from at least three independent experiments (n=33 for Cw4 untreated, n=21 for Cw7 untreated, n=25 for Cw4 JAS, n=36 for Cw4 Y-27, n=29 for Cw4 both). Scale bars indicate 5 μm.

In FIG. 8D, YTS-2DL1 cells were incubated at 37° C. with 221-Cw4 or Cw7 cells for 5 min, 0.5 μM of CytD was added, and the cells were incubated for an additional 5 min. The cells were lysed, and IPs of SHP-1 were subjected to IB using anti-β-actin or anti-SHP-1. Results shown are representative of three independent experiments.

In FIG. 8E, YTS-2DL1 cells were incubated at 37° C. with 221-Cw4 or Cw7 cells for 5 min, treated with 0.5 μM of CytD or left untreated, and SHP-1 activity was determined as detailed in the Materials and Methods. Graph summarizing percent of relative SHP-1 catalytic activity obtained by four independent experiments. Data are means±SEM.

In FIG. 8F, YTS F-tractin GFP cells were dropped over soft (1 kPa) or stiff (50 kPa) acrylamide gel surfaces coated with anti-CD28 or anti-KIR2DL1 antibodies, and imaged at 1 frame/sec through a single focal plane. Graph summarizes kymograph analysis of F-actin velocity (μm/sec) at the LP site (anti-CD28/1 kPa: total traces=295 from 10 movies; anti-CD28/50 kPa: total traces=108 from 9 movies; anti-KIR2DL1/1 kPa: total traces=168 from 12 movies; anti-KIR2DL1/50 kPa: total traces=59 from 8 movies). Data are means±SEM. In FIG. 8G, YTS YFP-SHP1-CFP cells were seeded over soft (1 kPa) or stiff (50 kPa) surfaces coated with anti-CD28 or anti-KIR2DL1 antibodies. Cells were allowed to spread for 10 min at 37° C. before fixation. Spread cells were imaged at a single confocal plane, FRET analysis was performed, and a graph summarizing the mean percentage FRET efficiencies ±SEM is presented (n=29 cells for KIR2DL1/1 kPa, n=25 cells for KIR2DL1/50 kPa, n=29 cells for CD28/1 kPa, n=30 cells for CD28/50 kPa) is shown in FIG. 8H. Scale bars indicate 5 μm. Data are representative of three independent experiments.

FIGS. 9A-9E. ARF regulates SHP-1 conformation specifically at the inhibitory NKIS but not at the activating NKIS

In FIG. 9A, YTS-2DL1 cells were incubated with 221-Cw4 inhibitory target cells for 10 min at 37° C. before lysis. Following SHP-1 IP, 1 μM of JAS was added directly to SHP-1 immunoprecipitates, incubated for 10 minutes, and SHP-1 activity was determined. Graph summarizing percent relative SHP-1 catalytic activity from three independent experiments. Data are means±SEM.

FIG. 9B. shows IB analysis of SHP-1, wherein the and C′-terminal ends were tagged with CFP/YFP (donor/acceptor pair). YTS-2DL1 cells were transiently transfected with YFP-SHP1-CFP (NLS mutated), and cell lysates were prepared after 24 hours; IB was performed with anti-GFP.

FIGS. 9C and 9E. show confocal images and FRET analysis of NK cells expressing YFP-SHP1-CFP after activating vs. inhibitory stimulus, and JAS treatment. In FIG. 9C, YTS YFP-SHP1-CFP cells were incubated on slides pre-seeded with 221-Cw7 target cells expressing mCherry. Following 5 min conjugation at 37° C., the cells were treated with 1 μM JAS or left untreated and incubated for an additional 5 min before fixation; FRET analysis was then performed. Graph summarizing the mean percentage FRET efficiency ±SEM from three independent experiments is presented (n=21 cells for Cw7 untreated, n=36 cells for Cw7 JAS). Scale bars indicate 5 μm.

In FIG. 9D, YTS YFP-SHP1-CFP cells were incubated on slides with 221-Cw4 or Cw7 target cells expressing mCherry, and treated with JAS as in FIG. 8C. FRET analysis was performed as described in Experimental procedures. To determine the distribution of FRET signal at the NKIS vs. non-NKIS areas, the relative FRET signal at the synapse was determined by measuring the ratio between the averaged FRET efficiency at the NKIS relative to a site on the cell that did not include the NKIS, using ImageJ. Data are means±SEM.

In FIG. 9E, YTS YFP-SHP1-CFP cells were seeded over coverslips pre-coated with anti-CD28 or anti-KIR2DL1 antibodies. As a control, unstimulated cells were seeded over uncoated slides. Cells were allowed to spread for 5 min at 37° C., treated or not with 1 μM JAS, and incubated for an additional 5 min before fixation. FRET analysis was performed, and a graph summarizing the mean FRET efficiencies ±SEM is presented (n=51 cells for KIR2DL1/untreated, n=64 cells for KIR2DL1/JAS, n=71 cells for CD28/untreated, n=69 cells for unstimulated/untreated). Scale bars indicate 5 μm. Data are representative of three independent experiments.

FIGS. 10A-10F. Inhibition of F-actin dynamics in the inhibitory NKIS results in enhanced phosphorylation of VAV1 and PLCγ1/2

FIGS. 10A and 10C. show images and quantitative analysis of phosphorylation of VAV1, SHP-1 substrate, in NK cells subjected to activating vs. inhibitory stimulus, and JAS treatment.

In FIG. 10A, YTS-2DL1 WT or SHP-1 knockout (KO) cells were incubated on slides with mCherry expressing 221-Cw4 or 221-Cw7 target cells at 37° C. After 5 min incubation, the cells were treated with 1 μM of JAS (bottom panels), or left untreated (top panels), and incubated for an additional 5 minutes before fixation. The cells were stained with anti-pVAV1 (Y160) and secondary Alexa-488 antibody, and accumulation of phosphorylated VAV1 at the NKIS was determined. NK cells were distinguished from targets based on mCherry expression by the target cells. Scale bars indicate 5 μm.

In FIG. 10B, the graph summarizes the relative pVAV1 (Y160) synapse fluorescence intensities (YTS WT/Cw4 untreated: n=25, WT/Cw4 JAS: n=27, WT/Cw7 untreated: n=28, WT/Cw7 JAS: n=27, SHP-1 KO/Cw4 untreated: n=27, SHP-1 KO/Cw4 JAS: n=26). Data are means±SEM.

In FIG. 10C, primary NK-2DL1 cells were incubated on slides with 221-Cw4 or 721.221 target cells at 37° C. After 5 minutes, the cells were treated with 1 μM JAS and incubated for an additional 5 minutes before fixation. Accumulation of phosphorylated VAV1 (Y160) at the NKIS was determined as in FIGS. 10A and 10B. The graph summarizes the relative pVAV1 (Y160) synapse fluorescence intensities (Cw4 untreated: n=18; Cw4 JAS: n=18; 721.221 untreated: n=24). NK cells were distinguished from targets based on the DIC channel. Scale bar indicates 5 μm. Data are means±SEM.

FIGS. 10D-10E. show IP and IB of phosphorylation of VAV1 and PLCγ1/2, both SHP-1 substrates, in NK cells subjected to activating vs. inhibitory stimulus, and JAS treatment.

In FIG. 10D, YTS-2DL1 cells were incubated with 221-Cw4 target cells at 37° C. for 5 min; 1 μM of JAS was added, and the cells were incubated for an additional 5 min before lysis. IP of pTy immunoblotted using anti-VAV1. As a loading control, whole cell lysates (WCL) were analyzed by western blotting with anti-GAPDH antibody.

In FIG. 10E, YTS-2DL1 cells were incubated with 221-Cw4 or 221-Cw7 target cells at 37° C. for 5 min, and treated with 1 μM of JAS as in FIG. 10D. IP of pTy immunoblotted using anti-PLCγ1. As a loading control, WCL were analyzed by western blotting with anti-GAPDH antibody.

In FIG. 10F YTS-2DL1 cells were incubated with 221-Cw4 or 221-Cw7 target cells at 37° C. for 5 min, and treated with 1 μM of JAS as in FIG. 10D. IP of PLCγ2 immunoblotted using anti-pPLCγ2 (Y1217). Data are representative of at least three independent experiments.

FIGS. 11A-11J. Actin dynamics regulates phosphorylation levels of PLCγ1/2 in the inhibitory NKIS

In FIG. 11A YTS-2DL1 WT or SHP-1 knockout (SHP-1^{-/-}) cells, produced by CRISPR/Cas9 system, were analyzed by western blotting with anti-SHP-1 and anti-GAPDH antibodies.

FIG. 11B-11C. show YTS-2DL1 cells were incubated at 37° C. with 221-Cw4 (FIG. 11B) or 221-Cw7 (FIG. 11C), target cells for 5 min, 1 μM of JAS was added, and the cells were incubated for an additional 5 min. The cells were lysed, and IP of PLCγ1 was then immunoblotted with anti-pPLCγ1 (Y783).

FIG. 11D. show YTS-2DL1 cells were incubated on slides pre-seeded with mCherry-expressing 221-Cw4 target cells, and allowed to form conjugates at 37° C. Following 5 min incubation, the cells were treated with 1 μM of JAS and incubated for an additional 5 min before fixation. The cells were stained with anti-pPLCγ2 (Y1217) and a secondary Alexa-488 antibody, and the accumulation of phosphorylated PLCγ2 at the NKIS was determined. NK cells were distinguished from targets based on mCherry expression in the target cells. Graph summarizes the relative synapse intensities (n=11 for Cw4 untreated, n=14 for Cw4 JAS). Scale bar represents 5 μm. Data are means±SEM.

In FIG. 11E, YTS-2DL1 cells were seeded over soft (1 kPa) or stiff (50 kPa) surfaces coated with both anti-CD28 and KIR2DL1 antibodies, or with anti-CD28 antibody alone. Uncoated surfaces were used as a control. Cells were allowed to spread for 10 min at 37° C. before fixation. The cells were stained with anti-pVAV1 (Y160) and secondary Alexa-488 antibody. Spread cells were imaged at a single confocal plane at the NK contact site with the hydrogel surfaces, and fluorescence intensity was measured using ImageJ. Graph summarizing the relative pVAV1 (Y160) fluorescence intensities at the contacts sites (n=22 cells for uncoated/1 kPa; n=24 cells for CD28+KIR2DL1/1 kPa; n=32 cells for CD28+KIR2DL1/50 kPa; n=20 cells for CD28/1 kPa). Results are presented relative to fluorescence intensity measured in cells spread over uncoated surfaces; dashed line represents the basal phosphorylation of VAV1 as detected in these cells. Data are means±SEM.

In FIG. 11F, YTS-2DL1 cells were loaded with the calcium-sensitive dye, Fluo-3-AM, and analyzed for basal intracellular calcium levels for 1 min. The NK cells were then mixed with 221-Cw4 or 221-Cw7 target cells and incubated at 37° C. Following 5 min incubation, indicated cells were treated with 1 μM JAS, or left untreated, and the calcium levels were further analyzed by spectrofluorometry. A representative experiment out of three independent experiments is shown.

In FIG. 11G YTS F-tractin GFP cells were incubated on slides with mCherry-expressing 221-Cw7 target cells at 37° C. After 5 min incubation, the cells were treated with 1 μM of JAS, or left untreated, and incubated for an additional 5 minutes before fixation. Accumulation of F-actin at the NKIS was determined. NK cells were distinguished from targets based on mCherry expression by the target cells. Graph summarizes the relative F-tractin GFP synapse intensities (Cw7 untreated: n=19, Cw7 JAS: n=20). Scale bar indicates 5 μm. Data are means±SEM.

In FIG. 11H, 221-Cw4 and Cw7 cells were transfected with plasmid encoding mCherry, and cells stably expressing high levels of the protein were generated. mCherry expression level was determined by FACS.

In FIG. 11I, YTS-2DL1 cells were incubated with mCherry-expressing 221-Cw4 or Cw7 target cells for total of 2 hrs; JAS was added 5 min after incubation was initiated. Degranulation was determined by measuring the percentage of CD107a positive NK cells by FACS. The NK cells were distinguished from the target cells according to mCherry expression. A representative result out of four independent experiments is shown.

In FIG. 11J, Primary NK-2DL1 cells were incubated with mCherry-expressing 221-Cw4 or Cw7 target cells for total of 2 hrs; JAS was added 5 min after incubation was initiated. The percentage of CD107a positive NK cells was determined by FACS. The NK cells were distinguished from the target cells based on mCherry expression. A representative result out of six independent experiments is shown.

FIGS. 12A-12E. F-actin flow dictates NK cell activation and function

FIGS. 12A-12B. show analyses of Ca²⁺ flux in YTS and NK primary cells subjected to inhibitory stimulus and JAS. In FIG. 12A, YTS-2DL1 cells were loaded with calcium-sensitive Fluo-3-AM and analyzed for basal intracellular calcium levels for 1 min. Cells were mixed with 221-Cw4 or 221-Cw7 target cells and incubated at 37° C. for 5 min Cells were treated with 1 μM JAS, and calcium levels were analyzed by spectrofluorometry. Data represent at least three independent experiments is shown. In FIG. 12B, primary NK-2DL1 cells were loaded with Fluo-3-AM and analyzed for intracellular calcium levels, using 221-Cw4 or 721.221 target cells.

FIGS. 12C-12D show the effect of JAS on secretion of cytolytic granules in the same cells. In FIG. 12C YTS-2DL1 cells were incubated with mCherry-expressing 221-Cw4 or Cw7 target cells for 2 hrs, JAS was added following 5 min from incubation-start. Degranulation was determined by measuring the percentage of CD107a positive NK cells by FACS (left). The NK cells were distinguished from the target cells by mCherry expression. The graph (right) summarizes the percent CD107a positive cells from four independent experiments.

FIG. 12D. shows an analogous experiment for the primary NK-2DL1 cells. The graph (right) summarizes the percent CD107a positive cells from six independent experiments.

In FIG. 12E, YTS-2DL1 cells were incubated on slides with 221-Cw4 or Cw7 target cells that were stained with the vital dye calcein-AM. Following five minutes of conjugation at 370 C, indicated cells were treated with 1 μM JAS, and imaged every 2 minutes for a total of 120 min Target cells incubated on slides alone, either treated or untreated with 1 μM JAS, served as negative controls. The graph summarizes the fluorescence intensity of target cells after 120 min, relative to the fluorescence intensity of the cells at time=0 min (n=7 movies for target only, n=8 movies for target only+JAS, n=5 movies for NK+Cw4, n=11 movies for NK+Cw7, and n=14 movies for NK+Cw4+JAS). The results are presented relative to the florescence measured in the ‘Target cell only’ sample to determine the specific loss of fluorescence resulting from NK cell mediated cytotoxicity. * Represents statistical significance relative to ‘NK+Cw4’ sample. Data are means±SEM.

FIG. 13. Suggested mechanisms for ARF mediated-regulation of SHP-1 conformation and activity tuning NK cell cytotoxicity

A schematic illustration of the inventive concept conceived on the basis of the above experimental results.

Activating NKIS: Activating signals, initiated by NK cell activating receptors, result in a fast actin flow that prevents SHP-1 binding to the actin network. Under these conditions, SHP-1 remains in the closed inactive conformation, enabling NK cell activation and target cell killing Inhibitory NKIS: Following inhibitory receptor engagement, inhibitory signals result in slow actin flow, enabling SHP-1:13-actin complex formation. Forces exerted by actin movement switch SHP-1 from the closed inactive conformation into the open active one. As a consequence, SHP-1 dephosphorylates key signaling molecules, thereby inhibiting NK cell activation. ARF inhibition at the inhibitory NKIS increases SHP-1 binding to actin; however, due to the lack of ARF-generated forces, SHP-1 is maintained in the closed inactive state. Since changes in actin flow dynamics are rapid events, this may allow for fast and flexible transition from an inhibitory to activating NK cell response.

FIG. 14. ARF inhibition results in F-actin accumulation at the inhibitory NKIS

Figure shows images of double labeled NK-target conjugates. NK cells expressing F-tractin-GFP were incubated with mCherry expressing 721.221-Cw4 inhibitory or -Cw7 activating target cells in the presence or absence of JAS. F-tractin-GFP accumulation was determined.

FIGS. 15A-15D. Lymphocyte targeting by liposomal nanoparticles

FIGS. 15A-15B. show preparation and detection of liposomal NPs.

FIG. 15A. is a schematic representation of NPs targeting NK cells. FIG. 15B shows NP detection by confocal microscopy using incorporation of DPPE labeled with Rhodamine red (DPPE-PE) into the lipid mixture.

FIG. 15C. shows selective uptake of anti-LFA1 coated NPs by PBLs expressing LFA-1 (upper image represents a single cell, lower-whole field,) but not by K562 cells (deficient in LFA-1). Cell nucleus was labeled with Hoechst to enable cell detection.

FIG. 15D. shows images of successful HA-NPs uptake by primary NK cells.

### DETAILED DESCRIPTION OF THE INVENTION

The present invention stems from unprecedented findings of a novel mechanism to control activation and function of NK cells, and the inhibitory NKIS in particular. In a series of experiments the inventors revealed a new player in the signal transduction in NK cells, namely a dynamic actin flow or actomyosin retrograde flow (ARF), which has been demonstrated to regulate a number of functional features of NK signal transduction, most prominently the activity of the SHP-1 tyrosine phosphatase, a key player of NK cell inhibition. So far, actomyosin network dynamics has been never suggested or described as an active or functional component that integrates and dictates inhibitory vs. activating signals in NKIS.

The realization that actomyosin network dynamics plays an important role in shaping activation and functional properties of NK cells has been substantiated on several levels in presently described EXAMPLES 1-7 and FIGS. 1-12 using multidisciplinary approaches, including cutting-edge imaging technologies such as intramolecular SHP-1 fluorescence resonance energy transfer (FRET) sensor, traditional biochemical analysis together with NK functional analyses as well as Mass spectrometry. Specifically, (1) the inventors monitored the actomyosin network dynamics in inhibitory vs. activating NKIS in the presence of specific pharmacological inhibitors, such as JAS, Y-27 and Cytochalasin D (CytD); (2) the inventors demonstrated that ARF impacts on NK signal transduction by governing SHP-1 conformational structure and activity; (3) the inventors further demonstrated that modulation of ARF, and in particular inhibition of ARF in the inhibitory NKIS, has significant impact on NK cell functional phenotype, such as cytotoxicity; (4) ultimately, the inventors suggested an inventive concept explaining how actomyosin dynamics via molecular mechanisms involving SHP-1 dictate the formation of either inhibitory or activating NKIS and further how these molecular mechanisms are translated into a defined biological outcome in NK cells (FIG. 13).

More specifically, the inventors revealed that actin network serves as a functional unit mediating inhibitory signals, thereby balancing between sustained signaling and termination. The inventors have now identified a novel molecular interaction between β-actin and SHP-1 at the inhibitory NKIS. Slow ARF was observed during the NK inhibitory vs. activating response, raising the possibility that ARF regulates SHP-1 activity. Indeed, the inventors now found that using the modulators of the invention, ARF inhibition following NK cell inhibition leads to a remarkable elevation in SHP-1: β-actin complex formation, resulting in transformation of SHP-1 conformation into a ‘closed’—inactive state, which is consistent with a significantly reduced SHP-1 enzymatic activity in the inhibitory NKIS. It has been known that the engagement of inhibitory receptors by MHC class I molecules in NK cells results in SHP-1 mediated dephosphorylation of VAV1, and prevents the release of calcium from the endoplasmic reticulum (ER) stores [5-7]. The inventors found that ARF inhibition induces phosphorylation of VAV1 and PLCγ1/2 at the inhibitory NKIS, suggesting that ARF regulation of SHP-1 activity controls the activation of key signaling events in NK cells. Further, blocking of actin centripetal flow resulted in elevated intracellular calcium flux during the NK inhibitory response, which is consistent with enhanced PLCγ1/2 phosphorylation. Furthermore, blocking of ARF increased the secretion of lytic granules by NK cells toward inhibitory target cells, indicating that ARF plays a key role in the conversion of inhibitory into activating NKIS. Altogether, these data point to F-actin dynamics, and specifically ARF, at the NKIS site as a determinant of NK inhibitory vs. activating responses by controlling the conformational state and activation status of a key signaling molecule, SHP-1, thereby modulating NK cell cytotoxicity.

Further, the inventors found that following NK cell activation, as opposed to NK cell inhibition, F-actin network exhibits a faster retrograde flow (ARF) in LP, and that F-actin decelerates with movement towards the NKIS center. These findings obtained in a JAS-free system, indicate that blockage of ARF represents a physiological condition that controls NK cell signaling. Further, imaging data demonstrated that ARF inhibition by JAS induces VAV1 and PLCγ hyper-phosphorylation specifically at the NK: target contact site (NKIS), thus supporting the physiological relevance of JAS in dictating NK cell signaling and function.

The inventors further demonstrated how ARF controls SHP-1 activity. In naïve NK cells SHP-1 is located in the cytoplasm in a folded auto-inhibited conformation. Recruitment of SHP-1 to the inhibitory NKIS releases its catalytic domain, thus enabling its phosphatase activity, and leading to dephosphorylation of its substrates. The SHP-1 closed conformation is mediated by association of its N terminal SH2 domain with its catalytic domain, thereby preventing SHP-1 binding to its substrates. The pulling forces of actin exerting tension on SHP-1 could potentially prevent the intramolecular interaction between the SH2 domain and the catalytic domain, thereby leading to SHP-1 ‘open’—active conformation. Under ARF inhibition, however, no forces are applied to SHP-1, thus permitting intra-molecular interaction between SH2 and the catalytic domain and maintaining SHP-1 in a ‘closed’-inactive conformation. The fact that no effect of JAS was detected in the activating NKIS further suggests that in absence of inhibitory signals, SHP-1 spontaneously acquires a ‘closed’—inactive conformation.

In other words, the presently disclosed data support a mechano-dependent mechanism of NK cell signaling, which is driven by actin network dynamics, and wherein SHP-1 serves as a mechano-sensor. The binding of F-actin to SHP-1 may be potentially regulated by the velocity of actin flow. Specifically, intensive actin dynamics in the activating NKIS may result in disassociation between SHP-1 and β-actin, while in the inhibitory NKIS, weak actin dynamics enable the formation of SHP-1: β-actin molecular complex. More specifically, actin regulation of SHP-1 is dependent on two main effects: (1) slow actin flow to enable formation of the SHP-1: β-actin complex, and (2) physical forces of ARF that detach the intramolecular interaction between SHP-1 SH2 domain from its catalytic domain.

The inventors presently propose a negative feedback mechanism that enhances the ability of NK cells to rapidly respond to changes in their proximal environment, i.e. simultaneous exposure to multiple target cells, activating or inhibitory (FIG. 13). More specifically, the inventors suggest that the inhibitory synapse is characterized in that ARF is in a state of equilibrium between slow movement and arrest, whereby slow moving actin forms an intensive interaction with the SHP-1 molecule, thus switching its conformation to an ‘open’-active form and blocking NK activation. When NK cells are activated, ARF is increased, leading to disassociation between SHP-1 and actin, which in turn increases SHP-1 accessibility to its substrates by potential incoming inhibitory signals. While NK synapse formation is a relatively slow process, changes in actin flow dynamics and subsequent SHP-1 conformation are rapid events, allowing fast on/off switch of inhibitory signaling. This actin mechano-transduction mechanism may enhance the ability of NK cells to respond to local changes in its proximal environment when shifting to a new target. Further, this suggested mechanism could be highly relevant when NK cells encounter multiple target cells simultaneously, or have sequential engagement with target cells, which occur with cancerous and healthy cells coexisting in the same environment. It is further conceived that rapid activation and/or deactivation of other signaling molecules/enzymes via changes in actin flow could be a common mechanism for dynamic regulation of leukocyte function.

Essentially, the present invention provides means for controlling activation of a host cellular immunity that can be implemented in various conditions, in health and disease, predominantly in host organisms possessing a vertebrate immunity system. Thus, in a first aspect, the present invention relates to a modulator that modulates at least one of actin and myosin retrograde flow (ARF) in a cell, for use in a method for modulating hematopoietic cell activation. In other words, the invention provides a modulator for use in a method for modulating hematopoietic cell activation. In some specific and non-limiting embodiments, such hematopoietic cell may be a lymphocyte cell forming an activating or inhibitory immunological synapse (IS). Thus, the invention encompasses ARF modulators for use in modulating lymphocyte activation. Such modulator is characterized in that it modulates at least one of ARF in a lymphocyte cell forming an activating or inhibitory IS. The term ‘modulator’ is meant herein to convey an agent that is capable of exerting a modifying or controlling influence on actin and myosin retrograde flow (ARF), and uses thereof as a modulator for lymphocyte cell activation, being it an inhibitory or activating with respect to the lymphocyte cell activation. Therefore, a modulator in accordance with the invention is used herein to either inhibits, diminishes, decreases, eliminates, disturbs, attenuates or alternatively, induce, elevates, activate, enhance, enlarge, increase lymphocyte activation. The key feature of the presently conceived modulators is revealed in their capability to modulate cellular actin and/or myosin network dynamics, or cellular actin and/or myosin retrograde flow (ARF). According to the present inventive concept, such type of modulators is particularly effective for use in altering or controlling activation of a lymphocyte making part of an immunological synapse or an immune synapse (IS), being it an activating or inhibitory IS.

The term ‘immunological synapse’ refers to an interface between an antigen-presenting cell (APC), a target cell, or both and a lymphocyte such as an effector T cell, a Natural Killer (NK) cell or even a B cell. It is thus meant that using the modulators according to the above can shift the balance between activating and inhibitory stimuli to which said lymphocyte is subjected at IS.

In other words, the activity of the presently conceived modulators encompasses situations, wherein the actin and/or myosin ARF modulators used herein, act as activators of a lymphocyte subjected to a sum total of inhibitory stimuli at IS, and also when the modulators used act as inhibitors of a lymphocyte subjected to a sum total of activating stimuli at IS. The unifying feature of all potential activities exerted by such modulators used herein is that their activating or inhibiting effects on lymphocyte cell activation are achieved via modulation of cellular actin and/or myosin network dynamics, or ARF. It is contemplated that the effect of the use of this type of modulators may be revealed in measurable changes in cytotoxic activity or in intracellular indices thereof in treated lymphocytes forming IS compared to untreated lymphocytes in the same condition (see EXAMPLES 6-7). For actin and/or myosin ARF modulators that uses thereof results in activation of lymphocytes, and as such, can be used as activating modulators, for example, these changes may be estimated as at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900% or about 1000% increase in activation of the lymphocytes forming IS. For the actin and/or myosin ARF modulators that uses thereof results in inhibition of lymphocytes, and as such, can be used as inhibiting modulators, these changes may be as at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% decrease in activation of the lymphocytes forming IS.

These changes may be further expressed in terms of activation of a lymphocyte subjected to an inhibitory IS, or alternatively inhibition of a lymphocyte subjected to an activating IS.

As indicated above, a key feature of the modulators of the invention (either inhibitors or inducers) is the ability to modulate ARF or the cellular actin and/or myosin network dynamics.

Further, the terms ‘cellular actin and/or myosin network dynamics’, or ‘actomyosin network dynamics’ or ‘actin and/or myosin retrograde flow’ (ARF), which are interchangeably used though the specification, all denote spatiotemporal changes in the actin and/or myosin cytoskeleton structure, distribution and contractility (also actomyosin contractility). These terms as known in the art are meant to convey the concept of a highly dynamic protein polymer network constructing the actin and/or myosin cytoskeleton and its numerous regulatory binding proteins. The actin and/or myosin network dynamics may be further described in terms of a collective dynamics of active cytoskeletal networks, which is an intricate interplay between cytoskeletal actin filaments and crosslinking proteins required for their mechanical stability, and further molecular motor proteins that introduce an active component. One example of a molecular motor protein is the skeletal muscle myosin, or myosin II. Myosins and other molecular motor proteins bind to a polymerized cytoskeletal filament and use the energy derived from repeated cycles of ATP hydrolysis to produce steady movement of cytoskeletal filaments against each other. This results in a highly flexible and adaptable scaffold that undergoes constant remodeling, and is capable of generating a force that underlies such phenomena as muscle contraction, ciliary beating, and cell division. According to the presently introduced inventive concept, activation of a lymphocyte cell, especially the one in a configuration of IS, is further contingent on this collective dynamics of active cytoskeletal networks.

More specifically, in some embodiments, the presently contemplated modulators used by the invention are acting on lymphocyte cell activation by enhancing or inhibiting actin and/or myosin flow, or analogously inducing a faster or slower actin and/or myosin flow or ARF in lymphocytes forming IS compared to untreated lymphocytes in the same condition. These effects can be measured, for example, using actin and/or myosin labeling and various imaging methods in situ (see EXAMPLE 3), and are more pronounced in specific cell compartments such as lamelliopodia (LP). According to the presently introduced inventive concept, in some embodiments, the ARF modulators used by the invention may lead to activation of lymphocytes, and as such, may be used as activating modulators of lymphocyte activation. In some specific embodiments, such modulators may be ARF inhibitor/s. More specifically, these modulators are decreasing, reducing or inhibiting at least one of actin and myosin flow or ARF to the extent of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% in the treated versus untreated lymphocytes forming IS, particularly in LP. In yet some further specific embodiments, ARF modulators that can be used as inhibiting modulators of lymphocyte activation may be ARF inducers that may increase or enhance actin and/or myosin flow or ARF—at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900% or about 1000%, particularly in LP of the treated versus untreated lymphocytes forming IS.

These effects may be further expressed in terms of inducing a faster ARF particularly in LP of a lymphocytes subjected to an inhibitory IS, or alternatively a slower ARF particularly in LP of a lymphocyte subjected to an activating IS.

As indicated above, the invention provides ARF modulators for use in modulating hematopoietic cell activation. “Hematopoietic cells” are cellular blood components all derived from hematopoietic stem cells in the bone marrow. It should be appreciated that in certain embodiments, hematopoietic cells as used herein include cells of the myeloid and the lymphoid lineages of blood cells. More specifically, myeloid cells include monocytes, (macrophages and dendritic cells (DCs)), granulocytes (neutrophils), basophils, eosinophils, erythrocytes, and megakaryocytes or platelets. The Lymphoid cells include T cells, B cells, and natural killer (NK) cells. Thus, in certain embodiments, the cells treated by the modulators of the invention may be any hematopoietic cell described herein. Generally, blood cells are divided into three lineages: red blood cells (erythroid cells) which are the oxygen carrying, white blood cells (leukocytes, that are further subdivided into granulocytes, monocytes and lymphocytes) and platelets (thrombocytes).

In certain embodiments, the hematopoietic cells treated by the modulators of the invention may be non-erythroid hematopoietic cells. The term “non-erythroid hematopoietic cell” refers to the cells derived from white blood cell precursors and from megakaryocytes and include at least one of granulocytes (neutrophils, basophils, eosinophils), monocytes, lymphocytes, macrophages, dendritic cells and platelets.

Still further, in some particular embodiments, the invention provides ARF modulators for use in modulating lymphocyte cell activation.

The nature of these activating or inhibiting effects of the ARF modulators used by the invention on lymphocyte cell activation is further dependent on the type of a lymphocyte, being it a lymphocyte of the innate or adaptive immune systems, or immunity.

“Lymphocytes” as used herein, are mononuclear nonphagocytic leukocytes found in the blood, lymph, and lymphoid tissues. They comprise the body's immunologically competent cells and their precursors. They are divided on the basis of ontogeny and function into two classes, B and T lymphocytes, responsible for humoral and cellular immunity, respectively. Most are small lymphocytes 7-10 μm in diameter with a round or slightly indented heterochromatic nucleus that almost fills the entire cell and a thin rim of basophilic cytoplasm that contains few granules. When “activated” by contact with antigen, small lymphocytes begin macromolecular synthesis, the cytoplasm enlarges until the cells are 10-30 μm in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

‘Innate immunity’ refers to immune responses found in all classes of plants and animals that provide immediate defense against pathogens, and also immune responses that are triggered at sites of infection.

‘Adaptive immunity’ refers to responses of the vertebrate immune system that provide specific and long-lasting protection against a particular antigen, also referred to as immunological memory, in peripheral lymphoid organs. As innate and adaptive immunity are interrelated, certain types of lymphocytes partake in both these systems.

In some embodiments, the lymphocyte cell modulated by the modulator used by the invention may be at least one of an NK cell, a T cell and a B cell forming an inhibitory or activating IS. For example, natural killer (NK) cells are a type of cytotoxic lymphocytes that are critical to the innate immune system in providing rapid responses to viral-infected cells and tumor formation. Further, NK cells are effectors of innate immunity in expressing activating and inhibitory NK receptors, which play an important function in self-tolerance and in sustaining NK activity. Killer-cell immunoglobulin-like receptors (KIRs), are a family of type I transmembrane glycoproteins expressed on the plasma membrane of natural killer (NK) cells and a minority of T cells. They regulate the killing function of these cells by interacting with major histocompatibility (MHC) class I molecules, which are expressed on all nucleated cell types. KIR receptors can distinguish between major histocompatibility (MHC) class I allelic variants, which allows them to detect virally infected cells or transformed cells. Most KIRs are inhibitory, meaning that their recognition of MHC molecules suppresses the cytotoxic activity of their NK cell. Only a limited number of KIRs are activating, meaning that their recognition of MHC molecules activates the cytotoxic activity of their cell.

Natural killer cell cytolysis of target cells and cytokine production is controlled by a balance of inhibitory and activating signals, which are facilitated by NK cell receptors. NK cell inhibitory receptors are part of either the immunoglobulin-like (IgSF) superfamily or the C-type lectin-like receptor (CTLR) superfamily. Members of the IgSF family comprise the human killer cell immunoglobulin-like receptor (KIR) and the Immunoglobulin-like transcripts (ILT).

Inhibitory receptors recognize self-MHC class I molecules on target self cells, causing the activation of signaling pathways that stop the cytolytic function of NK cells. Self-MHC class I molecules are always expressed under normal circumstance. According to the missing-self hypothesis, inhibitory KIR receptors recognize the downregulation of MHC class I molecules in virally-infected or transformed self cells, leading these receptors to stop sending the inhibition signal, which then leads to the lysis of these unhealthy cells. Because natural killer cells target virally infected host cells and tumor cells, inhibitory KIR receptors are important in facilitating self-tolerance.

KIR inhibitory receptors signal through their immunoreceptor tyrosine-based inhibitory motif (ITIM) in their cytoplasmic domain. When inhibitory KIR receptors bind to a ligand, their ITIMs are tyrosine phosphorylated and protein tyrosine phosphatases, including SHP-1, are recruited.

Activating receptors recognize ligands that indicate host cell aberration, including induced-self antigens (which are markers of infected self cells and comprise MICA, MICB, and ULBP, all of which are related to MCH class 1 molecules), altered-self antigens (MHC class I antigens laden with foreign peptide), and/or non-self (pathogen encoded molecules). The binding of activating KIR receptors to these molecules causes the activation of signaling pathways that cause NK cells to lyse virally infected or transformed cells.

Activating receptors do not have the immunoreceptor tyrosine-base inhibition motif (ITIM) characteristic of inhibitory receptors, and instead contain a positively charged lysine or arginine residue in their transmembrane domain (with the exception of KIR2B4) that helps to bind DAP12, an adaptor molecule containing a negatively charged residue as well as immunoreceptor tyrosine-based activation motifs (ITAM). Activating KIR receptors include KIR2DS, KIR2DL1, and KIR3DS.

NK cells express receptors for MHC class I molecules comprising the C-type lectin-like receptors, CD94/NKG2. CD94/NKG2 receptors are expressed on majority of NK cells and a subset of CD8+ T cells. Five different molecular species of NKG2 (NKG2A, B, C, E and H) have been reported to form disulfide-linked heterodimers with invariant CD94. NKG2A and B, which are products from a single gene by alternative splicing, have two immunoreceptor tyrosine-based inhibitory motifs (ITIM) in their cytoplasmic domains and form inhibitory receptors complexed with CD94. NKG2C, E and H, the latter two of which are also products from a single gene, as well as NKG2C, have positively charged residues within their transmembrane regions. NKG2C and possibly NKG2E and H interact with the adapter molecule DAP12, and act as activating receptors, when heterodimerized with CD94.

NK cells also play a role in adaptive immune response in their ability to readily adjust to the immediate environment and formulate antigen-specific immunological memory, fundamental for responding to secondary infections with the same antigen. Thus NK cells are acting in both the innate and adaptive immunity, which makes them particularly useful targets for modulators of the present invention. This particular feature of NK cells has been corroborated on various levels by the present EXAMPLES 1-7. Thus, in some embodiments, the invention provides modulators of ARF for use in modulating the activation of NK cells.

In the context of adaptive immunity, it is further contemplated that T cell lymphocytes, including cytotoxic T cells (CTLs) and helper T cells, are also liable to modulation according to the present invention. CTLs kill infected cells, whereas helper T cells help activate macrophages, B cells, and CTLs. Of further relevance are various types of effector T cells, i.e. T cells activated by their cognate antigen. Upon antigen activation, the effector T helper cells secrete a variety of cytokines and display a variety of membrane-bound costimulatory proteins, by which they can influence the behavior of the various neighboring cells. The effector CTLs kill infected target cells by means of proteins that they either secrete or display on their surface. NK cells morphologically differ from CTLs, as well as by origin and effector functions. Often, CTL activity promotes NK activity by secreting IFNγ. In contrast to CTLs, NK cells do not express T cell antigen receptors (TCR) or pan T marker CD3 or surface immunoglobulins (Ig) B cell receptors, instead they express the surface markers CD16 (FcγRIII) and CD56 in humans (NK1.1 or NK1.2 in mice), up to 80% of human NK cells also express CD8.

In yet some further embodiments, the ARF modulators may be used by the invention for modulating the activation of T cells forming inhibitory or activating IS.

In some further embodiments, the ARF modulators may be used by the invention for modulating the activation of B cells forming inhibitory or activating IS.

This invention may be further articulated from the point view of a lymphocyte forming an immunological synapse (IS) as the target for the presently conceived modulators. The IS model in relating to features such as T cell membrane structure, T cell polarity, signaling pathways, and antigen-presenting cells (APC), provides a comprehensive view on T cells maturation and activation. Originally the term ‘IS’ denoted a crucial junction between a T cell and APC at which T cell receptors (TCRs) interact with MHC molecules. Today however this term has been expended to include different types of immune cells, as well as non-immune cells. Thus, the term ‘IS’ herein denotes a specific arrangement of molecules in an immune cell at the interface with another cell. Molecules related to IS formation may include, although not limited to, receptors, signaling molecules, cytoskeletal elements and cellular organelles. When referring to arrangement of said molecules is meant, for example, accumulation of molecules in distinct regions within an activating IS to form a supramolecular activation cluster (SMAC), which may be further segregated into peripheral (pSMAC) and central (cSMAC) zones. This term further encompasses other features, such as engagement of individual receptors, or involvement of microclusters of cell-surface, and signaling molecules that support cell activation and maturation of IS.

The process of IS formation can be further described in a sequential manner, initially causing a significant large-scale redistribution of a number of integral membrane and cytosolic proteins. At the T cell/APC interface the structure comprises in its nascent stage a non-random pattern of protein distribution. The protein pattern is regulated during development of the mature IS and is finally organized into concentric rings of co-receptors and adhesive molecules surrounding TCR. The relocations of proteins are influenced by passive as well as active mechanisms.

The IS model was originally denoted the interaction between a T helper cell and APC, but may also apply to the NK cell IS (NKIS). Certain aspects are particularly relevant to NK cells, such as directed secretion of lytic granules for cytotoxicity. This model when applied to NK cell activation is especially informative for the inhibitory NKIS, which a striking example wherein inhibition of signaling leaves the synapse in its nascent, inverted state (early stage). Utility of the modulators of the present invention for activation of the nascent NKIS has been corroborated by EXAMPLE 7.

Thus in specific embodiments, the modulators according to the present invention are particularly applicable to a lymphocyte cell that is a NK cell forming an activating or inhibitory NK immunological synapse (NKIS). More specifically, Natural killer cells or NK cells are a type of cytotoxic lymphocyte critical to the innate immune system. NK cells (belonging to the group of innate lymphoid cells) are defined as large granular lymphocytes (LGL). The role NK cells play is analogous to that of cytotoxic T cells in the vertebrate adaptive immune response. NK cells provide rapid responses to viral-infected cells, acting at around three days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. Still further, in a broader sense, ‘NKIS’ denotes the dynamic interface formed between an NK cell and its target cell. Formation of NKIS involves several distinct stages, beginning with the initiation of contact with a target cell and culminating in the directed delivery of lytic granule contents to lyse the target cell. Progression through the individual stages is methodical and underlies the precision with which NK cells select and kill susceptible target cells (including virally infected cells and cancerous cells) that they encounter during their routine surveillance of the body.

More specifically, the formation of a mature and functional NKIS can be divided into a series of sequential (nonparallel) stages: the recognition and initiation stage, the effector stage and the termination stage. Together, these processes enable the delivery of lytic granules to the synapse followed by their close association with the NK cell membrane to which they can fuse and release their contents onto the target cell. Because lytic granules exist in resting NK cells before activation, each stage must be controlled to prevent accidental release of cytotoxic mediators and to enable rapid directed secretion at the appropriate moment. Of particular relevance are molecules related to the above processes, which can be used as markers for evaluating activity of the modulators of the present invention. Specifically:
The initial stage is characterized by formation of a close association between the NK cell and a target cell, initial signaling and adherence of NK cell to its target cell. This stage is facilitated by a number of molecules, including, although not limited to, members of the selectin family, the CD2 receptor, and receptors from the integrin family of adhesion molecules in particular such as the integrins lymphocyte function-associated antigen 1 (LFA1; CD11a/CD18) and MAC1 (CD11b/CD18). Importantly, this initial stage is rapid and occurs before molecular patterning is evident. The decision whether NK cell progresses to maturation and molecular reorganization at NKIS depends on the level of signals through inhibitory receptors (KIRs, Killer-cell Immunoglobulin-like Receptors), which can establish a so-called inhibitory synapse. Such regulation ensures that NK cells effectively carry out their surveillance function, by leaving most cells undisturbed, while being poised to destroy those that are diseased. The inhibitory NKIS is especially elegant in that it directly interferes with the ability of the lytic synapse to progress past the initiation stage.
The effector stage, which is probably the most relevant to the present invention, is characterized by a number of processes, most prominent of which are (1) formation of a stable NK cell-target cell interface with a ‘cleft’ into which cytolytic molecules are secreted; (2) recruitment of lytic granules to the synapse; (3) clearance of a conduit in the NK cell cortex through which lytic granules could be directed to the cell membrane; and (4) fusion of the lytic-granule membrane with plasma membrane for release of lytic-granule contents. The effector function has been particularly related to actin reorganization at NKIS, which occurs downstream of activating-receptor-induced VAV1 (guanine nucleotide exchange factor) and WASP (Wiskott-Aldrich syndrome protein) activities. Parallel events include receptor clustering, lipid-raft aggregation, further activation signaling and lytic-granule redistribution. Among receptors that undergo clustering, the most important for both, adhesion and triggering of cytotoxicity, are CD11a, CD11b and CD2. Receptors clustering has been also identified in T cells forming IS, wherein TCR signaling was traced to microclusters of TCR molecules that move from the pSMAC into the cSMAC as activation proceeds. Another requirement for effector function is polarization of lytic granules to NKIS, or in other words movement of the granules along the microtubules to the microtubule-organizing centre (MTOC). Signals required for MTOC polarization include ERK (extracellular-signal-regulated kinase) phosphorylation, VAV1 activation and PYK2 (protein tyrosine kinase 2) activities. Certain members of the PLC/PKC pathways and VAV1 in particular were presently demonstrated as useful markers for evaluating activity of several typical modulators of the present invention (see EXAMPLE 6). It should be further noted that in T cells MTOC polarization requires CDC42 (cell-division cycle 42) and activation of a signaling platform comprising ZAP70 (ζ-chain-associated protein kinase of 70 kDa), SLP76. Still further, granule docking to the synapse requires members of the RAB family of small GTPases, which are important regulators of vesicle trafficking and compartmentalization. RAB27a also performs this function in docking lytic granules in CTLs. Of further relevance are Munc13-4 (putative vesicle priming factor) and SNAREs (N-ethylmaleimide-sensitive fusion protein attachment protein receptors) and their regulators acting in a coordinated manner to facilitate membrane fusion and providing a fine-tuning of NKIS. One of the important findings by the present inventors is that SHP-1 (a SH2 domain containing tyrosine phosphatase), yet another regulator of the effector function, is a useful marker for evaluating activity of the modulators of the invention in being directly affected by the actin network dynamics and ARF (see EXAMPLES 5-6). A detailed discussion on this regulator of the effector function and its relevance to the present invention follows further below.
Termination stages of NKIS refer to those that occur after the lytic-granule contents have been secreted. Those include a period of inactivity and down modulation of the accumulated activating receptors followed by NK cell detachment from the target cell and recycling of cytolytic capacity. In T cells this is achieved by TCR internalization via the cSMAC and further includes localized TCR ubiquitylation. Once NK cell has carried out its cytolytic function, it can detach from the target cell and restore its ability to kill another susceptible cell. At the inhibitory synapse, detachment may result from reduced integrity of interactions between the F-actin cortex and the plasma membrane through dephosphorylation of ERM protein targets. The signals initiating the process of recycling NK cytolytic capacity are largely unknown, apart from activation of the nuclear factor-KB (NF-κB) which has been shown to serve as a transcription factor for expression of the lytic granule component perforin.

As mentioned above, the inhibitory NKIS, wherein the synapse is in its nascent state, is of a particular interest for the present invention, as it is conceived. Thus, in specific embodiments, the modulators of the invention may be capable activating NK cells in inhibitory NKIS by inhibiting ARF in said NK cells. More specifically, in some embodiments the invention provide the use of ARF modulators that inhibit and thereby disturbs ARF in NK cells. Such ARF inhibitors are used by the invention for activating NK cells that form inhibitory NKIS.

As noted above, the invention provides ARF modulators for use in modulating the activation of hematopoietic cells. Thus, in certain embodiments, in addition to modulation in the activation of lymphocytes, and specifically, NK cells, the modulators of the invention may be used in modulating the activation of other hematopoietic cells. In further embodiments, the modulators of the invention may upregulate platelet and/or megakaryocyte activation. In yet some other embodiments, the modulators of the invention may activate platelet/s and/or megakaryocyte/s as manifested by at least one of cell spreading, cell aggregation, elevation in intracellular calcium concentration, cell adhesion, phagocytosis, and cytolytic activity.

A megakaryocyte is a large bone marrow cell with a lobulated nucleus responsible for the production of blood thrombocytes (platelets), which are necessary for normal blood clotting. Megakaryocytes are derived from hematopoietic stem cell precursor cells in the bone marrow. The “platelet/s” as used herein, are one of the key elements of human blood, playing a central role in the process of thrombus formation. The main function of platelets is the formation of mechanical plugs during the normal hemostatic response to the vessel wall injury. Platelets are derived from the megakaryocytes in the bone marrow. These megakaryocytes arise by a process of differentiation from the haemopoietic stem cell and undergo fragmentation of their cytoplasm to produce platelets. Platelet production is under the control of humoral agents such as thrombopoietin. The platelet is an enucleate cell that beside nucleus includes intracellular organelles in the cytoplasm. Resting platelets are discoid and have a smooth, rippled surface. The platelet surface has various receptors to which various stimulants (agonists) bind and thereby activate platelets producing changes within the platelet as well as a change in platelet shape from discoid to spherical, adhesion and aggregation of platelets. One of the methods to evaluate “platelet activation” in response to agonists is by measuring intracellular calcium concentration. Another method is to quantify platelet release products in the plasma. More specifically, resting platelets maintain active calcium efflux via a cyclic AMP activated calcium pump. Intracellular calcium concentration determines platelet activation status, as it is the second messenger that drives platelet conformational change and degranulation. Platelet activation begins seconds after adhesion occurs. Thrombin is a potent platelet activator. Thrombin also promotes secondary fibrin-reinforcement of the platelet plug. Platelet activation in turn degranulates and releases factor V and fibrinogen, potentiating the coagulation cascade. Following their activation and F-actin polymerization, platelets must spread over intact blood vessels in the process of clot formation. Adhesion of platelets to fibrinogen is a key process in platelet aggregation, mediated by integrins, such as αIIbβ3. Platelets contain dense granules, lambda granules and alpha granules. Activated platelets secrete the contents of these granules through their canalicular systems to the exterior.

In yet some further embodiments, the modulators of the invention may affect any non-erythroid hematopoietic cell, for example, any one of Granulocytes, neutrophils, Eosinophils, Basophils, Monocytes, Macrophages, and Dendritic cells (DCs).

More specifically, Granulocytes are a category of white blood cells characterized by the presence of granules in their cytoplasm. They are also called polymorphonuclear leukocytes (PMN, PML, or PMNL) because of the varying shapes of the nucleus, which is usually lobed into three segments. This distinguishes them from the mononuclear granulocytes.

Neutrophils are normally found in the bloodstream and are the most abundant type of phagocyte, constituting 50% to 60% of the total circulating white blood cells. Once neutrophils have received the appropriate signals, it takes them about thirty minutes to leave the blood and reach the site of an infection. Neutrophils do not return to the blood; they turn into pus cells and die. Mature neutrophils are smaller than monocytes, and have a segmented nucleus with several sections (two to five segments); each section is connected by chromatin filaments. Neutrophils do not normally exit the bone marrow until maturity, but during infection neutrophil precursors called myelocytes and promyelocytes are released.

Neutrophils display three strategies for directly attacking micro-organisms: phagocytosis (ingestion), release of soluble anti-microbials (including granule proteins), and generation of neutrophil extracellular traps (NETs). The intracellular granules of the human neutrophil have long been recognized for their protein-destroying and bactericidal properties.

Eosinophils play a crucial part in the killing of parasites (e.g., enteric nematodes) because their granules contain a unique, toxic basic protein and cationic protein (e.g., cathepsin). These cells also have a limited ability to participate in phagocytosis, but are professional antigen-presenting cells. They are able to regulate other immune cell functions (e.g., CD4+ T cell, dendritic cell, B cell, mast cell, neutrophil, and basophil functions) and are involved in the destruction of tumor cells. In addition, they promote the repair of damaged tissue.

Basophils are one of the least abundant cells in bone marrow and blood. The cytoplasm of basophils contains a varied amount of granules; these granules are usually numerous enough to partially conceal the nucleus. Granule contents of basophils are abundant with histamine, heparin, chondroitin sulfate, peroxidase, platelet-activating factor, and other substances. When an infection occurs, mature basophils will be released from the bone marrow and travel to the site of infection. When basophils are injured, they will release histamine, which contributes to the inflammatory response that helps fight invading organisms. Mast cells also contain many granules rich in histamine and heparin. Although best known for their role in allergy and anaphylaxis, mast cells play an important protective role as well, being intimately involved in wound healing, angiogenesis, immune tolerance, defense against pathogens, and blood-brain barrier function. The mast cell is very similar in both appearance and function to the basophil.

Monocytes are a type of white blood cell, or leukocyte. They are the largest type of leukocyte and can differentiate into macrophages and myeloid lineage dendritic cells.

Macrophages are a type of white blood cell that engulfs and digests cellular debris, foreign substances, microbes, cancer cells, and anything else that does not have the types of proteins specific to healthy body cells on its surface in a process called phagocytosis. These large phagocytes are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in nonspecific defense (innate immunity) and also help initiate specific defense mechanisms (adaptive immunity) by recruiting other immune cells such as lymphocytes. For example, they are important as antigen presenting cells to T cells.

Dendritic cells (DCs) as used herein are antigen-presenting cells (also known as accessory cells) of the mammalian immune system. Their main function is to process antigen material and present it on the cell surface to the T cells of the immune system. They act as messengers between the innate and the adaptive immune systems. Dendritic cells are present in the skin (where there is a specialized dendritic cell type called the Langerhans cell) and the inner lining of the nose, lungs, stomach and intestines. Once activated, DCs migrate to the lymph nodes where they interact with T cells and B cells to initiate and shape the adaptive immune response.

In further embodiments, the activity of this type of modulators, i.e. those acting via the ARF inhibition, results in formation of a complex comprising β-actin and at least one phosphatase, specifically, at least one protein tyrosine phosphatase (PTP). In some specific embodiments, this PTP may be a Src homology region 2 (SH2) domain-containing phosphatase in the NK cell. The existence of such complex and its functional implications for modulation of ARF in NK cells in particular has been strongly supported by the present EXAMPLES.

In some specific embodiments, application of the modulators of the invention may result in formation of a complex comprising at least one of said PTPs and β-actin. In yet some further embodiments, the use of ARF inhibitors may result in at least one of, change in the conformation of at least one PTP and change in the catalytic activity of said PTP in the NK cell.

The term ‘actin’ has been articulated herein in various contexts. In the above described aspects and embodiments it was used to convey actin functionality and structural meaning. In most general terms, ‘actin’ is a ubiquitous globular protein that is one of the most highly-conserved proteins known. Structurally, the term ‘actin’ refers to the two main states of actin: the G-actin—the globular monomeric form and the F-actin forming helical polymers. Both G- and F-actin are intrinsically flexible structures—a feature vital in actin's role as a dynamic filament network. In terms of functionality, the F-actin polymers form microfilaments—polar intracellular ‘tracks’ for kinesin motor proteins, allowing the transport of vesicles, organelles and other cargo. Further, actin is a component of the cytoskeleton and links to alpha-actinin, E-cadherin and beta-catenin at adherens junctions. This gives mechanical support to cells and attaches them to each other and the extracellular matrix. In muscle cells, actin-rich thin filaments associate with myosin-rich thick filaments to form actomyosin myofibrils. Using energy from the hydrolysis of ATP, myofibrils undergo cyclic shortening through actin-myosin head interactions, which represents the mechanics of muscle contraction. Finally, actin has a role in cell motility through polymerization and depolymerization of fibrils.

When now referring to the ‘beta-actin’ is meant the product(s) of the actin gene. In specific embodiments that are applicable to humans, this term refers to the human gene and protein symbol ACTB, which is one to the six different actin isoforms identified in humans. This actin is a major constituent of the contractile apparatus and one of the two nonmuscle cytoskeletal actins. More specifically, when referring herein to the human beta-actin gene product(s) is meant in some embodiments, the product (s) of the ACTB gene (also BRWS1, PS1TP5-binding protein 1 (PS1TP5BP1), Beta Cytoskeletal Actin, Cytoplasmic Actin 1) located at the human chromosome 7p22.1. In some specific embodiments, beta-actin as used herein refers to the human protein P60709-ACTB_HUMAN (UniProtKB/Swiss-Prot), RefSeq protein: NP_001092.1. More specifically, in some embodiments, such protein may comprise the amino acid sequence as denoted by SEQ ID NO 5, having 375 amino acids, and molecular mass of approximately 42 kDa. In yet some further embodiments, beta-actin may be encoded by a cDNA molecule denoted by accession number NM_001101.4, specifically, and may comprise the nucleic acid sequence as denoted by SEQ ID NO:4.

Still further, in some embodiments, the ARF modulators, specifically, ARF inhibitors used by the invention, by inhibiting ARF, lead to formation of a complex comprising β-actin and at least one Src homology region 2 (SH2) domain-containing phosphatase (SHP). The terms ‘Src homology region 2 (SH2) domain-containing phosphatase’ (also Src tyrosine kinase activating tyrosine phosphatases) or simply SH2 domain-containing phosphatases refer to the most studied classical non-receptor tyrosine phosphatases (also non-receptor protein tyrosine phosphatases, PTPNs), SHP-1 and SHP-2. Both these phosphatases are characterized in that they possess a domain structure that consists of two tandem SH2 domains in its N-terminus followed by a PTP domain. This particular structure of the N-terminal of SHP-1 and SHP-2 is unique among other proteins with SH2 domains and confers them switching or auto-inhibiting property.

Further, the SHP-1 and SHP-2 phosphatases, sharing close sequence and structural homology. The major sequence differences between these two proteins are apparent in the approximately 100 amino acid residues at the extreme C-terminus, beyond the phosphatase catalytic domain Thus when referring herein to the SH2 domain containing tyrosine phosphatases is meant the entire family of these proteins, including the four isoforms of SHP-1 and one isoform of SHP-2, and further the hematopoietic and non-hematopoietic cell specific isoforms of SHP-1, the latter arising from an alternative initiation site and varying by three amino acids at the N-terminus. This family further includes the longer 70 kDa form of SHP-1 (SHP-1L) that differs by 66 amino acids at the C-terminus due to alternative splicing of SHP-1 transcripts and subsequent shift of the reading frame. Relative to SHP-1, SHP-1L lacks one of the tyrosine phosphorylation sites and has a Pro-rich motif, a putative SH3-domain binding motif. As mentioned above, SHP-1 and SHP-2 share a characteristic N-terminal SH2 domain with phosphotyrosine binding sites facing outwards, which confer them the ability of auto-regulating phosphatase activity. The C-terminal SH2 domain has little interaction with the N-terminal SH2 domain or the catalytic domain.

More specifically, in the ‘closed’ inhibited form (also inactive or ‘I’ state) the N-terminal SH2 domain forms extensive contacts with the catalytic domain through charge-charge-interactions, namely a part of the SH2 domain, the NXGDY/F motif, is inserted into the catalytic cleft of the enzyme, thus blocking access of substrates to the active site. Upon binding of a phosphopeptide, such as beta actin for example, the N-terminal SH2 domain undergoes an allosteric switch from the inactive ‘I’ state to the active ‘A’ state. This conformational change in the N-terminal SH2 domain disrupts the interaction between the SH2 domain and the phosphatase domain, and allows access of substrates.

It should be appreciated that conformational changes as described herein after in connection with SHP-1, as a result of interaction thereof with actin, refer to changes from “closed” to “opened” conformation and vice versa.

The terms ‘SHP-1’ and ‘SHP-2’ refer herein to these genes product(s). In specific embodiments that are applicable to humans, these genes are denoted as, for SHP-1 as Protein Tyrosine Phosphatase Non-Receptor Type 6 (PTPN6), Hematopoietic Cell Protein-Tyrosine Phosphatase (HCP), Protein-Tyrosine Phosphatase 1C (PTP-1C, HPTP1C), SH-PTP1, and EC 3.1.3.48; and for SHP-2 as Protein Tyrosine Phosphatase, Non-Receptor Type 11 (PTPN11), Protein-Tyrosine Phosphatase 1D (PTP-1D), Protein-Tyrosine Phosphatase 2C (PTP2C), SH-PTP2, SH-PTP3 and EC 3.1.3.48; and further as the SHP-1 gene is located at the human chromosome 12p13.31 and the SHP-2 gene located at the human chromosome 12q24.13. These terms are applied herein to all known transcription isoforms and post translational modifications, such phosphorylation and ubiquitinations at specific residues.

In some embodiments, the human PTPN6 cDNA may refer to any one of transcript variant 1 NM_002831.5, that comprises the nucleic acid sequence as denoted by SEQ ID NO:6, PTPN6 cDNA, transcript variant 2 NM_080548.4, that comprises the nucleic acid sequence as denoted by SEQ ID NO:13 and PTPN6 cDNA, transcript variant 3 NM_080549.3, that comprises the nucleic acid sequence as denoted by SEQ ID NO:14. In yet some further specific embodiments, the protein P29350-PTN6_HUMAN (SHP-1) referred to herein, may include the following isoforms (UniProtKB/Swiss-Prot), as denoted by RefSeq NP_002822.2 as denoted by SEQ ID NO 7, NP_536858.1 as denoted by SEQ ID NO 8, NP_536859.1 as denoted by SEQ ID NO 9, respectivelly. Thus, in some embodiments, the modulators of the invention are used to modulate the interaction of bata-actin with SHP-1 or any variants thereof, specifically, as disclosed herein. Still further, the human PTPN11 cDNA may refer to any one of transcript variant 1, cDNA NM_002834.4, that comprises the nucleic acid sequence as denoted by SEQ ID NO 10 and PTPN11 transcript variant 2, cDNA NM_080601.2, that comprises the nucleic acid sequence as denoted by SEQ ID NO 15; and the protein Q06124-PTN11_HUMAN (UniProtKB/Swiss-Prot), and RefSeq NP_002825.3 as denoted by SEQ ID NO 11, NP_542168.1 as denoted by SEQ ID NO 12, respectively, having 597 amino acids and molecular mass of approximately 68.5 kDa. It should be appreciated that in some embodiments, the modulators of the invention are used to modulate the interaction of bata-actin with SHP-2 or any variants thereof, specifically, as disclosed herein.

Still further, in some embodiments, the invention provides ARF modulators for use in the modulation of the conformation and/or catalytic activity of at least one of SHP-1 and SHP-2 in a cell.

The relevance of SHP-1 to the present invention has been extensively corroborated on several levels: first on the level of formation of the β-actin:SHP-1 complex (see EXAMPLE 1) and further on the level of ARF regulation of SHP-1 conformation and catalytic activity (EXAMPLE 5), including phosphorylation of its natural substrates VAV1 and PLCγ1/2 (EXAMPLE 6). The modulators of the present invention have been demonstrated to act via this particular mechanism in activating cytotoxic potential of NK cells forming inhibitory NKIS, as revealed in increased intracellular Ca²⁺ flux, and secretion or formation of cytolytic granules in those cells (see EXAMPLE 7 and FIG. 13).

Thus, in some specific embodiments, the modulators according the present invention are acting via the SH2 domain-containing phosphatase-1 (SHP-1), which results in the β-actin:SHP-1 complex inducing a change in the SHP-1 conformation and catalytic activity in the NK cell.

It should be appreciated that the present invention describes a novel pathway of NK cell inhibition, demonstrating that in the early stages of the inhibitory NKIS, actin network dynamics play an active role in dictating SHP-1 enzymatic activity, resulting in dephosphorylation of VAV1 and PLCγ1/2. The actin network and, specifically, ARF, serves as master regulator of inhibitory signals, thereby regulating NK cell activation threshold. More specifically, the inventors demonstrate that ARF regulates the conformation of the phosphatase, SHP-1, a key enzyme in mediating the inhibitory NK cell response. By discovering a direct molecular interaction between β-actin and SHP-1 and the effect of ARF arrest in reducing SHP-1 activity, the invention demonstrates that the changes in PLCγ activation and intracellular calcium flux in NK cells are a consequence of the effect of ARF on SHP-1 conformation and activity. It is possible that a similar mechanism operates in other cell types, and possibly on other enzymes.

Still further, SHP-1 is expressed in hematopoietic cells (high level of expression) but also in epithelial cells (low level of expression). Therefore, in some embodiments modulators of the invention may be those that have an effect on the structure and the catalytic activity of SHP-1 via ARF, and transform a cell from an inactive state to an active one, and vice et versa.

Still further, ARF suppression induces elevation of the tyrosine phosphorylation of VAV1 and PLCγ1/2 at the inhibitory NKIS, indicating that ARF regulation of SHP-1 activity controls the activation of key signaling events in NK cells. Indeed, blocking of actin centripetal flow resulted in elevated intracellular calcium flux during the NK inhibitory response, which is consistent with the enhanced PLCγ1/2 phosphorylation observed. Most importantly, blocking of ARF increased the secretion of lytic granules and cytotoxicity by NK cells toward inhibitory target cells. This indicates that the ARF plays a key role in downregulating cytotoxicity at the inhibitory NKIS, and in the conversion of an inhibitory NKIS into an activating one. Altogether, the data presented by the invention reveal that the behavior of F-actin dynamics at the NKIS site, and specifically ARF, distinguishes between the NK inhibitory and activating responses by controlling the conformational structure and activation status of a key signaling molecule, thereby modulating NK cell cytotoxicity.

Thus, in certain embodiments, activation of NK cells by the ARF modulators, specifically, inhibitors, used by the invention may result in phosphorylation of at least one of VAV1 and PLCγ1/2 and/or increase in intracellular calcium flux.

Thus, in yet some further embodiments, the invention provides ARF modulators for use in methods for modulating the phosphorylation of VAV1. In yet some further specific embodiments, the invention provides the use of ARF inhibitors for increasing the phosphorylation of VAV1. VAV1 as used herein, is a protein encoded by this proto-oncogene is a member of the Dbl family of guanine nucleotide exchange factors (GEF) for the Rho family of GTP binding proteins. VAV1 plays a role in T-cell and B-cell development and activation. VAV1 activates small GTPase proteins of the Rho family, and is essential for actin reorganization and lytic granule polarization towards the target cells.

In yet some further embodiments, the invention provides ARF modulators for use in methods for modulating the phosphorylation of PLCγ1/2. In yet some further specific embodiments, the invention provides the use of ARF inhibitors for increasing the phosphorylation of PLCγ1/2.

Phosphoinositide phospholipase C (PLC) is a family of eukaryotic intracellular enzymes that play an important role in signal transduction processes and act as phosphodiesterase (PDE). PLCγ (120-155 kDa) is activated by receptor and non-receptor tyrosine kinases due to the presence of two SH2 and a single SH3 domain situated between a split PH domain within the linker region. Although this particular isoform does not contain classic nuclear export or localization sequences, it has been found within the nucleus of certain cell lines. There are two main isoforms of PLCγ expressed in human specimens, PLC-γ1 and PLC-γ2. These proteins are responsible for the release of Ca²⁺ from the endoplasmic reticulum, crucial for NK cell effector functions, namely cytokine production and cytotoxicity.

In numerous embodiments, the modulators used according to the present invention are activating NK cells forming inhibitory NKIS which results in an increase in at least one of intracellular Ca²⁺ flux, and secretion of cytolytic granules in said NK cell. The invention therefore provides in some embodiments thereof inhibitors of ARF for use in activating NK cells forming inhibitory NKIS.

As mentioned above, secretion of cytolytic granules in response to increased intracellular Ca²⁺ flux is characteristic of ‘Termination stage’ of a lymphocyte responding to an activating stimulus at IS, a which is common to NK cells and CTLs, i.e. innate or adaptive immune response. When CTL or NK cells kill infected or cancerous cells they secrete cytolytic proteins (perforin and granzymes) into the target cell. These “death factors” are pre-stored in cytolytic granules within the CTL until an increase in the intracellular Ca2⁺ drives granule to exocytosis. Secretion of cytolytic granules and increased intracellular Ca²⁺ flux are measurable (see for example FIGS. 12A-12E) and can serve as markers for evaluating the activity of the presently conceived modulators.

According to the presently introduced inventive concept the main feature of the modulators of the invention is manifested in their ability to affect ARF or actin and/or myosin dynamics in lymphocytes cells. One structural requirement of such modulators is a reduced upper molecular weight limit to allow rapid diffusion thereof across cell membranes, so that they can reach intracellular sites of action. Thus, in some embodiments, the invention provides ARF modulators for use in modulation of lymphocyte activation, that may be small molecules. In molecular biology and pharmacology, under the term ‘small molecule drug’ (SMD) is meant a biologically active low molecular weight organic compounds, characterized as having molecular weight up to 900 daltons and a size in the order of 10 nm. Most drugs are SMDs. It is thus conceived that the molecular weight of the modulators of the invention may be in the range of at least about 1-1000 daltons, or 100-900 daltons, or 200-800 daltons, or 300-700 daltons, or 400-600 daltons. It is further conceived that such modulators in the range of 1-10 nm, or 1-20 nm, or 1-30 nm, or 1-40 nm or 1-50 nm.

SMDs are more likely to be absorbed, although some of them are only absorbed after oral administration if given as prodrugs. One advantage that SMDs have over ‘large molecule’ biologics (mainly peptides and proteins), is that they can be designed as metabolically stable and orally active can be taken orally. Certain kinds of SMD, such as Jasplakinolide (JAS), the Rho kinase inhibitor of myosin light chain (MLC) phosphorylation (Y-27632) and actin-targeting SMDs, such as Cytochalasin D that are presented herein as non-limiting examples, have been exemplified as competent modulators of ARF in NK cells forming inhibitory NKIS in particular. Methods for identifying additional SMDs having the same or more superior capabilities have been presently demonstrated (see EXAMPLE 8). Delivery of such SMDs using nanoparticles has been presently exemplified in peripheral blood lymphocytes (PBLs) as well as NK cells (EXAMPLE 9).

Of particular relevance to the present context are SMDs inhibitors that target actin and/or myosin directly. In some embodiments the inhibitors used by the invention may include (i) inhibitors that primarily disrupt actin and/or myosin filament assembly and effectively destabilize filaments, and (ii) inhibitors that stabilize filaments and induce actin polymerization. Some non-limiting examples of SMDs belonging to both groups of inhibitors and their structures are detailed below and in Table 1.

In some specific embodiments, the ARF inhibitors used by the invention for modulating activation of lymphocytes may be cytochalasins.
Cytochalasins are among the best-known actin-targeting SMDs. These fungal metabolites bind and block the barbed end of actin filaments, thereby inhibiting polymerization and depolymerization at that end. Cytochalasins B and D are the most commonly used members of this group of molecules. Of these two, Cytochalasin D is preferable when inhibition of actin dynamics is desired because of greater selectivity for actin and lower Ki for inhibition of dynamics at the barbed end. Cytochalasin D and to a lesser extent Cytochalasin B also accelerate the ATPase activity of actin.

In yet some specific embodiments, the ARF inhibitor used by the invention for activating NK cells in inhibitory NKIS, may be Cytochalasin D. More specifically, Cytochalasin D that has the chemical formula C₃₀H₃₇NO₆, is also presented by Formula 2, disclosed by Table 1, is a member of the class of mycotoxins known as cytochalasins. Cytochalasin D is an alkaloid produced by Helminthosporium and other molds. This compound is cell-permeable and acts as a potent inhibitor of actin polymerization. It disrupts actin microfilaments and activates the p53-dependent pathways causing arrest of the cell cycle at the G1-S transition. It is believed to bind to F-actin polymer and prevent polymerization of actin monomers.

In some further embodiments, ARF inhibitors used by the invention may include Latrunculins.

Latrunculins are thiazolidinone-containing macrolides. They have advantages over Cytochalasins in studies of actin function in that they are generally more potent and appear to have a simpler and more definable mode of action. Latrunculin A, the most potent member of this family, inhibits actin polymerization, binding G-actin in a 1:1 complex, and also inhibits nucleotide exchange in the monomer. Unlike the Cytochalasins, which bind the barbed end of filaments, Latrunculin A appears to only associate with the actin monomer.

In some further embodiments, ARF inhibitors used by the invention may include Swinholide A. Swinholide A is a dimeric dilactone macrolide that binds dimers of G-actin with high affinity and has F-actin severing activity. Misakinolide A (also known as Bistheonillide A) is very similar in structure to Swinholide A, but has no filament severing activity. Instead, it caps the barbed end of filaments, although it does also bind actin dimers with an affinity equivalent to that of Swinholide A.

Still further, the inhibitors used by the invention may include Mycalolide B. Mycalolide B inhibits polymerization and induces rapid depolymerization of F-actin in vitro, apparently by severing F-actin and binding G-actin in a 1:1 complex. The activity of Mycalolide B is irreversible and appears due to covalent modification of actin by the compound. Halichondramide and Dihydrohalichondramide, which are structurally related to Mycalolide B, possess barbed-end capping and F-actin severing activity. Aplyronine A has a similar side-chain structure and mode of action to Mycalolide B. Pectenotoxin 2 and 6 sequester actin monomer with no severing or capping activity.

Structural formulas of several optional inhibitors that may be used in some embodiments as modulators for lymphocyte activation, specifically, as activators of NK cells forming an inhibitory NKIS, are disclosed in Table 1 herein below.

In yet some further embodiments, although not limiting, the invention provides the use of SMDs that stabilize actin filaments and promote actin polymerization and their structures are detailed herein below.

In some specific embodiments, as a modulator of ARF, specifically an inhibitor of ARF, the invention may use Jasplakinolide for activating NK cells in inhibitory NKIS. More specifically, Jasplakinolide (JAS), having the chemical name Cyclo[(3R)-3-(4-hydroxyphenyl)-β-alanyl-(2S,4E,6R,8S)-8-hydroxy-2,4,6-trimethyl-4-nonenoyl-L-alanyl-2-bromo-N-methyl-D-tryptophyl], of the chemical formula C₃₆H₄₅BrN₄O₆, as also denoted by Formula 12 in Table 1, is a cyclodepsipeptide isolated from a marine sponge, which induces actin polymerization, binds F-actin competitively with Phalloidin, and stabilizes actin filaments. However, unlike phalloidin, JAS readily crosses the cell membrane, not requiring permeabilization of cells with detergent or microinjection into cells for use. Utility of JAS as a modulator of ARF and NK cell activation has been corroborated by the present EXAMPLES.

In yet some further embodiments, the invention may use as a modulator phalloidin. Phalloidin, a so-called Phallotoxin from the deadly mushroom Amanita phalloides., is a bicyclic heptapeptide that binds and stabilizes actin filaments, shifting the equilibrium between G- and F-actin toward F-actin and lowering the critical concentration for polymerization.

Still further, Dolastatin 11, Hectochlorin, and Doliculide that have been recently found to induce assembly of F-actin structures, may be used by the invention, specifically, to activat NK cells in inhibitory NKIS. Dolastatin 11 and Hectochlorin, unlike JAS, are not competitive with Phalloidin for binding to F-actin. Structures of representative compounds belonging to this group of agents are shown in Table 1 herein below.

Still further, in some embodiments, of further relevance to modulation of ARF and lymphocyte activation are SMDs associated with actin functionality. One example of those is SMDs belonging to the class of tubulin- and microtubule-targeted inhibitors. Microtubles play important roles in cell motility and in interactions with and possible regulation of actin dynamics and cell polarity, either via destabilizing or stabilizing Notable examples of compounds belonging to this group of inhibitors and their structures are detailed below in Table 1.
Colchicine and Colcemid, well-known plant alkaloids that inhibit polymerization of tubulin and disrupt microtubules.
The Vinca alkaloids, Vinblastine and Vincristine are indole alkaloids found in periwinkle extracts; they are more potent microtubule-destabilizing agents than Colchicine.
Myoseverin is a synthetic 2, 6, 9-trisubstituted purine that affects morphological differentiation of muscle cells and found to induce fission of multinucleated myotubes into mononucleated fragments. Myoseverin and related derivatives are known to inhibit microtubule assembly.
Taxol (paclitaxel), a natural product from the Pacific Yew, stabilizes microtubules and induces assembly of new microtubules, leading to a reorganization of the microtubule network.
Epothilones, Eleutherobin, Discodermilide, and Laulimalide are other microtubule-stabilizing agents. Epothilones, Eleutherobin, and Discodermolide bind microtubules competitively with Taxol.

Additional SMDs and larger compounds impacting on actin functionality are contemplated as candidate modulators of the invention. Their explanation and structures are detailed in Table 1 below.

Thus, in yet some further embodiments, the invention may use as an ARF modulator, an inhibitor that may lead to activation of NK cells in inhibitory NKIS. Such inhibitors may be in some embodiments inhibitors of upstream signaling molecules.
Inhibitors of upstream signaling molecules: One example of this type of compounds are inhibitors of the myosin light chain kinase (MLCK), an upstream regulator of myosin II in both muscle and non-muscle cells. Ca²⁺/calmodulin-mediated activation of MLCK results in phosphorylation of the regulatory light chain of myosin II, resulting in increased assembly and contraction of actomyosin-based structures, such as stress fibers in non-muscle cells. A number of inhibitors of MLCK are known, among the more selective MLCK inhibitors presently available are the synthetic naphthalenesulfonamides, ML-7 and ML-9, both of which bind MLCK competitively with ATP. Another example is an inhibitor of the small GTPase RhoA that regulates myosin II in smooth muscle and non-muscle cells. GTPbound RhoA activates Rho-associated kinases (Rho-kinases), such as p160ROCK, resulting in inhibitory phosphorylation of myosin light chain phosphatase, which results in increased levels of phosphorylation of the regulatory light chain of myosin II.

In some specific embodiments, Y-27632, (C₁₄H₂₁N₃O), specifically, 4-[(1R)-1-aminoethyl]-N N-4-pyridinyl-trans-cyclohexanecarboxamide, dihydrochloride, as also presented by Formula 35 in Table 1, is a synthetic pyridine derivative that inhibits Rho-kinases, and formation of stress fibers.

In yet some other embodiments, Blebbistatin is an additional SMD inhibiting the activity of myosin. This compound is a small molecule inhibitor of nonmuscle myosin IIA. Blebbistatin potently inhibits several striated muscle myosins as well as vertebrate nonmuscle myosin IIA and IIB with IC50 values ranging from 0.5 to 5 microM. Interestingly, smooth muscle which is highly homologous to vertebrate nonmuscle myosin is only poorly inhibited (IC50=80 microM). Blebbistatin does not inhibit representative myosin superfamily members from classes I, V, and X. A further example is an inhibitor of N-WASP that links activated CDC42 and phosphatidylinositol-4,5-bisphosphate (PIP2) to induce de novo nucleation of new actin filaments and formation of filopodia. N-WASP has a known exogenous inhibitory ligand designated 187-1, which is a synthetic 14-residue cyclodimeric peptide and therefore larger than the typical SMDs. Further, synthetic oligopeptides derived from the sequence of gelsolin known to specifically bind polyphosphoinositides (PPIs) constitute another class of molecules, although again larger than typical SMDs, that can be useful reagents to probe components of actin assembly pathways.

In yet some further embodiments, inhibitors of actin-binding proteins may be also used by the invention.

Inhibitors of Actin-Binding Proteins:

Most of the cellular functions of F-actin depend not only on actin itself but also on F-actin-binding proteins, a large, functionally and evolutionarily diverse group of proteins. The main actin-binding protein for which directly binding inhibitors are known is myosin. Proteins of the myosin superfamily are ATP-hydrolyzing motors responsible for actin filament contractility in both muscle and non-muscle cells. The non-muscle myosin II bundles F-actin into antiparallel arrays and generates tension in stress fibers and other contractile actomyosin structures through ATP-dependent, barbed-end-directed motion along F-actin. One example of inhibitors of myosin II and myosin V ATPase activity is 2,3-Butanedione-2-monoxime (BDM).

Still further, in some embodiments Inhibitors of cell motility with unidentified targets may be also used: More specifically, a number of inhibitors of cell motility with as-yet unidentified cellular targets have been discovered using different whole-cell screening systems, three of which are Migrastatin, Motuporamine C, and UIC-1005.

TABLE 1
ARF modulators

| | |
|---|---|
| Cytochalasin B Formula 1 | 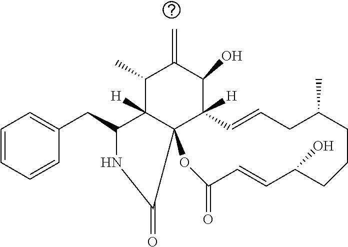 |
| Cytochalasin D Formula 2 | 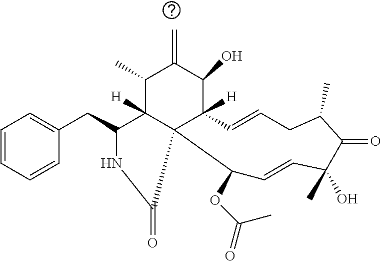 |
| Latrunculin A Formula 3 | 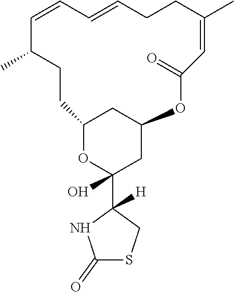 |
| Swinholide A Formula 4 | 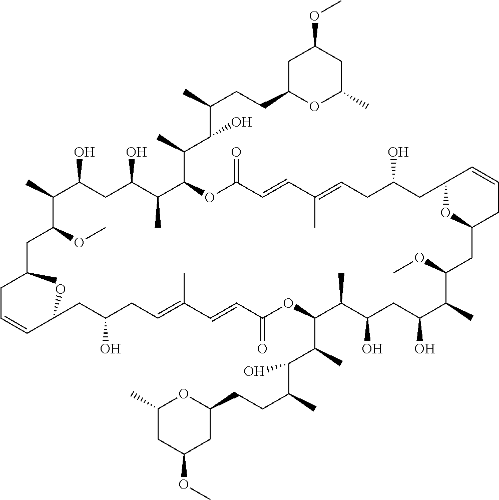 [SMILES: CO[C@@H]1C[C@H](CC[C@H](C)[C@H](O)[C@H](C)[C@H]2OC(=O)/C=C/C(C)=C/C[C@H](O)C[C@H]3C=CC[C@@H](C[C@H](OC)[C@@H](C)[C@@H](O)C[C@@H](O)[C@H](C)[C@@H]([C@@H](C)[C@@H](O)[C@@H](C)CC[C@H]4C[C@@H](OC)C[C@H](C)O4)OC(=O)/C=C/C(C)=C/C[C@H](O)C[C@H](O)/C=C\C[C@H](C)C[C@H](OC)[C@@H](C)[C@@H](O)C[C@@H](O)[C@@H]2C)O3)O[C@@H](C)C1] |
| Misakinolide_A Formula 5 | 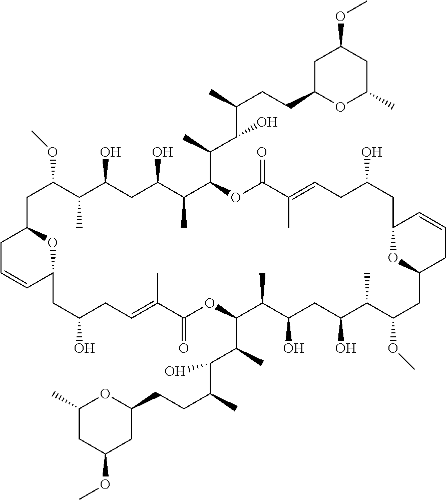 [SMILES: CO[C@@H]1C[C@H](CC[C@H](C)[C@H](O)[C@H](C)[C@H]2OC(=O)/C(C)=C/C[C@H](O)C[C@@H]3C=CC[C@@H](C[C@H](OC)[C@@H](C)[C@@H](O)C[C@@H](O)[C@H](C)[C@@H]([C@@H](C)[C@@H](O)[C@@H](C)CC[C@H]4C[C@@H](OC)C[C@H](C)O4)OC(=O)/C(C)=C/C[C@H](O)C[C@@H]4C=CC[C@@H](C[C@H](OC)[C@@H](C)[C@@H](O)C[C@@H](O)[C@@H]2C)O4)O3)O[C@@H](C)C1] |
| Tolytoxin Formula 6 | 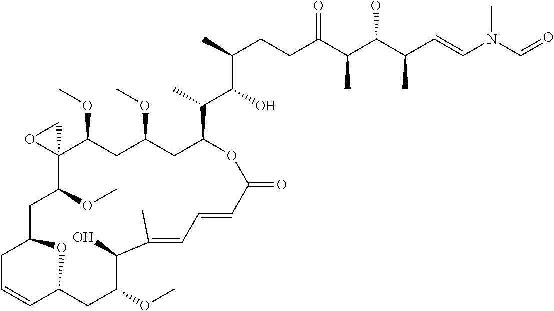 [SMILES: CO[C@H]1C[C@@H]([C@H](C)[C@@H](O)[C@@H](C)CCC(=O)[C@H](C)[C@H](O)[C@H](C)/C=C/N(C)C=O)OC(=O)/C=C/C=C(\C)[C@H](O)[C@H](OC)C[C@@H]2C=CC[C@@H](C[C@H](OC)[C@]3(CO3)[C@@H](OC)C1)O2] |
| Mycalolide B Formula 7 | 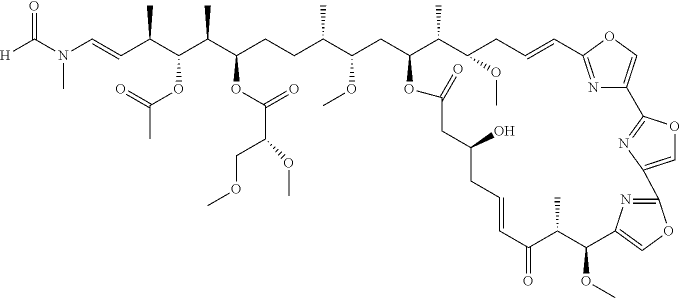 [SMILES: COC[C@@H](OC)C(=O)O[C@H](CC[C@H](C)[C@H](C[C@@H]1OC(=O)C[C@@H](O)C/C=C/C(=O)[C@H](C)[C@H](OC)c2coc(n2)-c2coc(n2)-c2coc(n2)/C=C/C[C@H](OC)[C@H]1C)OC)[C@H](C)[C@H](OC(C)=O)[C@H](C)/C=C/N(C)C=O] |
| Halichondramide Formula 8 | 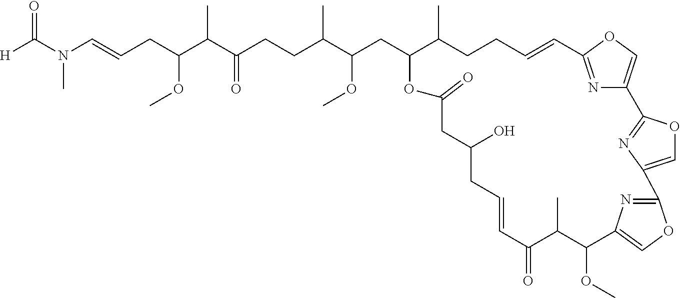 [SMILES: COC(CC1OC(=O)CC(O)C/C=C/C(=O)C(C)C(OC)c2coc(n2)-c2coc(n2)-c2coc(n2)/C=C/CCC1C)C(C)CCC(=O)C(C)C(C/C=C/N(C)C=O)OC] |
| Aplyronine A Formula 9 | 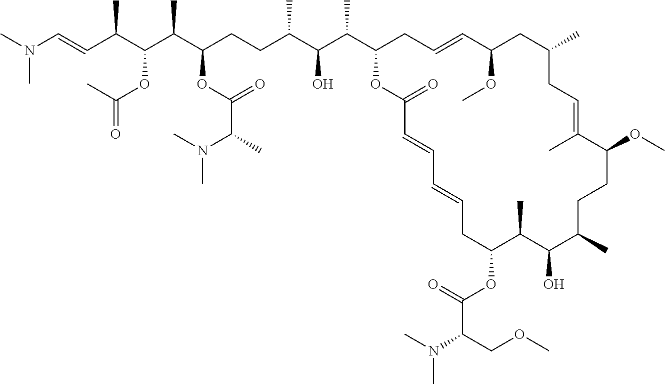 |
| Pectenotoxin 2 Formula 10 | 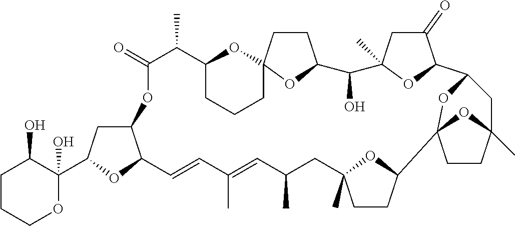 |
| Phalloidin Formula 11 | 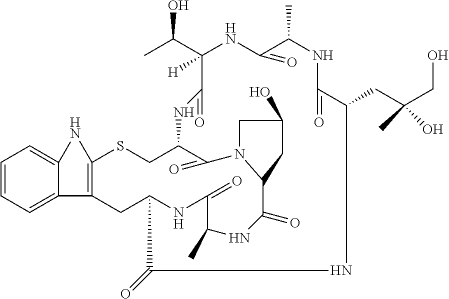 |
| Jaspamide Formula 12 | 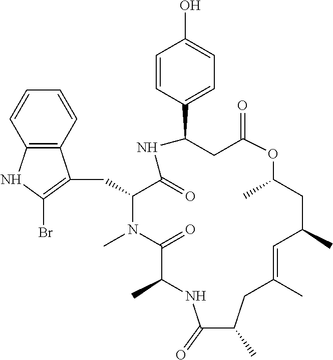 [SMILES: C/C1=C\[C@H](C)C[C@H](C)OC(=O)C[C@H](c2ccc(O)cc2)NC(=O)[C@@H](Cc2c(Br)[nH]c3ccccc23)N(C)C(=O)[C@H](C)NC(=O)[C@@H](C)C1] |
| Dolastatin 11 Formula 13 | 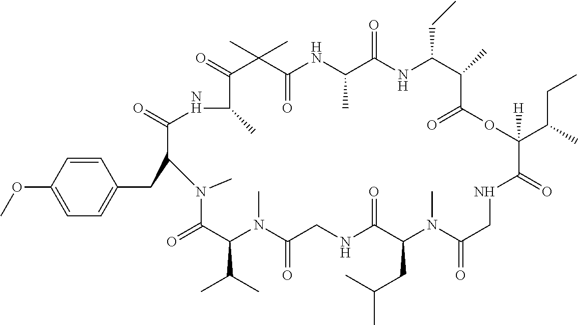 |
| Hectochlorin Formula 14 | 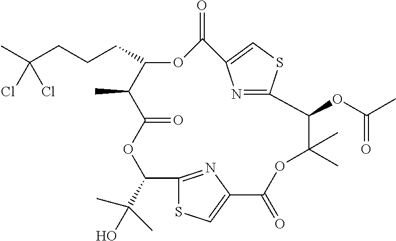 |
| Doliculide Formula 15 | 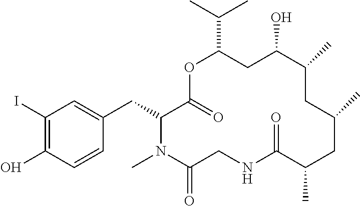 |
| Colchicine Formula 16 | 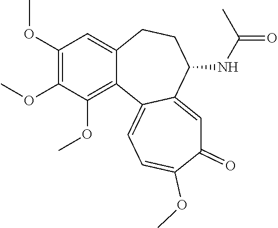 [SMILES: COc1cc2c(c(OC)c1OC)-c1ccc(OC)c(=O)cc1[C@@H](NC(C)=O)CC2] |
| Colcemid Formula 17 | 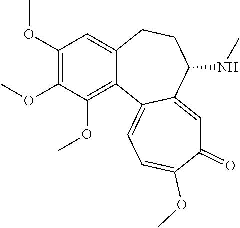 |
| Vinblastine Formula 18 | 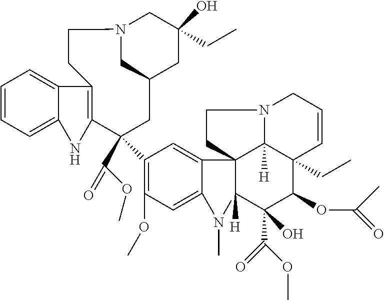 |
| Vincristine Formula 19 | 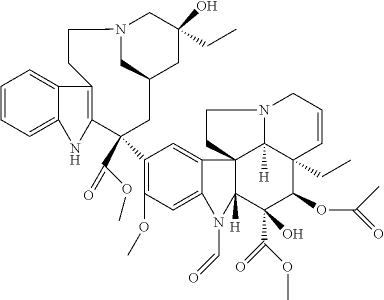 |
| Nocodazole Formula 20 | 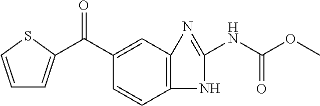 [SMILES: COC(=O)Nc1nc2cc(C(=O)c3cccs3)ccc2[nH]1] |
| Myoseverin Formula 21 | 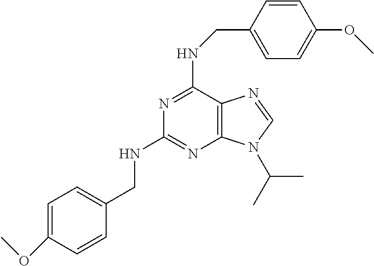 |
| Taxol (paditaxel) Formula 22 | 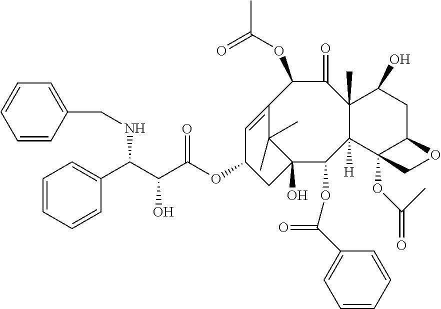 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NCc4ccccc4)c4ccccc4)C=C1C3(C)C)[C@]1(OC(C)=O)CO[C@@H]1C[C@@H]2O] |
| Epothilone B Formula 23 | 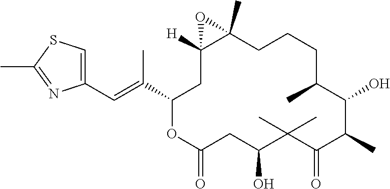 [SMILES: C/C(=C\c1csc(C)n1)[C@@H]1C[C@@H]2O[C@]2(C)CCC[C@H](C)[C@H](O)[C@@H](C)C(=O)C(C)(C)[C@@H](O)CC(=O)O1] |
| Eleutherobin Formula 24 | 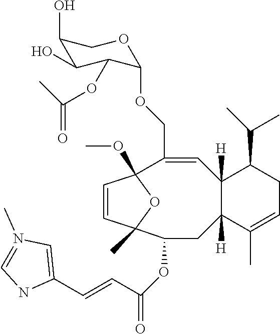 |
| Discodermolide Formula 25 | 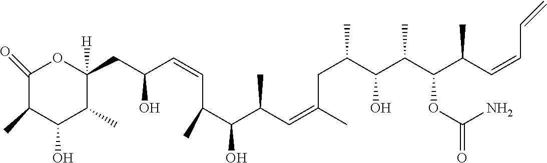 [SMILES: C=C/C=C\[C@H](C)[C@H](OC(N)=O)[C@@H](C)[C@H](O)[C@@H](C)C/C(C)=C\[C@H](C)[C@@H](O)[C@@H](C)/C=C\[C@@H](O)C[C@@H]1OC(=O)[C@H](C)[C@@H](O)[C@H]1C] |
| Laulimalide Formula 26 | 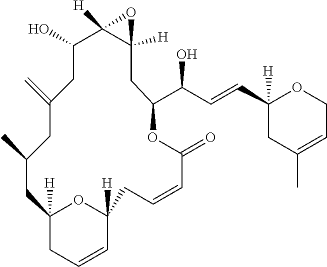 [SMILES: C=C1C[C@H](C)C[C@@H]2CC=C[C@@H](C/C=C\C(=O)O[C@H]([C@@H](O)/C=C/[C@@H]3CC(C)=CCO3)C[C@@H]3O[C@H]3[C@@H](O)C1)O2] |
| Migrastatin Formula 27 | 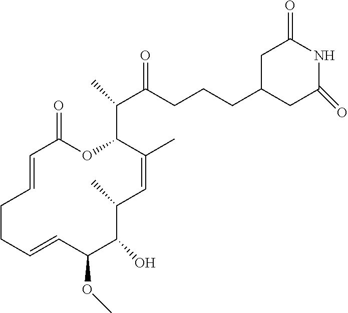 [SMILES: CO[C@H]1/C=C/CC/C=C/C(=O)O[C@H]([C@H](C)C(=O)CCCC2CC(=O)NC(=O)C2)/C(C)=C\[C@@H](C)[C@@H]1O] |
| Motuporamine C Formula 28 | 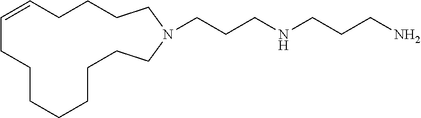 [SMILES: NCCCNCCCN1CCCC/C=C\CCCCCCCC1] |
| UIC-1005 Formula 29 | 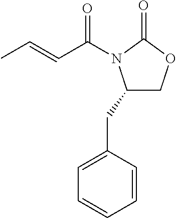 [SMILES: C/C=C/C(=O)N1C(=O)OC[C@@H]1Cc1ccccc1] |
| BDM Formula 30 | 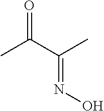 |
| BTS Formula 31 | 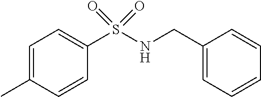 [SMILES: Cc1ccc(S(=O)(=O)NCc2ccccc2)cc1] |
| KTS5926 Formula 32 | 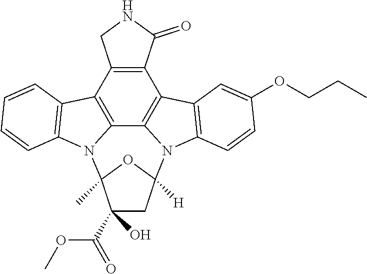 [SMILES: CCCOc1ccc2c(c1)c1c3c(c4c5ccccc5n5c4c1n2[C@H]1C[C@](O)(C(=O)OC)[C@]5(C)O1)CNC3=O] |
| ML-7 Formula 33 | 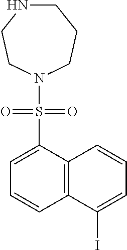 [SMILES: O=S(=O)(c1cccc2c(I)cccc12)N1CCCNCC1] |
| ML-9 Formula 34 | 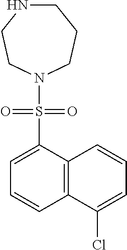 [SMILES: O=S(=O)(c1cccc2c(Cl)cccc12)N1CCCNCC1] |
| Y-27632 Formula 35 | 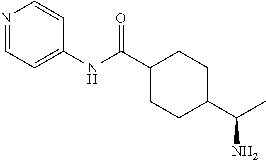 [SMILES: C[C@@H](N)C1CCC(C(=O)Nc2ccncc2)CC1] |
| HA1077 Formula 36 | 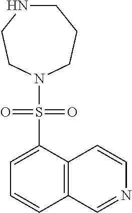 [SMILES: O=S(=O)(c1cccc2cnccc12)N1CCCNCC1] |
| H1152 Formula 37 | 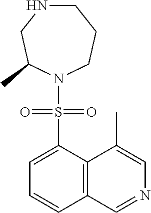 [SMILES: Cc1cncc2cccc(S(=O)(=O)N3CCCNC[C@@H]3C)c12] |

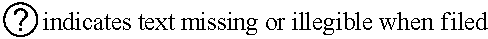

It should be noted that among the above described candidate modulators, the present EXAMPLES have already established utility of members of inhibitors of actin depolymerization, F-actin stabilizer and inhibitors of at least one of myosinIIA phosphorylation and activity and inhibitors of upstream signaling molecules, specifically, JAS, Y-27632 and CytD or any combinations thereof, for modulating ARF and lymphocyte activation, and NK activation forming NKIS in particular.

Thus, in some embodiments, the invention provides the use of JAS, specifically as denoted by Formula 12, that that modulate ARF for lymphocyte activation, and in some specific embodiments, NK activation forming NKIS in particular.

In yet some further embodiments, the invention provides the use of Y-27632, specifically as denoted by Formula 35, that modulate ARF for lymphocyte activation, and in some specific embodiments for activation of NK cells forming NKIS in particular.

In some other embodiments, the invention provides the use of CytD, specifically as denoted by Formula 2, that that modulate ARF for lymphocyte activation, and NK activation forming NKIS in particular.

It is thus contemplated that in specific embodiments the modulators of the invention that are ARF inhibitors are at least one of inhibitors of actin depolymerization, F-actin stabilizers, and at least one of inhibitors of myosinIIA phosphorylation and activity.

It should be appreciated that the invention encompasses the use of any of the above-indicated modulators of the present disclosure as well as Table 1, any functional derivatives thereof or any combinations thereof. It should be further understood, that the inhibitors disclosed by the invention serve as non-limiting examples, and moreover, it should be appreciated that the invention further encompass any further inhibitor that functions in modulation of the ARF as described above, as well as any small molecule, any peptide or any compound that may modulate the ARF or alternatively, may modulate the interaction between said beta-actin and SHP-1 as described by the invention.

In further specific and non-limiting embodiments, the modulators used by the invention according to the above may be any one of Jasplakinolide (JAS), Rho kinase inhibitor of myosin light chain (MLC) phosphorylation (Y-27632), and Cytochalasin D, any combination thereof or any vehicle, matrix, nano- or micro-particle comprising the same.

It should be appreciated that in some embodiments, the invention provides the use of any of the modulators disclosed herein, in modulating lymphocyte cell activation in a subject in need thereof.

In some further embodiments, the invention provides the use of any of the modulators disclosed herein, in a method of treating, preventing, inhibiting, reducing, eliminating, protecting or delaying the onset of an immune-related disorder in a subject in need thereof.

In more specific embodiments, such immune-related disorder may be at least one of a viral infection, a proliferative disorder, a graft versus host disease, an inflammatory disorder, an immune-cell mediated disorder and an autoimmune disorder.

It is another important aspect of the present invention to provide a composition comprising an effective amount of a modulator or any vehicle, matrix, nano- or micro-particle comprising the same, for use in a method for of hematopoietic cell activation. The modulators used by the compositions of the invention are characterized in that they modulate ARF in a cell. In some embodiments the composition may optionally further comprise at least one of pharmaceutically acceptable carrier/s, excipient/s, auxiliaries, and/or diluent/s. In some specific embodiments, the compositions of the invention may be used for modulating the activation of lymphocytes. In ore specific embodiments, the modulators comprised within the compositions used by the invention are characterized in that they modulate ARF in a lymphocyte cell forming an activating or inhibitory IS.

In some embodiments, the compositions of the invention may be used for modulating activation of lymphocytes such as at least one of an NK cell, a T cell and a B cell forming an inhibitory or activating IS.

In fact, use of the modulators of the invention in liposomal NPs has been already achieved and proven successful to control NK cell responsiveness (see EXAMPLE 9).

Thus in specific embodiments the compositions for use according to the invention, namely those comprising the relevant modulators as described above, are applicable to modulation of NK cells forming activating or inhibitory NKIS.

In further specific embodiments, the compositions for use according to the invention are applicable to modulation of NK cells forming inhibitory NKIS, wherein said modulator comprised within these compositions inhibits or disturbs ARF in NK cells.

In yet further specific embodiments, the compositions for use according to the invention, especially those inhibiting or disrupting ARF, lead to formation of complex comprising β-actin and at least one phosphatase, specifically, at least one PTP in said NK cell. In more specific embodiments, the compositions used by the invention may lead to formation of complex comprising β-actin and at least SH2 domain-containing phosphatase in said NK cell. In still further specific embodiments, the compositions for use according to the above lead to the formation of a specific complex between the SH2 domain-containing phosphatase SHP-1 and the β-actin, which thereby induces a change in the SHP-1 conformation and catalytic activity in NK cells.

In yet some further specific embodiments, the compositions for use according to the above lead to the formation of a specific complex between the SH2 domain-containing phosphatase SHP-2 and the β-actin, which thereby induces a change in the SHP-2 conformation and catalytic activity in NK cells.

Thus, it should be appreciated that the compositions of the invention may be used in methods for modulating the conformation and catalytic activity of SHP-1 and/or SHP-2 in a cell.

In other words, in some embodiments the general purpose for use of the compositions of the invention is to modulate, meaning activate or inhibit, cytotoxic activity lymphocytes or NK cells forming in IS. In specific embodiments, the compositions are intended to activate nascent NK cells forming inhibitory NKIS.

In numerous embodiments, the compositions for use according to the invention, those that are activating of NK cells as above, lead to increase in at least one of intracellular Ca²⁺ flux, and secretion or formation of cytolytic granules in these NK cells.

In yet some further embodiments, the invention provides compositions for use in methods for modulating the phosphorylation of VAV1. In yet some further specific embodiments, the invention provides the use of compositions comprising ARF inhibitors for increasing the phosphorylation of VAV 1 in a cell.

In yet some further embodiments, the invention provides compositions for use in methods for modulating the phosphorylation of PLCγ1/2. In yet some further specific embodiments, the invention provides the use of compositions comprising ARF inhibitors for increasing the phosphorylation of PLCγ1/2 in a cell.

Still further, the invention provides the use of compositions comprising ARF inhibitors for increasing killing of target cells by NK cells.

It should be appreciated that “increase”, as used herein in connection with phosphorylation of VAV1 or PLCγ1/2, or alternatively, killing of target cells by NK cells activated by the modulators of the invention, compositions and methods described herein after, relates to “elevation”, “augmentation” and “enhancement” as referring to the act of becoming progressively greater in size, amount, number, or intensity. Particularly, an increase of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, 300%, 400%, 500%, 600%, 700%, 800%, 900% or about 1000% increase.

As previous discussed, in specific embodiments the compositions used according to the invention, especially those acting as ARF inhibitors, can comprise at least one inhibitor of actin depolymerization or an F-actin stabilizer, and/or an inhibitor of myosinIIA phosphorylation and/or myosinIIA activity.

In further specific embodiments such compositions for use can comprise an ARF inhibitor that is any one of JAS, CytD and Y-27632, or any derivatives or combinations thereof.

It should be appreciated that the invention further encompasses the use of compositions comprising any of the modulators described herein or any derivatives, formulations or combinations thereof.

It should be noted that in all of the above numerous embodiments, the compositions for use in accordance with the invention may comprise one or more kind of the modulators of ARF and optionally one or more additional known therapeutic agents to achieve the desirable level and kind of control on lymphocyte activation.

Still further, in some embodiments, the composition for use according to the invention, may be particularly applicable for use in modulating lymphocyte cell activation in a subject in need thereof.

In yet some further embodiments, the invention provides the compositions of the invention for use in a method of treating, preventing, inhibiting, reducing, eliminating, protecting or delaying the onset of an immune-related disorder in a subject in need thereof.

In some embodiments, such immune-related disorder is at least one of a viral infection, a proliferative disorder, specifically, cancer, a graft versus host disease, an inflammatory disorder, an immune-cell mediated disorder and an autoimmune disorder.

In general terms, under compositions herein is meant predominantly pharmaceutical compositions, meaning that such compositions would comprise a therapeutically effective amount of at least one active agent, i.e. a modulator according to the invention, and optionally, at least one pharmaceutically acceptable carrier. The term ‘pharmaceutically acceptable’ means approved by a regulatory agency or listed in the U.S. Pharmacopeia or other generally recognized pharmacopeia for use in animals, and more particularly in humans.

The term ‘carrier’ denotes to a diluent, adjuvant, excipient, or vehicle with which the therapeutic is administered. Example of such pharmaceutical carriers are sterile liquids, such as water and oils, including those of petroleum, animal, vegetable or synthetic origin, such as peanut oil, soybean oil, mineral oil, sesame oil and the like. Suitable pharmaceutical excipients may include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A composition can further contain minor amounts of wetting or emulsifying agents, or pH buffering agents. The compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In specific embodiments, the compositions of the invention may be formulated in accordance with routine procedures as pharmaceutical compositions adapted for intravenous administration in humans. Where necessary, the composition may also include a solubilizing agent and a local anesthetic such as lidocaine to ease pain at the site of the injection. Where the composition is administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Combination Drugs

In numerous embodiments, the compositions of the present invention may be administered in a form of combination therapy, i.e. in combination with one or more additional therapeutic agents. Combination therapy may include administration of a single pharmaceutical dosage formulation comprising at least one composition of the invention and additional therapeutics agent(s); as well as administration of at least one composition of the invention and one or more additional agent(s) in its own separate pharmaceutical dosage formulation. Further, where separate dosage formulations are used, compositions of the invention and one or more additional agents can be administered concurrently or at separately staggered times, i.e. sequentially. Still further, said concurrent or separate administrations may be carried out by the same or different administration routes.

In specific embodiments, compositions of the invention are administered with one or more therapeutic agents specifically relevant to viral infection, cancer, a proliferative disorder, a graft versus host disease, an inflammatory disorder, an immune-cell mediated disorder and an autoimmune disorder.

More specifically in the present invention, it is contemplated that the other therapeutic agent may involve the administration or inclusion of at least one additional factor that may in some specific embodiments be selected from among EPO, G-CSF, M-GDF, SCF, GM-CSF, M-CSF, CSF-1, IL-1, IL-2, IL-3, IL-4, IL-5, IL-6, IL-7, IL-8, IL-9, IL-10, IL-11, IL-12, or other various interleukins, IGF-1, LIF, interferon (such as a, beta, gamma or consensus), neurotrophic factors (such as BDNF, NT-3, CTNF or noggin), other multi-potent growth factors (such as, to the extent these are demonstrated to be such multi-potent growth factors, flt-3/flk-2 ligand, stem cell proliferation factor, and totipotent stem cell factor), fibroblast growth factors (such as FGF), and analogs, fusion molecules, or other derivatives of the above.

Alternatively, treatment with the modulators of the invention, may precede or follow the other agent treatment by intervals ranging from minutes to weeks. In embodiments where the other therapeutic agent and the Modulators are administered separately, one would generally ensure that a significant period of time did not expire between the time of each delivery, such that the other agent and the Modulators would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In some specific and non-limiting embodiments, the modulators used by the invention may be applicable in combined treatment with G-CSF. Granulocyte-colony stimulating factor (G-CSF), also known as colony-stimulating factor 3 (CSF 3), is a glycoprotein that stimulates the bone marrow to produce granulocytes and stem cells and release them into the bloodstream. Functionally, it is a cytokine and hormone, a type of colony-stimulating factor, and is produced by a number of different tissues. The pharmaceutical analogs of naturally occurring G-CSF are called filgrastim and lenograstim. G-CSF also stimulates the survival, proliferation, differentiation, and function of neutrophil precursors and mature neutrophils.

In oncology and hematology, a recombinant form of G-CSF is used with certain cancer patients to accelerate recovery and reduce mortality from neutropenia after chemotherapy, allowing higher-intensity treatment regimens. G-CSF is also used to increase the number of hematopoietic stem cells in the blood of the donor before collection by leukapheresis for use in hematopoietic stem cell transplantation. G-CSF may also be given to the receiver in hematopoietic stem cell transplantation, to compensate for conditioning regimens.

Formulations Adapted for Various Administrations

The pharmaceutical compositions of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ, etc. More specifically, the compositions used in the methods and compositions of the invention, described herein after, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). It should be noted that any of the administration modes discussed herein, may be applicable for any of the methods of the invention as described in further aspects of the invention herein after.

Compositions and formulations for oral administration may include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable. Pharmaceutical formulations adapted for rectal administration may be presented as suppositories or enemas. Pharmaceutical formulations adapted for vaginal administration may be presented as pessaries, tampons, creams, gels, pastes, foams or spray formulations.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

The compositions of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis.

Formulations for ocular and aural administration may be formulated to be immediate and/or modified release. Modified release includes delayed, sustained, pulsed, controlled, targeted, and programmed release.

In specific embodiments, the unit dosage formulations are those containing a daily dose or sub-dose, as herein above recited, or an appropriate fraction thereof, of an active ingredient.

Of particular relevance are formulations of compositions of the invention adapted for use as a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as liposomes or polyelectrolyte multilayer capsules, incorporate nanoparticle (NP) actuators.

More specifically, Controlled drug delivery systems (DDS) have several advantages compared to the traditional forms of drugs. A drug is transported to the place of action, hence, its influence on vital tissues and undesirable side effects can be minimized Accumulation of therapeutic compounds in the target site increases and, consequently, the required doses of drugs are lower. This modern form of therapy is especially important when there is a discrepancy between the dose or the concentration of a drug and its therapeutic results or toxic effects. Cell-specific targeting can be accomplished by attaching drugs to specially designed carriers. Various nanostructures, including liposomes, polymers, dendrimers, silicon or carbon materials, and magnetic nanoparticles, have been tested as carriers in drug delivery systems. Polymeric nanoparticles are one technology being developed to enable clinically feasible oral delivery.

The term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid. Examples of lipids forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS). Examples of cationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N—(N′,N′-dimethylaminoethane) carbamoly] cholesterol (DC-Chol); and dimethyl-dioctadecylammonium (DDAB), N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS), or the neutral lipid dioleoylphosphatidyl ethanolamine (DOPE) derivatized with polylysine to form a cationic lipopolymer.

The lipids may be combined with other lipid compatible substances, such as, sterols, lipopolymers etc. A lipopolymer may be a lipid modified by inclusion in its polar headgroup a hydrophilic polymer. The polymer headgroup of a lipopolymer may be preferably water-soluble. In some embodiments, the hydrophilic polymer has a molecular weight equal or above 750 Da. There are numerous polymers which may be attached to lipids to form such lipopolymers, such as, without being limited thereto, polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers. The lipids derivatized into lipopolymers may be neutral, negatively charged, as well as positively charged. The most commonly used and commercially available lipids derivatized into lipopolymers are those based on phosphatidyl ethanolamine (PE), usually, distearoylphosphatidylethanolamine (DSPE).

In some embodiments, the structure forming lipids may be combined with other lipids, such as a sterol. Sterols and in particular cholesterol are known to have an effect on the properties of the lipid's organized structure (lipid assembly), and may be used for stabilization, for affecting surface charge, membrane fluidity.

In some embodiments, a sterol, e.g. cholesterol is employed in order to control fluidity of the lipid structure. The greater the ratio sterol:lipids (the structure forming lipids), the more rigid the lipid structure is.

Liposomes are often distinguished according to their number of lamellae and size. The liposomes employed in the context of the present disclosure may be multilamellar vesicles (MLVs), multivesicular vesicles (MVVs), small unilamellar vesicles (SUVs), large unilamellar vesicles (LUVs) or large multivesicular vesicles (LMVV).

It should be appreciated that the at least one modulators used by the invention may be associated with any of the nanostructures described above, specifically, any of the micellar formulations, liposomes, polymers, dendrimers, silicon or carbon materials, polymeric nanoparticles and magnetic nanoparticles disclosed herein above. The term “association” may be used interchangeably with the term “entrapped”, “attachment”, “linked”, “embedded”, “absorbed” and the like, and contemplates any manner by which the at least one modulators of the invention is held. This may include for example, physical or chemical attachment to the carrier. Chemical attachment may be via a linker, such as polyethylene glycol. The association provides capturing of the at least one modulators of the invention by the nanostructure such that the release of the at least one modulators used by the invention may be controllable. Still further, it should be appreciated that in some embodiments, the nanostructure in accordance with the present disclosure may further comprise at least one targeting moiety on the surface. Such targeting moiety may facilitate targeting the modulators-nanostructures of the invention into a particular target cell, target tissue, target organ or particular cellular organelle target. The transporting or targeting moiety may be attached directly or indirectly via any linker, and may comprise affinity molecules, for example, antibodies that specifically recognize target antigen on specific hematopoietic cells.

In fact, use of the modulators of the invention in liposomal NPs has been already achieved and proven successful to control NK cell responsiveness (see EXAMPLE 9).

Thus, in some embodiments, the invention provides nanoparticles, specifically, liposomes that comprise any of the ARF modulators described by the invention (specificaly those disclosed in the description and in Table 1), or any composition or combinations thereof. In some embodiments, such liposomes or nanoparticles may comprise a targeting moiety. In some embodiments such targeting moiety may facilitate targeting the liposome to a desired target cell. In more specific embodiments, the modulators of the invention may be comprised within liposomes targeted at NK cells. In some embodiments such liposomes may be coated with HA (CD44 ligand). In yet some further embodiments, the liposomes may be coated with anti-NKp46 antibody. In further embodiments, the liposomes may be coated with both, HA and anti-NKp46 antibody. Still further, in some other specific embodiments the modulators of the invention may be comprised within LFA1 coated NPs.

Still further, the nanoparticles provided by the invention may be in some particular embodiments multilamellar liposomes, composed of phosphatidylcholine (PC), dipalmitoyl phosphatidyl-ethanolamine (DPPE), and cholesterol (Chol). In more specific embodiments, such nanoparticles may be presented at molar ratios of 3:1:1 (PC:DPPE:Chol) as described in EXAMPLE 9 and FIG. 15.

Thus, in some specific embodiments, the invention provides nanoparticles composed of PC:DPPE:Chol, that may be coated with at least one of HA, anti-NKp46 antibody and LFA1, that comprise an effective amount of JAS. In some further specific embodiments, the invention provides nanoparticles composed of PC:DPPE:Chol, that may be coated with at least one of HA and anti-NKp46 antibody, that comprise an effective amount of Y-27. In yet some further specific embodiments, the invention provides nanoparticles composed of PC:DPPE:Chol, that may be coated with at least one of HA and anti-NKp46 antibody, that comprise an effective amount of CytD.

In providing this kind of modulators and compositions related thereto, it is further important aspect of the present invention to provide a method for modulating hematopoietic cell activation. More specifically, the method of the invention may comprise contacting the cell with a modulatory effective amount of a modulator or any vehicle, matrix, nano- or micro-particle, or composition comprising the same. In certain embodiments, the modulator may be characterized in that it modulates ARF in a cell This method in its various embodiments can be implemented in vivo and in vitro for research purposes as well as for in various therapeutic purposes in vertebrate, mammalian, including human cells and organisms. In yet some further specific embodiments, the methods of the invention may be used for modulating the activation of lymphocytes. In more specific embodiments, the modulators used by the methods of the invention are characterized in that they modulate ARF in a lymphocyte cell forming an activating or inhibitory IS.

The term “contacting” means to bring, put, incubate or mix together. As such, a first item is contacted with a second item when the two items are brought or put together, e.g., by touching them to each other or combining them. In the context of the present invention, the term “contacting” includes all measures or steps, which allow interaction between the modulators of the invention and the lymphocyte cells to be modulated.

In some embodiments, the methods according to the invention may be applied to lymphocyte cells that may be at least one of an NK cell, a T cell and a B cell forming an inhibitory or activating IS.

In specific embodiments, the methods according to the invention may be applied to lymphocyte cells that are NK cells forming an inhibitory or activating NKIS to achieve activation of nascent NK cells.

In further specific embodiments, the methods according to the invention, specifically those directed at activation of NK cells in inhibitory NKIS, use a type of modulators that inhibit or disturb ARF in NK cells.

In still further specific embodiments, the methods according to the invention using the type of modulation as above, namely inhibition or disruption of ARF in NK cells, lead to at least one of formation of a complex comprising β-actin and at least one phosphatase, specifically, at least one PTP in NK cells, change in the conformation of at least one PTP and change in the catalytic activity of said PTP. In more specific embodiments, the method of the invention may result in formation of a complex between beta-actin and SH2 domain-containing phosphatase in NK cells. Still further, in some embodiments, the invention provides methods using ARF modulators in the modulation of the conformation and/or catalytic activity of at least one of SHP-1 and SHP-2 in a cell.

In still further specific embodiments, such methods lead to formation of a complex between the SH2 domain containing phosphatase SHP-1 and the β-actin, namely the β-actin:SHP-1 complex, which in turn induce a change in the SHP-1 conformation and catalytic activity in the NK cells.

In numerous embodiments, the methods of the invention, specifically those directed to activation of NK cells, result in an increase in at least one of intracellular Ca²⁺ flux, and secretion of cytolytic granules in the NK cells.

In specific embodiments, the methods of the invention, specifically those using ARF inhibitors, apply at least one of inhibitors of actin depolymerization, F-actin stabilizers, and/or inhibitors of at least one of myosinIIA phosphorylation and activity.

In further specific embodiments, such methods apply any one of JAS, CytD and Y-27632 or any derivatives thereof, which were demonstrated as effective ARF inhibitors in lymphocytes and NK cells in particular.

For therapeutic applications, the methods according to the invention may be used for modulating lymphocyte cell activation in a subject in need thereof. In some specific embodiments, such methods may comprise administering to the subject a modulatory effective amount of the above disclosed modulators, specifically, any modulator that modulates at least one of actin and myosin ARF in a lymphocyte cell forming an activating or inhibitory IS, or of any vehicle, matrix, nano- or micro-particle, or composition comprising the same.

In yet some further embodiments, modulation of lymphocyte cell activation using the compositions and methods of the invention may be relevant for a mammalian subject suffering of an immune-related disorder. In more specific embodiments, such immune-related disorder may be at least one of a viral infection, cancer or any other a proliferative disorder, a graft versus host disease, an inflammatory disorder, an immune-cell mediated disorder and an autoimmune disorder.

An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., viral infections), metabolic disorders and a proliferative disorder, specifically, cancer.

In some specific embodiments wherein the immune-related disorder or condition may be a primary or a secondary immunodeficiency. It should be understood that any of the immune-related disorders described herein after in connection with other aspects of the invention are also applicable or the present aspect as well.

A further aspect of the invention relates to a method for treating, preventing, inhibiting, reducing, eliminating, protecting or delaying the onset of an immune-related disorder in a subject in need thereof. In some embodiments, the method comprising administering to the treated subject a therapeutically effective amount of at least one modulator that modulates at least one of actin and myosin ARF in a cell, or of any vehicle, matrix, nano- or micro-particle, or composition comprising the same. In more specific embodiments, the modulators used by the method of the invention may modulate at least one of actin and myosin ARF in a lymphocyte cell forming an activating or inhibitory IS.

In some embodiments, the lymphocyte cell may be at least one of an NK cell, a T cell and a B cell forming an inhibitory or activating IS.

In more specific embodiments, the lymphocyte cell may be a NK cell forming an activating or inhibitory NKIS. Still further, in some embodiments, the methods of the invention encompasses the use of modulators that activate NK cells in an inhibitory NKIS. More specifically, such modulator/s disturb/s and/or inhibit/s ARF in the NK cell.

In further embodiments, disruption or inhibition of ARF by the modulators used by the methods of the invention results in formation of a complex comprising β-actin and at least one PTP in said NK cell. In more specific embodiments, such PTP may be SHP, for example, SHP-1 and/or SHP-2, more specifically, SHP-1. In further embodiments, the β-actin:SHP-1 complex induces a change in the SHP-1 conformation and catalytic activity in the NK cell.

In further embodiments, activation of NK cells by the modulators used by the methods of the invention may result in an increase in at least one of intracellular Ca²⁺ flux and secretion of cytolytic granules in said NK cell.

In yet some further embodiments, the invention provides methods for modulating the phosphorylation of VAV1. In yet some further specific embodiments, the invention provides methods using ARF inhibitors for increasing the phosphorylation of VAV1 in a cell.

Still further in some further embodiments, the invention provides methods for modulating the phosphorylation of PLCγ1/2. In yet some further specific embodiments, the invention provides methods using ARF inhibitors for increasing the phosphorylation of PLCγ1/2 in a cell.

The invention provides in some embodiments thereof methods using ARF inhibitors for increasing killing of target cells by NK cells.

In further embodiments, the modulators used by the invention may be ARF inhibitors. Theses inhibitors that may be at least one of an inhibitor of actin depolymerization, an F-actin stabilizer, and an inhibitor of at least one of myosinIIA phosphorylation and activity.

In some specific and non-limiting examples, the ARF inhibitor used by the method of the invention may be any one of JAS, Y-27632, CytD, or any derivatives or any combinations thereof or any nanoparticles comprising the same. In some embodiments, the methods of the invention may use any of the liposomes or compositions thereof as described by the invention herein before.

It should be appreciated that any of the ARF modulators discussed herein, specifically in Table 1, may be used for any of the compositions and methods of the invention.

As noted above, the methods of the invention may be relevant for treating any immune-related disorder, for example, a viral infection, cancer or any other proliferative disorder, a graft versus host disease, an inflammatory disorder, an immune-cell mediated disorder and an autoimmune disorder. The term an ‘immune-related disorder’, as meant herein, encompasses a range of dysfunctions of the innate and adaptive immune systems. In more specific terms, immune-related disorder can be characterized, for example, (1) by the component(s) of the immune system; (2) by whether the immune system is overactive or underactive; (3) by whether the condition is congenital or acquired.

In some specific embodiments, the methods of the invention may be used for treating cancer or any other proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for treatment of a patient suffering from any one of non-solid and solid tumors.

Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma

In some embodiments, the method of the invention may be used to treat a proliferative disorder, cancer, tumor and malignancy by activating/enhancing antitumor immunity. The term “antitumor immunity” refers to innate and adaptive immune responses which may lead to tumor control.

The immune system can be activated by tumor antigens and, once primed, can elicit an antitumor response. Activated tumor specific cytotoxic T lymphocytes (CTLs) can seek out and destroy metastatic tumor cells and reduce tumor lesions. Natural Killer (NK) cells are a front-line defense against drug-resistant tumors and can provide tumoricidal activity to enhance tumor immune surveillance Cytokines like IFN-γ or TNF play a crucial role in creating an immunogenic microenvironment and therefore are key players in the fight against metastatic cancer. Critical aspects in the tumor-immune system interface include the processing and presentation of released antigens by antigen-presenting cells (APCs), interaction with T lymphocytes, subsequent immune/T-cell activation, trafficking of antigen-specific effector cells, and, ultimately, the engagement of the target tumor cell by the activated effector T cell.

Nevertheless, although often successful in preventing tumor outgrowth, this “cancer-immunity cycle” can be disrupted by artifices involved in immune escape and development of tolerance, culminating with the evasion and proliferation of malignant cells. Furthermore, the tumor microenvironment induces suppression and reduced activity of NK and T cells, through the secretion of inhibitory factors suppressing the anti-tumor response, a phenomena known as exhaustion. Using modulators of ARF, the invention provides methods and compositions for activation of lymphocytes, specifically, NK cells, T cells and/or B cells, for enhancing anti-tumor immunity

Still further, of particular relevance are patients' populations diagnosed with one of autoimmune disorders, also referred to as disorders of immune tolerance, when the immune system fails to properly distinguish between self and non-self antigens. It has been well established that T cells lymphocytes, and the NK cells in particular, play a pivotal role in the control of immune tolerance under normal conditions, and in T- and B-cell mediated human autoimmune disorders. The NK cells have been further implicated in rheumatoid arthritis, systemic lupus erythematosus, and in multiple sclerosis.

Thus, according to some embodiments, the method of the invention may be used for the treatment of a patient suffering from any autoimmune disorder. In some specific embodiments, the methods of the invention may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behçet's syndrome, Indeterminate colitis, rheumatoid arthritis, systemic lupus erythematosus (SLE), Eaton-Lambert syndrome, Goodpasture's syndrome, Greave's disease, Guillain-Barr syndrome, autoimmune hemolytic anemia (AIHA), Idiopathic thrombocytopenic purpura (ITP), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

Of particular interest to the present context is a condition denoted Graft versus Host Disease (GvHD) that may occur after an allogeneic transplant, wherein the donated transplant cells view the recipient's body as foreign. GvHD is a possible complication of high dose cancer treatment. It also happens after an allogeneic bone marrow or stem cell transplant that use very high doses of chemotherapy, sometimes with radiotherapy. The term ‘GvHD’ as meant herein encompasses all known form of GvHD, namely the acute GvHD (aGvHD), the chronic GvHD (cGvHD), and the late acute GVHD and overlap syndrome (with features of both aGvHD and cGvHD).

More specifically, the pathophysiology of aGvHD has been tightly linked to the activity and maturation of the donor T cells and NK cells that are transferred along with the marrow graft, i.e. cells that are directly responsible for recognition of antigenic differences on antigen-presenting cells of the host. Once activated, donor anti-host-specific T cells can mediate tissue destruction. GvHD continues to be a major life-threatening complication after allogeneic bone marrow transplantation.

In other words, use of the modulators according to the present invention is particularly relevant in patients diagnosed with one of the types of GvHD. Such patients may be recognized by specific manifestation of symptoms. In the classical sense, a GvHD is characterized by selective damage to the liver, skin (rash), mucosa, and the gastrointestinal tract. Other types of GvHD may further involve the hematopoietic system, e.g., the bone marrow and thymus, and the lungs in the form of immune-mediated pneumonitis. Differential diagnosis of GvHD is further based on specific biomarkers.

In specific embodiments, the modulator according to the present invention are applicable to patients that are at risk of developing GvHD. For example, recipients who have received peripheral blood stem cells/bone marrow from an HLA mismatched related donor (or from an HLA matched unrelated donor) have an increased risk of developing a GvHD.

In other words, it is meant that the compositions and methods of the present invention can be applied to prevent the development of aGvHD.

In yet other embodiments, the methods of the invention may be also applicable for treating a subject suffering from an infectious disease. More specifically, such infectious disease may be any one of viral diseases, protozoan diseases, bacterial diseases, parasitic diseases, fungal diseases and mycoplasma diseases.

It should be appreciated that an infectious disease as used herein also encompasses any infectious disease caused by a pathogenic agent. Pathogenic agents include viruses, prokaryotic microorganisms, lower eukaryotic microorganisms, complex eukaryotic organisms, fungi, prions, parasites, yeasts, toxins and venoms.

A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, hepatitis A, poliovirus, rubella virus, hepatitis C, arboviruses, rabies virus, influenza viruses A and B, measles virus, mumps virus, HIV, HTLV I and II.

The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

The term “parasite” includes, but not limited to, infections caused by somatic tapeworms, blood flukes, tissue roundworms, ameba, and Plasmodium, Trypanosoma, Leishmania, and Toxoplasma species.

Still further, in certain embodiments, the methods and compositions of the invention may be applicable for treating disorders associated with immunodeficiency ‘Immunodeficiency’, primary or secondary, meaning inherited or acquired, respectively. The term ‘immunodeficiency’ is intended to convey a state of an organism, wherein the immune system's ability for immuno-surveillance of infectious disease or cancer is compromised or entirely absent.

According to the International Union of Immunological Societies, more than 150 primary immunodeficiency diseases (PIDs) have been characterized, and the number of acquired (or secondary) immuno-deficiencies exceeds the number of PIDs. PIDs are those caused by inherited genetic mutations. Secondary immuno-deficiencies are caused by various conditions, aging or agents such as viruses or immune suppressing drugs. A number of notable examples of PIDs include Severe combined immunodeficiency (SCID), DiGeorge syndrome, Hyperimmunoglobulin E syndrome (also known as Job's Syndrome), Common variable immunodeficiency (CVID): B-cell levels are normal in circulation but with decreased production of IgG throughout the years, so it is the only primary immune disorder that presents onset in the late teens. Chronic granulomatous disease (CGD): a deficiency in NADPH oxidase enzyme, which causes failure to generate oxygen radicals. Classical recurrent infection from catalase positive bacteria and fungi. Wiskott-Aldrich syndrome (WAS); autoimmune lymphoproliferative syndrome (ALPS); Hyper IgM syndrome: X-linked disorder that causes a deficiency in the production of CD40 ligand on activated T-cells. This increases the production and release of IgM into circulation. The B-cell and T-cell numbers are within normal limits. Increased susceptibility to extracellular bacteria and opportunistic infections. Leukocyte adhesion deficiency (LAD); NF-κB Essential Modifier (NEMO) Mutations; Selective immunoglobulin A deficiency: the most common defect of the humoral immunity, characterized by a deficiency of IgA. Produces repeating sino-pulmonary and gastrointestinal infections. X-linked agammaglobulinemia (XLA; also known as Bruton type agammaglobulinemia): characterized by a deficiency in tyrosine kinase enzyme that blocks B-cell maturation in the bone marrow. No B-cells are produced to circulation and thus, there are no immunoglobulin classes, although there tends to be a normal cell-mediated immunity. X-linked lymphoproliferative disease (XLP); and Ataxia-telangiectasia.

Thus patients' populations diagnosed with one of PIDs can particularly benefit from methods and compositions of modulators according to the present invention.

With respect to secondary immunodeficiencies, those can be manifested in both the young and the elderly. Under normal conditions immune responses are beginning to decline at around 50 years of age, what is called immunosenescence. The term ‘immunosenescence’ refers to the gradual deterioration of the immune system brought on by natural age advancement. It involves both the host's capacity to respond to infections and the development of long-term immune memory. Additional common causes of secondary immunodeficiency include severe burns, malnutrition, certain types of cancer, and chemotherapy in cancer patients.

More specifically, in developed countries, obesity, alcoholism, and drug use are common causes of poor immune function. However, malnutrition is the most common cause of immunodeficiency in developing countries. Diets lacking sufficient protein are associated with impaired cell-mediated immunity, complement activity, phagocyte function, IgA antibody concentrations, and cytokine production. Additionally, the loss of the thymus at an early age through surgical removal, for example, results in severe immunodeficiency and high susceptibility to infections.

Of particular relevance to the present context are cellular immunodeficiencies associated with cancer and certain viral pathogens. A cellular immunodeficiency refers to a deficiency the count or function of T lymphocytes, which are the main type of cells responsible for the cellular adaptive immune response in attacking viruses, cancer cells and other parasites. Extensive research has reasonably well established the role of immunodeficiency in cancers of the head and neck, lung, esophagus and breast. Among virally induced immunodeficiencies, the most notable example is AIDS (Acquired Immunodeficiency Syndrome) cause by HIV infection. The role of HIV as a direct cause of cellular immunodeficiency, particularly the deficiency of the CD4+T helper lymphocyte population, has been well established. Additional examples of viral- or pathogen-induced immunodeficiencies include, although not limited to chickenpox, cytomegalovirus, German measles, measles, tuberculosis, infectious mononucleosis (Epstein-Barr virus), chronic hepatitis, lupus, and bacterial and fungal infections. One of the most recent examples is virus-induced Severe Acute Respiratory Syndrome (SARS). These and additional examples of disorders related to cellular immunodeficiency may include Aplastic anemia, Leukemia, Multiple myeloma, Sickle cell disease, chromosomal disorders such as Down syndrome, infectious diseases caused by pathogens such as Cytomegalovirus, Epstein-Barr virus, Human immunodeficiency virus (HIV), Measles and certain bacterial infections. Chronic kidney disease, Nephrotic syndrome, Hepatitis, Liver failure and other conditions caused by Malnutrition, alcoholism and burns.

Thus patients' populations diagnosed with one of the secondary immunodeficiencies, and particularly one of the cellular immunodeficiencies as above, can particularly benefit from methods and compositions of modulators according to the present invention. Differential diagnosis of such immunodeficient patients is routinely performed in various clinical settings.

Additional secondary immunodeficiencies may result following bone marrow (BM) transplantation, gene therapy or adaptive cell transfer.

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin). Performance of this medical procedure usually requires the destruction of the recipient's immune system using radiation or chemotherapy before the transplantation. To limit the risks of transplanted stem cell rejection or of severe graft-versus-host disease in allogeneic HSCT, the donor should preferably have the same human leukocyte antigens (HLA) as the recipient. In the case of a bone marrow transplant, the HSC are removed from a large bone of the donor, typically the pelvis, through a large needle that reaches the center of the bone. Peripheral blood stem cells are now the most common source of stem cells for HSCT. They are collected from the blood through a process known as apheresis. The donor's blood is withdrawn through a sterile needle in one arm and passed through a machine that removes white blood cells. The red blood cells are returned to the donor. The peripheral stem cell yield is boosted with daily subcutaneous injections of Granulocyte-colony stimulating factor (G-CSF), serving to mobilize stem cells from the donor's bone marrow into the peripheral circulation. It should be noted that amniotic fluid as well as umbilical cord blood may be also used as a source of stem cells for HSCT.

Gene therapy is the therapeutic delivery of nucleic acid polymers into a patient's cells as a drug to treat disease. Gene therapy is a way to fix a genetic problem at its source. The polymers are either translated into proteins, interfere with target gene expression, or possibly correct genetic mutations. The most common form uses DNA that encodes a functional, therapeutic gene to replace a mutated gene. The polymer molecule is packaged within a “vector”, which carries the molecule inside cells.

Adaptive cell transfer (ACT) is the transfer of cells into a patient. The cells may have originated from the patient or from another individual. The cells are most commonly derived from the immune system, with the goal of improving immune functionality and characteristics. In cancer immunotherapy, T cells are extracted from the patient, genetically modified and cultured in vitro and returned to the same patient.

Of further relevance are patients' populations diagnosed with stress-induced immune-related diseases. Many studies have shown that exposure to physical or psychological stress can affect disease outcomes in immune-related disorders such as viral and bacterial infections, contact dermatitis and allergy. Stress, being it acute and short, or chronic and persistent have been shown to influence and modify various components of the immune system, in particular stress has be related to leukocytosis, increased NK cell cytotoxicity and reduced proliferative response to mitogens.

Of further relevance are patients' populations diagnosed with one of hypersensitivities of an immune response or allergies. Hypersensitivities are divided into four classes (Type I-IV) based on the mechanisms involved and the time course of the hypersensitive reaction. Type I is an immediate or anaphylactic reaction, often associated with allergy; it is mediated by IgE antibodies that trigger degranulation of mast cells and basophils. Type II (also called antibody-dependent or cytotoxic) occurs when antibodies bind to antigens on the patient's own cells, marking them for destruction; it is mediated by IgG and IgM antibodies. Type III and Type IV (also known as cell-mediated or delayed type) are mediated by T cells, monocytes, and macrophages; Type IV reactions are involved in many autoimmune and infectious diseases. A partial list including the most common allergies includes but not limited to Seasonal allergy, Mastocytosis, Perennial allergy, Anaphylaxis, Food allergy, Allergic rhinitis and Atopic dermatitis.

Of further relevance are patients diagnosed with splenomegaly (enlargement of spleen) or hypersplenism. Splenomegaly of between 11-20 cm greater than 20 cm in the size of spleen has been associated with hemolytic anemias, and other diseases involving abnormal red blood cells being destroyed in the spleen, as well as with other disorders, including congestion due to portal hypertension, and infiltration by leukemias and lymphomas.

In further specific embodiments, compositions and methods of the present invention can be applied to prevent an immunodeficiency and/or GvHD in immunocomprimised cancer patients, being it a result of cancer itself (as mentioned above) or an adverse effect of high doses of chemotherapy or radiotherapy (which may induce burns).

A number of human diseases were specifically related to NK cell deficiency and deficient NKIS. Those include certain PIDs characterized by genetic aberrations that impair NK cells function. Several of these diseases induce a specific blockade in the stages leading to the formation of a functional lytic synapse. Most of these diseases can result in haemophagocytic lymphohistiocytosis (HLH), i.e. an inappropriately robust immune response to infection (typically with herpesviruses), which results in a persistent systemic inflammatory syndrome. This leads to the physiological symptoms of septic shock, but is also associated with the pathological finding of haematophagocytosis (the ingestion of red blood cells by phagocytes). The NK cells are most relevant to the HLH phenotype, given their localization to marginal zones in lymphoid organs after viral infection, their innate function early in the course of infection and their inherent ability to eliminate hyperactivated macrophages.

It is thus meant that the modulators according to the present invention are particularly applicable to patients diagnosed with one of the disorders related to NK cell or NKIS deficiency, or abnormal NK lytic granule trafficking. Notable examples of disorders belonging to this group are detailed below.
Leukocyte adhesion deficiency type I (LAD-I) results from a defect in the CD18 (β-integrin) component of leukocyte integrin heterodimers. Thus, LAD-I leukocytes do not appropriately adhere to inflamed or activated cells and cannot localize effectively to tissues and sites of inflammation. This leads to increased numbers of leukocytes in the blood and susceptibility to infectious diseases. Because early steps in NK-cell synapse formation—adhesion and activation signaling depend on integrins, NK cells from patients with LAD-I do not adhere to their target cells, resulting in defective cytotoxicity. LAD-I is also distinguished from other diseases discussed here because it does not lead to HLH.
Wiskott-Aldrich syndrome (WAS) results from a hematopoietic-cell-specific defect in actin reorganization and cell signaling due to WASP deficiency. Patients lacking WASP expression or expressing abnormal WASP have NK cells with decreased cytolytic capacity. Clinically, patients with WAS are susceptible to herpesviruse and can develop HLH, thereby demonstrating the functional relevance of WASP deficiency for the NK-cell lytic synapse. Formation of the lytic synapse is abnormal in NK cells from WAS patients and includes decreased F-actin accumulation and adhesion-receptor clustering at the synapse.
Chediak-Higashi syndrome (CHS) and Hermansky-Pudlak syndrome type II (HPS2) both affect the normal formation of lytic granules and lead to the presence of “giant” lytic granules. Both are also associated with albinism, which is caused by aberrant functioning of melanocytes, which pigment skin via secretion of melanosomes (an equivalent of lytic granules). CHS and HPS2 are similar in that they represent a failure in generation of the NK-cell lytic synapse at the end of the effector stages, as the abnormal lytic granules will not migrate along the microtubules to the MTOC.
Familial erythrophagocytic lymphohistiocytosis (FHL) types 3 and 4 are similar to CHS and HPS2, but are not associated with albinism demonstrating that the affected genes are not essential in melanocytes. FHL3 is caused by mutation in the UNC13D gene, which encodes MUNC13-4. FHL4 is caused by mutations in the STX11 gene, which encodes syntaxin-11.

As indicated above, the in some further embodiments, the modulators used by the invention may be applicable in boosting the immune response of a subject suffering from a secondary immunosuppression caused by chemotherapy, specifically, treatment with a chemotherapeutic agent.

“chemotherapeutic agent” or “chemotherapeutic drug” (also termed chemotherapy) as used herein refers to a drug treatment intended for eliminating or destructing (killing) cancer cells or cells of any other proliferative disorder. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Various different types of chemotherapeutic drugs are available. A chemotherapeutic drug may be used alone or in combination with another chemotherapeutic drug or with other forms of cancer therapy, such as a biological drug, radiation therapy or surgery.

Certain chemotherapy agents have also been used in the treatment of conditions other than cancer, including ankylosing spondylitis, multiple sclerosis, hemangiomas, Crohn's disease, psoriasis, psoriatic arthritis, rheumatoid arthritis, lupus and scleroderma.

Chemotherapeutic drugs affect cell division or DNA synthesis and function and can be generally classified into groups, based on their structure or biological function. The present invention generally pertains to chemotherapeutic agents that are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-CoA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents.

However, several chemotherapeutic drugs may be classified as relating to more than a single group. It is noteworthy that some agents, including monoclonal antibodies and tyrosine kinase inhibitors, which are sometimes referred to as “chemotherapy”, do not directly interfere with DNA synthesis or cell division but rather function by targeting specific components that differ between cancer cells and normal cells and are generally referred to as “targeted therapies”, “biological therapy” or “immunotherapeutic agent” as detailed below.

More specifically, as their name implies, alkylating agents function by alkylating many nucleophilic functional groups under conditions present in cells. Examples of chemotherapeutic agents that are considered as alkylating agents are cisplatin and carboplatin, as well as oxaliplatin. Alkylating agents impair cell function by forming covalent bonds with amino, carboxyl, sulfhydryl, and phosphate groups in various biologically-significant molecules. Examples of agents which function by chemically modifying DNA are mechlorethamine, cyclophosphamide, chlorambucil and ifosfamide. An additional agent acting as a cell cycle non-specific alkylating antineoplastic agent is the alkyl sulfonate agent busulfan (also known as Busulfex).

In some particular embodiments, the immune-suppressive condition may be caused by treatment with oxaliplatin. More specifically, Oxaliplatin is a platinum-based chemotherapy drug in the same family as cisplatin and carboplatin. It is typically administered in combination with fluorouracil and leucovorin in a combination known as Folfox for the treatment of colorectal cancer. Compared to cisplatin the two amine groups are replaced by cyclohexyldiamine for improved antitumour activity. The chlorine ligands are replaced by the oxalato bidentate derived from oxalic acid in order to improve water solubility. Oxaliplatin is marketed by Sanofi-Aventis under the trademark Eloxatin®.

Still Further, anti-metabolites (also termed purine and pyrimidine analogues) mimic the structure of purines or pyrimidines which are the building blocks of DNA and may thus be incorporated into DNA. The incorporation of anti-metabolites into DNA interferes with DNA syntheses, leading to abnormal cell development and division. Anti-metabolites also affect RNA synthesis. Examples of anti-metabolites include 5-fluorouracil (5-FU), azathioprine and mercaptopurine, fludarabine, cladribine (2-chlorodeoxyadenosine, 2-CdA), pentostatin (2′-deoxycoformycin, 2′-DCF), nelarabine, Floxuridine (FUDR), gemcitabine (Gemzar, a synthetic pyrimidine nucleoside) and Cytosine arabinoside (Cytarabine).

In yet some further embodiments, the Modulators of the invention may be applicable for boosting an immune-response in a subject treated with a chemotherapeutic agent that may be at least one Plant alkaloid and terpenoid. Plant alkaloids and terpenoids are agents derived from plants that block cell division by preventing microtubule function, thereby inhibiting the process of cell division (also known as “mitotic inhibitors” or “anti-mitotic agents”). Examples of alkaloids include the vinca alkaloids (e.g. vincristine, vinblastine, vinorelbine and vindesine) and terpenoids include, for example, taxanes (e.g. paclitaxel and docetaxel). Taxanes act by enhancing the stability of microtubules, preventing the separation of chromosomes during anaphase.

In further embodiments, the modulators used by the invention may be applicable for boosting an immune-response in a subject treated with chemotherapeutic agent that may be at least one Topoisomerase inhibitor. Topoisomerases are essential enzymes that maintain DNA topology (i.e. the overall three dimensional structure of DNA). Inhibition of type I or type II topoisomerases interferes with both transcription and replication of DNA by inhibiting proper DNA supercoiling. Type I topoisomerase inhibitors include camptothecins [e.g. irinotecan and topotecan (CPT11)] and examples of type II inhibitors include amsacrine, etoposide, etoposide phosphate, and teniposide.

Still further, Anthracyclines (or anthracycline antibiotics) are a class of drugs used in cancer chemotherapy that are derived from the streptomyces bacterium. These compounds are used to treat many cancers, including leukemias, lymphomas, breast, uterine, ovarian, and lung cancers. These agents include, inter alia, the drugs daunorubicin (also known as Daunomycin), and doxorubicin and many other related agents (e.g., Valrubicin and Idarubicin). For example, the anthracycline agent Idarubicin acts by interfering with the enzyme topoisomerase II.

In further embodiments, the modulators used by the invention may be applicable for boosting an immune-response in a subject treated with Doxorubicin. The chemotherapeutic agent Doxorubicin (also known by the trade name Adriamycin and by the name hydroxydaunorubicin) is an anthracycline antibiotic that is closely related to the natural product daunomycin, and like all anthracyclines, it works by intercalating DNA. The most serious adverse side effect of using this agent is the life-threatening heart damage. It is commonly used in the treatment of a wide range of cancers, including hematological malignancies, many types of carcinoma, and soft tissue sarcomas.

In certain embodiments, the modulators used by the invention may be applicable for boosting an immune-response in a subject treated with chemotherapeutic agent that may be at least one Cytotoxic antibiotics. The anthracyclines agents described above are also classified as “cytotoxic antibiotics”. Cytotoxic antibiotics also include the agent actinomycin D (also known generically as Actinomycin or Dactinomycin), which is the most significant member of the actinomycines class of polypeptide antibiotics (that were also isolated from streptomyces). Actinomycin D is shown to have the ability to inhibit transcription by binding DNA at the transcription initiation complex and preventing elongation of RNA chain by RNA polymerase. Other cytotoxic antibiotics include bleomycin, epirubicin and mitomycin.

Still further, in some embodiments the modulators used by the invention may be applicable for subjects suffering from immune-deficiency caused by immune-therapy or a biological therapy. More specifically, cancer vaccines, antibody treatments, and other “immunotherapies” are potentially more specific and effective and less toxic than the current approaches of cancer treatment and are generally termed “immunotherapy”, and therefore, an agent used for immunotherapy, is defined herein as an immuno-therapeutic agent. The term immunotherapy as herein defined (also termed biologic therapy or biotherapy) is a treatment that uses certain components of the immune system to fight diseases (e.g. cancer), by, inter alia, stimulating the immune system to become more efficient in attacking cancer cells (e.g., by administering vaccines) or by administering components of the immune system (e.g., by administering cytokines, antibodies, etc.).

In the last few decades immunotherapy has become an important part of treating several types of cancer with the main types of immunotherapy used being monoclonal antibodies (either naked or conjugated), cancer vaccines (i.e. that induce the immune system to mount an attack against cancer cells in the body) and non-specific immunotherapies.

Antibody-mediated therapy as referred to herein refers to the use of antibodies that are specific to a cancer cell or to any protein derived there-from for the treatment of cancer. As a non-limiting example, such antibodies may be monoclonal or polyclonal which may be naked or conjugated to another molecule. Antibodies used for the treatment of cancer may be conjugated to a cytotoxic moiety or radioactive isotope, to selectively eliminate cancer cells.

It should be noted that the term “biological treatment” or “biological agent”, as used herein refers to any biological material that affects different cellular pathways. Such agent may include antibodies, for example, antibodies directed to cell surface receptors participating in signaling, that may either activate or inhibit the target receptor. Such biological agent may also include any soluble receptor, cytokine, peptides or ligands. Non limiting examples for monoclonal antibodies that are used for the treatment of cancer include bevacizumab (also known as Avastin), rituximab (anti CD20 antibody), cetuximab (also known as Erbitux), anti-CTLA4 antibody and panitumumab (also known as Vectibix) and anti Gr1 antibodies.

More specifically, cancer vaccines as referred to herein are vaccines that induce the immune system to mount an attack against cancer cells in the body. A cancer treatment vaccine uses cancer cells, parts of cells, or pure antigens to increase the immune response against cancer cells that are already in the body. These cancer vaccines are often combined with other substances or adjuvants that enhance the immune response.

Non-specific immunotherapies as referred to herein do not target a certain cell or antigen, but rather stimulate the immune system in a general way, which may still result in an enhanced activity of the immune system against cancer cells. A non-limiting example of non-specific immunotherapies includes cytokines (e.g. interleukins, interferons). It should be thus appreciated that in some embodiments, the modulators used by the invention may be used as a combined supportive treatment for patients suffering from immune suppression. This supportive treatment may be combined with other supportive therapies as discussed herein.

Thus, in yet other embodiments, modulators used by the invention may be applicable for subjects undergoing at least one of adoptive cell transfer, a cancer vaccine, antibody-based therapy, a hormone, a cytokine or any combination thereof.

As indicated above, in some embodiments, the modulators of the invention may be used for boosting the immune response in subjects undergoing radiotherapy. Radiation therapy or radiotherapy, often abbreviated RT, RTx, or XRT, is therapy using ionizing radiation, generally as part of cancer treatment to control or kill malignant cells and normally delivered by a linear accelerator. Radiation therapy may be curative in a number of types of cancer if they are localized to one area of the body. It may also be used as part of adjuvant therapy, to prevent tumor recurrence after surgery to remove a primary malignant tumor (for example, early stages of breast cancer). According to some specific embodiment, the radiation is ionizing radiation, which may be any one of X-rays, gamma rays and charged particles. In other embodiments, the radiation may be employed in the course of total body irradiation, brachytherapy, radioisotope therapy, external beam radiotherapy, stereotactic radio surgery (SRS), stereotactic body radiation therapy, particle or proton therapy, or body imaging using the ionizing radiation.

As indicated above, in some embodiments, the modulators used by the invention may be used for boosting the immune response in subjects undergoing gene therapy. Gene therapy is the therapeutic delivery of nucleic acid polymers into a patient's cells as a drug to treat disease. The most common form uses DNA, optionally packed in a vector, that encodes a functional, therapeutic gene to replace a mutated gene.

It should be noted that the therapeutic methods disclosed by the invention may use any of the administration modes described herein before, in connection with the compositions of the invention, for example, administration by parenteral, intraperitoneal, transdermal, oral (including buccal or sublingual), rectal, topical (including buccal or sublingual), vaginal, intranasal and any other appropriate routes.

As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “effective amount” or “sufficient amount” used by the methods of the invention, mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

The terms “treat, treating, treatment” as used herein and in the claims mean ameliorating one or more clinical indicia of disease activity by administering a pharmaceutical composition of the invention in a patient having a pathologic disorder.

More specifically, the term “treatment”, as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

Still further, as mentioned above, the term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse re recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or even 100%.

With regards to the above, it is to be understood that, where provided, percentage values such as, for example, 10%, 50%, 100%, 120%, 500%, etc., are interchangeable with “fold change” values, i.e., 0.1, 0.5, 1.2, 5, etc., respectively.

The present invention relates to the treatment of subjects or patients, in need thereof. By “patient” or “subject in need” it is meant any organism who may be affected by the above-mentioned conditions, and to whom the monitoring and diagnosis methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the subject may be also any reptile or zoo animal. More specifically, the methods of the invention are intended for mammals By “mammalian subject” is meant any mammal for which the proposed therapy is desired, including human, livestock, equine, canine, and feline subjects, most specifically humans.

The invention further provides in another aspect thereof a modulator of hematopoietic cell activation. In more specific embodiments, the modulator of the invention may be characterized in that it modulates at least one of actin and myosin ARF in a cell. In some embodiments the modulator of the invention may modulate ARF in a lymphocyte cell forming an activating or inhibitory immunological synapse (IS). In some embodiments, such lymphocyte cell may be at least one of an NK cell, a T cell and a B cell forming an inhibitory or activating IS.

In some specific embodiments, the modulators according to the present invention are particularly applicable to a lymphocyte cell that is a NK cell forming an activating or inhibitory NK immunological synapse (NKIS).

As mentioned above, the inhibitory NKIS, wherein the synapse is in its nascent state, is of a particular interest for the present invention, as it is conceived. Thus, in specific embodiments, the modulators of the invention may be capable activating NK cells in inhibitory NKIS by inhibiting ARF in said NK cells.

In further embodiments, the activity of this type of modulators, i.e. those acting via the ARF inhibition, results in formation of a complex comprising β-actin and at least one phosphatase, specifically, at least one protein tyrosine phosphatase (PTP). In some specific embodiments, this PTP may be a SH2 domain-containing phosphatase in said NK cell. In some embodiments, such SHP may be at least one of SHP-1 and SHP-2. In some specific embodiments, such SHP is SHP-1.

Thus, in certain embodiments, activation of NK cells may result in phosphorylation of at least one of VAV1 and PLCγ1/2 and/or increase in intracellular calcium flux.

In numerous embodiments, the modulators according to the present invention are activating NK cells forming inhibitory NKIS which results in an increase in at least one of intracellular Ca²⁺ flux, and secretion of cytolytic granules in said NK cell. Thus, in some embodiments such activation may result in increased killing of target cells by the activated NK cells.

In further specific and non-limiting embodiments, the modulators according to the above may be any one of Jasplakinolide (JAS), the Rho kinase inhibitor of myosin light chain (MLC) phosphorylation (Y-27632), and Cyt D, any combination thereof or any vehicle, matrix, nano- or micro-particle comprising the same. It should be noted that in some embodiments, any of the compound disclosed by the invention and particularly, any of the compounds disclosed by Table 1, may be the modulator of lymphocyte activation in accordance with the invention.

In yet some further embodiments, the invention provides any modulator of lymphocyte cell activation with the proviso that such modulator is not Jasplakinolide (JAS), specifically as denoted by formula 12.

In yet some further embodiments, the invention provides any modulator of lymphocyte cell activation with the proviso that such modulator is not Y-27632, specifically as denoted by formula 35.

Still further, in some embodiments, the invention provides any modulator of lymphocyte cell activation with the proviso that such modulator is not Cyt D, specifically as denoted by formula 2.

In yet some further embodiments, the invention provides any modulator of lymphocyte cell activation with the proviso that such modulator is not any of the modulators disclosed in Table 1, specifically, any of the compounds of any one of Formulas 1 to 37.

In yet some further embodiments the invention encompasses any nanoparticle comprising any of the modulators of the invention. Some particular embodiments relate to any of the liposomes described herein above, as well as in EXAMPLE 9 and FIG. 15, specifically, nanoparticles composed of PC:DPPE:Chol, that may be coated with at least one of HA, anti-NKp46 antibody and LFA1, that comprise an effective amount of JAS. In some further specific embodiments, the invention provides nanoparticles composed of PC:DPPE:Chol, that may be coated with at least one of HA and anti-NKp46 antibody, that comprise an effective amount of Y-27. In yet some further specific embodiments, the invention provides nanoparticles composed of PC:DPPE:Chol, that may be coated with at least one of HA and anti-NKp46 antibody, that comprise an effective amount of CytD.

Still further, it is another important aspect of the present invention to provide a composition comprising an effective amount of a modulator of hematopoietic cell activation or any vehicle, matrix, nano- or micro-particle comprising the same. In more specific embodiments, the compositions of the invention may comprise modulators of lymphocyte activation, wherein said modulator is characterized in that it modulates ARF in a lymphocyte cell forming an activating or inhibitory IS. In certain embodiments, the composition may optionally further comprise at least one of pharmaceutically acceptable carrier/s, excipient/s, auxiliaries, and/or diluent/s. In some embodiments, such lymphocyte cell may be at least one of an NK cell, a T cell and a B cell forming an inhibitory or activating IS.

In specific embodiments, the compositions according to the invention, namely those comprising the relevant modulators as described above, are applicable to modulation of NK cells forming activating or inhibitory NKIS.

In further specific embodiments, the compositions according to the invention are applicable to modulation of NK cells forming inhibitory NKIS, wherein said modulator comprised within these compositions inhibits or disturbs ARF in NK cells.

In yet further specific embodiments, the compositions according to the invention, especially those inhibiting or disrupting ARF, lead to formation of complex comprising β-actin and at least one phosphatase, specifically, at least one PTP in said NK cell. In more specific embodiments, the composition of the invention may lead to formation of complex comprising β-actin and at least SH2 domain-containing phosphatase in said NK cell. In some embodiments, such SHP, may be at least one of SHP-1 and SHP-2.

In still further specific embodiments, the compositions according to the above lead to the formation of a specific complex between the SH2 domain-containing phosphatase SHP-1 and the β-actin, which thereby induces a change in the SHP-1 conformation and catalytic activity in NK cells.

In other words, the general purpose of the compositions of the invention is to modulate, meaning activate or inhibit, cytotoxic activity lymphocytes or NK cells forming in IS. In specific embodiments, the compositions are intended to activate nascent NK cells forming inhibitory NKIS.

In numerous embodiments, the compositions according to the invention, those that are activating of NK cells as above, lead to increase in at least one of intracellular Ca²⁺ flux, and secretion or formation of cytolytic granules in these NK cells.

Thus, in yet some further embodiments, the invention provides compositions that modulate the phosphorylation of VAV1. In yet some further specific embodiments, the invention provides the use of ARF inhibitors for increasing the phosphorylation of VAV1 in a cell.

In yet some further embodiments, the invention provides compositions that modulate the phosphorylation of PLCγ1/2. In yet some further specific embodiments, the invention provides the use of ARF inhibitors for increasing the phosphorylation of PLCγ1/2 in a cell.

Still further, the invention provides compositions comprising ARF inhibitors for increasing killing of target cells by NK cells.

As previous discussed, in specific embodiments the compositions according to the invention, especially those acting as ARF inhibitors, can comprise at least one inhibitor of actin depolymerization or an F-actin stabilizer, and/or an inhibitor of myosinIIA phosphorylation and/or myosinIIA activity.

In further specific embodiments such compositions can comprise an ARF inhibitor that is any one of JAS, CytD and Y-27632, or any derivatives or combinations thereof.

It should be appreciated that the invention further encompasses compositions comprising any of the modulators described herein or any derivatives, formulations or combinations thereof.

It should be noted that in all of the above numerous embodiments, the compositions of the invention may comprise one or more kind of the modulators of ARF or any nanoparticles thereof, specifically as provided by the invention, and optionally one or more additional known therapeutic agents to achieve the desirable level and kind of control on lymphocyte activation.

In yet some further embodiments, the invention provides a composition comprising any modulator of lymphocyte cell activation with the proviso that such modulator is not Jasplakinolide (JAS), specifically as denoted by formula 12.

In yet some further embodiments, the invention provides a composition comprising any modulator of lymphocyte cell activation with the proviso that such modulator is not Y-27632, specifically as denoted by formula 35.

Still further, in some embodiments, the invention provides a composition comprising any modulator of lymphocyte cell activation with the proviso that such modulator is not Cyt D, specifically as denoted by formula 2.

In yet some further embodiments, the invention provides a composition comprising any modulator of lymphocyte cell activation with the proviso that such modulator is not any of the modulators disclosed in Table 1.

A further aspect provides the use of at least one modulator according to the invention or of any vehicle, matrix, nano- or micro-particle comprising the same, in the preparation of a composition for treating, preventing, inhibiting, reducing, eliminating, protecting or delaying the onset of an immune-related disorder in a subject in need thereof.

Still further, the invention further provides in another aspect thereof, a vehicle, matrix, nano- or micro-particle comprising at least one modulator of hematopoietic cell activation, specifically, lymphocyte cell activation. In certain embodiments, the vehicle, matrix, nano- or micro-particle may comprise any of the modutators described by the invention.

In yet another aspect, the invention relates to a method for screening for a modulator of a NK cell activation. In more specific embodiments the method comprising the steps of: (a) contacting the NK cell with at least one of activating or inhibitory target cell or with a solid support coated with at least one of activating or inhibitory molecules (e.g., antibodies). In the next step (b) contacting the NK cell with at least one of activating or inhibitory target cell or with a solid support coated with at least one of activating or inhibitory molecules (e.g., antibodies) in the presence of a candidate modulator compound. The final step (c) involves determining at least one of: (i) F-actin accumulation in said NK cells of (a) and of (b); (ii) at least one of F-actin and myosin dynamics in said NK cells of (a) and of (b) and (iii) target cell lysis by said NK cells of (a) and of (b). It should be noted that a change in at least one of: F-actin accumulation as determined in step (c i), at least one of F-actin and myosin dynamics as determined in step (c ii) and target cell lysis as determined in step (ciii) in the presence of the tested candidate compound of (b) as compared to the absence of said compound of (a) indicates that the tested candidate compound modulates NK cell activation.

Other purposes and advantages of the invention will become apparent as the description proceeds. While in the foregoing description describes in detail only a few specific embodiments of the invention, it will be understood by those skilled in the art that the invention is not limited thereto and that other variations in form and details may be possible without departing from the scope and spirit of the invention herein disclosed or exceeding the scope of the claims.

The present invention as defined by the claims, the contents of which are to be read as included within the disclosure of the specification, and will now be described by way of example with reference to the accompanying Figures.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10%. The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range.

For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials and Reagents

Primary NK Cells

Primary NK cells were isolated from peripheral blood lymphocytes (PBLs) of healthy donors using the EasySep™ human NK Cell enrichment kit (STEMCELL Technologies). Subsequently, KIR2DL1 expressing cells were isolated by staining the entire NK cell population with the anti-KIR1-PE antibody (Miltenyi Biotec) and subsequent magnetic separation using the EasySep™ human PE selection kit (STEMCELL Technologies) according to the manufacturer's instructions. The NK cell isolation efficiencies were >95%. NK cells were plated in 96-well U-bottomed plates and grown in the presence of irradiated PBLs from two donors (5×10⁴ cells from each donor per well) as feeder cells. Cells were expanded in a complete medium containing 1 μg/ml of PHA, and 400 U/ml rhuIL-2 (Prospec). Cells were washed to remove the PHA and IL-2 and cultured in 60% Dulbecco's modified Eagle medium (DMEM) and 25% F-12 medium supplemented with 10% human serum, 2 mM L-glutamine, 50 μg/ml penicillin, 50 μg/ml streptomycin, 1% non-essential amino acids, and 1% sodium pyruvate.

Cell Lines

The YTS NK cell line expressing the inhibitory KIR2DL1 receptor (referred as YTS-2DL1), 721.221 B-cell lymphoma cells (referred to as 221), and 221 cells expressing either HLA-Cw4, -Cw6 or -Cw7 were obtained from the Department of Microbiology and Immunology, Faculty of Medicine, Hebrew University. YTS cells were cultured in Iscove's medium supplemented with 10% fetal bovine serum (FBS), 2 mM L-glutamine, 50 μg/ml penicillin, 50 μg/ml streptomycin and 50 μM 2-mercapto-ethanol. 221 cells were cultured in RPMI supplemented with 10% FBS, 2 mM L-glutamine, 50 μg/ml penicillin, 50 μg/ml streptomycin, 1% non-essential amino acids and 1% sodium pyruvate.

Antibodies and Reagents

Antibodies included: Mouse anti-GFP (Roche); Mouse anti-phosphoTyrosine 4G10, and Mouse anti-PLCγ1 (Upstate); Mouse anti-VAV1 (D7), Rabbit anti-SHP-1, Rabbit anti-PLCγ2, and Mouse anti-β-actin (Santa Cruz); Mouse anti-GAPDH (Biodesign); Rabbit anti-pPLCγ2 (Y1217); Rabbit anti-phospho-Myosin light chain 2 (S19) (Cell Signaling); Rabbit anti-pPLCγ1 (Y783); Rabbit anti-pVAV1 (Y160) (Bio Source); Mouse anti-CD107a, Mouse anti-CD28, Mouse anti-KIR2DL/SD1 (BioLegend); Mouse anti-NKG2A, Mouse anti-NKG2D (R&D systems); Mouse anti-KIR2DL/SD1 PE (Miltenyi Biotec). Secondary antibodies: Goat anti-Mouse (Sigma-Aldrich), Goat anti-Rabbit (Santa Cruz), Alexa Fluor-conjugated antibodies (Molecular Probes). Reagents included: Y-27632 (Cayman Chemical); Jasplakinolide (Santa Cruz); pNPP (New England BioLabs).

Experimental Procedures

DNA Constructs and Mutagenesis

The human SHP-1 wt cDNA was obtained from Addgene; the Myosin IIA heavy chain cDNA—from the Department of Biochemistry and Molecular Biology, Faculty of Medicine, Hebrew University; and the F-tractin cDNA from the Department of Molecular Cell Biology, Faculty of Biology, Weizmann Institute of Science. The cDNAs of SHP-1, actin, and F-tractin were cloned into the expression vectors pEYFP-C1, pECFP-N1, pECFP-C1, pEGFP-N1 (Clontech) or pmCherry [14], to obtain the chimeric proteins, CFP-actin, F-tractin GFP, YFP-SHP-1-CFP, mCherry-MyosinIIA, or YFP-SHP-1. To avoid localization of SHP-1 to the nucleus, YFP-SHP1-CFP was mutated at its NLS sequence. A A206K substitution was used to render Aequorea GFP derivatives monomeric [12]. Molecular mutants were prepared using the QuikChange II XL site-directed mutagenesis kit (Stratagene).

CRISPR/CAS9 Gene Knockout

CRISPR/CAS9 mediated knockout of endogenous SHP-1 in YTS-2DL1 cells was conducted as previously described (Ran et al, 2013). Custom Single Guide RNAs (sgRNAs) were subcloned into the vector pSpCas9(BB)-2A-GFP (PX458) (a gift from Feng Zhang; Addgene plasmid #48138), which encodes for the CAS9 nuclease. sgRNA sequences aimed for SHP-1 locus were constructed using the online CRISPR design tool (Zhang Lab) and NCBI gene. sgRNA sequences are as follows:

Top: 5′-CACCgTCGGCCCAGTCGCAAGAACC-3′; as denoted by SEQ ID NO: 17.

Bottom: AAACGGTTCTTGCGACTGGGCCGAc, as denoted by SEQ ID NO: 18.

2*10⁶ YTS-2DL1 cells were transfected with Nucleofector 2b (Lonza) with Amaxa solution R and protocol X-001. After transfection, cells were incubated at 37° C. and 5% CO₂ for 48 hours and were sorted according to GFP signal to collect only the cells transfected by the pSpCas9 (BB)-2A-GFP vector. Sorted cells were seeded into a 96 wells plate at a concentration of 1 cell per well. After cell growth, individual colonies were screened via western blot analysis using anti-SHP-1 antibody.

Cell Transfection and FACS Analysis

Primary NK cells were transfected with an AMAXA electroporator using AMAXA primary NK solution and protocol X-001. YTS-2DL1 or 221 cells were transfected using AMAXA solution T and protocol H-10. Transiently transfected cells were used after 24-48 h. Stable clones were derived from transiently transfected cells by a combination of drug selection and cell sorting. Cells transiently expressing chimeric proteins were selected in either neomycin, hygromycin or zeocin. Fluorescence analysis and cell sorting were performed using FACSAria (Becton Dickinson Biosciences).

Cell Stimulation, Immunoblotting and Immunoprecipitation

NK cells (primary or YTS) and ‘221’ target cells (either expressing HLA-Cw4, Cw7 or no HLA) were first incubated separately on ice for 10 min, at a ratio of 1:1. The cells were then mixed, centrifuged and kept on ice for 15 min. The cell mixture was then transferred to 37° C., for the indicated period of time, and subsequently lysed with ice-cold lysis buffer.

For analysis of whole cell lysates, 1-10×10⁵ cells were used, and for IP experiments, 10-15×10⁶ cells were used. Protein A/G plus-Agarose beads (Santa Cruz Biotechnology) were used for IP. Protein samples were resolved with sodium dodecyl sulfate-polyacrylamide gel electrophoresis (SDS-PAGE), transferred to nitrocellulose membrane, and immunoblotted with the appropriate primary antibodies. Immunoreactive proteins were detected with either anti-mouse or anti-rabbit horseradish peroxidase-coupled secondary antibody followed by detection by enhanced chemiluminescence (PerkinElmer).

PTP Assay

SHP-1 catalytic activity was determined by measuring the hydrolysis of the exogenous substrate p-Nitrophenyl Phosphate (pNPP) by SHP-1, as previously described [13]. NK cells (2-5×10⁶) were incubated with target cells at ratio of 1:1, activated as described above, and lysed with passive ice-cold lysis buffer (1% Brij, 1% n-Octyl-b-D-glucoside, 50 mM Tris-HCl, pH 7.4, 150 mM NaCl, 5 mM ethylenediaminetetraacetic acid, and complete protease inhibitor tablets). Cell lysates were subjected to IP with anti-SHP-1 antibody. Immunoprecipitates were washed twice with ice-cold passive washing buffer (0.1% Brij, 50 mM Tris-HCl, pH 7.4, 300 mM NaCl, and 3.75 mM ethylenediaminetetraacetic acid), and three times with phosphatase buffer (150 mM NaCl, 50 mM HEPES, 1 mM ethylenediaminetetraacetic acid and 1 mM DDT) Immunoprecipitates were resuspended in 200 μl 25 mM pNPP in phosphatase buffer (1 mg/ml BSA, 25 mM HEPES [pH 7.2], 50 mM NaCl, 2.5 mM EDTA, and 10 mM DTT) and incubated for 30 min at 37° C. Reactions were terminated by adding 800 μl 1M NaOH, and SHP-1 activity was determined by measuring absorbance at 405 nm.

Measurement of Intracellular Calcium Concentration

NK cells (0.5-1×10⁶) were incubated with 5 μM Fluo-3-acetoxymethylester (Fluo-3-AM, Biotium) in RPMI 1640 medium at 37° C. for 45 min. The cells were washed twice, resuspended in RPMI 1640 without phenol red and maintained at room temperature for 20 min. The cells were incubated at 37° C. for 5 min before measurement, then mixed at a 1:1 ratio with 221 target cells, and the Ca²⁺ influx was measured spectrofluorometrically using the SpectraFluor Plus Microplate Reader (TECAN).

CD107a Degranulation Assay

3×10⁵ YTS cells or primary NK cells were co-incubated with 6×10⁵ 221 target cells expressing mCherry at 37° C. for 2 hours in the presence of 2 μM monensin (BioLegend). The cells were centrifuged, incubated with diluted anti-CD107a for 30 minutes on ice and washed twice. Cells were then stained with isotype-specific AlexaFluor-conjugated antibody on ice for 30 minutes. Cells were washed twice and analyzed by FACS. YTS or primary NK cells were distinguished from target cells according to mCherry expression by targets.

Granzyme B Secretion Analysis

Granzyme B release was measured by ELISA. NK cells (2×10⁵) were washed with PBS twice and incubated in 200 μl complete medium with target cells (4×10⁵) for 2 h at 37° C., 5% CO₂. Following 5 min of incubation, 1 μM JAS was added. Cells were centrifuged, and supernatants were collected and stored at −20° C. Release of Granzyme B into supernatants was quantified by ELISA (MABTECH).

Confocal Microscopy

Dynamic fluorescent and differential interference contrast microscopy (DIC) images were obtained using a Zeiss 510 Meta confocal microscope. All images were collected using a 63 X Plan-Apochromat objective (Carl Zeiss) and were extracted with the LSM browser (Carl Zeiss).

Live Cell Microscopy

Live cell movies were obtained using Zeiss Observer.Z1 equipped with a Plan Apochromat×100/1.4 NA oil objective and ORCA-ER digital camera (Hamamatsu). Movies were collected with 500 ms exposure at a frame interval of 1 sec, for the indicated time, in 5% CO₂, at 37° C. Stacks were displayed as maximum intensity projections.

Cellular Imaging

Chambered cover glasses (LabTek) were cleaned by treatment with 1 M HCl, 70% ethanol for 30 min and dried at 60° C. for 30 min. The chambers were treated with a 0.01% (wt/vol) poly-L-lysine solution (Sigma) for 5 min, drained, and dried at 60° C. for 30 min. For NK-target conjugation assays, 5×10⁵ target cells were seeded over the bottom of the chamber in 300 μl Optimem medium for 2 hours at 37° C., after which nonadherent cells were washed off. Then, 5×10⁵ NK cells were seeded over the chambers, containing imaging buffer (RPMI medium with 25 mM HEPES without phenol red or serum), and allowed to form conjugates with the target cells for the indicated times at 37° C. Following activation, the cells were fixed for 30 min with 2.5% paraformaldehyde and washed twice with PBS. The NK and target cells in the conjugates were distinguished based on fluorescence signal, in which the target cells expressed mCherry and the NK cells expressed various YFP, CFP or GFP tagged proteins. For 3D projection of the NKIS plane, 20 μm Z stacks of NK-target conjugates were collected, at 0.5 μm intervals, and NKIS images were assembled by Imaris (version X64 7.7.2). For evaluation of phospho-protein accumulation at the NKIS, cells were permeabilized with 0.1% Triton X-100 for 5 min. Cells were blocked for 1 h in PFN buffer (PBS without Ca²⁺ and Mg²⁺ and containing 10% FCS and 0.02% azide) and 2% normal goat serum (Jackson ImmunoResearch). Cells were incubated with the indicated primary antibodies diluted in blocking medium for 1 h, followed by staining with isotype-specific AlexaFluor-conjugated antibody for 30 min. Cells were washed three times with PFN between steps. The relative fluorescence intensity of the proteins at the synapse was determined by measuring the ratio between fluorescence intensity at the NKIS relative to a non-NKIS site using ImageJ. For imaging of fixed NK cells spreading over antibody coated surfaces, poly-L-lysine coated chambers were coated with activating or inhibitory antibodies (10 μg/ml) overnight at 4° C., using anti-NKG2D or anti-NKG2A for primary NK cells, or anti-CD28 or anti-KIR2DL/DS1 for YTS cells. Excess antibody was removed by extensive washing with PBS. Cells (5×10⁵) were seeded over the bottom of chambers containing 300 μl imaging buffer for the indicated times, and then were fixed for 25 min with 2.5% paraformaldehyde in PBS. Images of a single confocal plane at the contact interface were collected. Calculation of the NK cell contact site area was performed using ImageJ. Boundaries of F-tractin-GFP expressing cells were identified by thresholds of the GFP signal, automatically set by the ImageJ software. All images were cropped and composed into figures within Adobe Photoshop.

Analysis of Actin and Myosin Distribution

Analyses of actin and myosin distribution along the diameter of the immune synapse were performed by measuring the florescence intensity along virtual lines stretching from one point at the cell's periphery, through the center, to the other side of the cell. Two perpendicular lines were drawn and calculated for each imaged cell. Cell diameter was normalized to 1, and results from each sampled group were divided into 100 bins representing their relative location along the diameter of the synapse. For each bin, the average intensity was calculated and normalized using the average intensity of each measured line to determine F-actin fold intensity along the diameter. All actin and myosin distribution analyses were performed using ImageJ. PERL and Microsoft Excel were used for data binning and statistics.

Protein Dynamics Image Analysis

Analyses of actin and SHP-1 dynamics were performed by preparing kymographs from live cell videos and tracing the movement of the fluorescently tagged proteins. These data of protein traces were then used for calculating protein dynamics. To facilitate identification of F-actin speckles, kymographs were processed using the Unsharpen Mask filter in ImageJ (with values of radius 5 and mask weight 0.8). Velocity over distance from the center was determined by calculating the velocity of the tracked protein based on the distance traversed to time between each two points on the trace, and noting the relative location from the center of the measured area. NKIS radius was normalized to 1 and data points were then split into bins according to their relative cellular location. For each bin, average velocity and standard error were calculated. Average distance traversed over time was calculated by measuring the location of the trace, relative to its point of origin at each time point after the beginning of the trace. For each relative time point from the beginning of a trace, an average distance and standard error were calculated. Velocity over time of spreading was calculated by measuring the velocity of the tracked protein as described above, while noting the time after the beginning of cellular spreading. Data points were then split into bins according to the time index. For each bin, average velocity and standard error were calculated. All protein dynamics image analyses were performed using ImageJ. PERL and Microsoft Excel were used for data binning and statistics.

Preparation of Polyacrylamide Gel Substrates

Polyacrylamide gel substrates were prepared with various ratios of 40% acrylamide and 2% bisacrylamide to attain desired stiffness, according to a previously described protocol (Tse J R, et al (2010) Current protocols in cell biology Chapter 10: Unit 10-16). Gel substrates were attached to the glass bottom of a CELLview 35-mm dish (Greiner Bio One), coated with 0.01% Poly-1-lysine, using the hydrazine hydrate method, according to a previously described protocol (Hui K L et al (2015) Molecular biology of the cell 26: 685-695), and incubated with 10 μg/ml anti-CD28 or anti-KIR2DL1 antibodies at 4° C. overnight. Gel substrates with different levels of stiffness were fabricated using the following acrylamide/Bisacrylamide concentrations (Tse J R, et al (2010) Current protocols in cell biology Chapter 10: Unit 10-16): 5% and 0.03% for 1 kPa gels, and 12% and 0.575% for 50 kPa gels.

NK Cell Killing Assay

Chambered cover glasses (LabTek) were covered with 0.2% (wt/vol) gelatin (BioRad) (dissolved in 2% sucrose solution) and dried at 25° C. for 3 hours. The target cells were stained with 3 μM calcein-AM (Molecular Probes) in RPMI 1640 for 30 min at 37° C., and then washed twice prior to experimental procedures. Then, 5×10⁵ target cells were seeded over the bottom of the chamber in 300 μl imaging buffer and incubated for 2 hours at 37° C., after which non-adherent cells were washed off. Next, 5×10⁵ NK cells were seeded over the chambers containing the target cells in imaging buffer, allowed to form conjugates for 5 minutes at 37° C., and treated with 1 μM JAS or left untreated. After that, 16.8 μm Z stacks of target cells were collected at 2.1 μm intervals every 2 minutes for a total of 2 hours at 37° C., 5% CO₂, using Leica SP8 confocal microscope with a Plan Apochromat×40/1.30 oil objective. Movies were prepared from z-stacks by making a maximum-intensity projection for a given time point and then compiling a sequence of all the projections. Movie analyses of the calcein fluorescence intensity of target cells over time were performed using ImageJ. Boundaries of calcein-labeled target cells were defined by thresholds of the fluorescent signal, automatically set by the ImageJ software, and the fluorescence intensity of cells was examined manually at the indicated time points. The relative calcein fluorescence intensity was determined by measuring the ratio between the fluorescence intensity at time=120 min relative to the fluorescence intensity at time=0 min. The results are presented relative to the florescence measured in the ‘Target cell only’ sample.

FRET (Förster or Fluorescence Resonance Energy Transfer) Analysis

FRET was measured by the donor-sensitized acceptor fluorescence technique. Briefly, three sets of filters were used: one optimized for donor fluorescence (excitation, 458 nm, and emission, 465 to 510 nm), a second for acceptor fluorescence (excitation, 514 nm, and emission, 530 to 600 nm), and a third for FRET (excitation, 458 nm; emission, 530 to 600 nm).

FRET Correction

FRET correction was performed as previously described. In brief, the non-FRET components were calculated and removed using calibration curves derived from images of single-labeled CFP- or YFP-expressing cells. Sets of reference images were obtained using the same acquisition parameters as those used for the experimental FRET images. To correct for CFP “bleed through,” the intensity of each pixel in the CFP image from CFP-expressing cells was compared to the equivalent pixel in the FRET image of the same cells. A calibration curve was derived that defined the amount of CFP fluorescence seen in the FRET image as a function of the fluorescence in the CFP image. A similar calibration curve was obtained defining the amount of YFP fluorescence appearing in the FRET image as a function of the intensity in the YFP image, using images of cells expressing only YFP. Separate calibration curves were derived for each set of acquisition parameters used in the FRET experiments. Then, using the appropriate calibration curves, together with the CFP and YFP images, the amount of CFP bleed through and YFP cross excitation were calculated for each pixel in the experimental FRET images. These non-FRET components were subtracted from the raw FRET images, yielding corrected FRET images.

FRET Efficiency Calculation

The FRET efficiency (FRETeff) was calculated on a pixel-by-pixel basis using the following equation: FRETeff=FRETcorr/(FRETcorr+CFP)×100%, where FRETcorr is the pixel intensity in the corrected FRET image, and CFP is the intensity of the corresponding pixel in the CFP channel image. To increase the reliability of the calculations and to prevent low-level noise from distorting the calculated ratio, pixels below 50 intensity units and saturated pixels were excluded from the calculations and their intensities set to zero. These pixels are shown in black in the “pseudocolored” FRET efficiency images. To estimate the significance of the FRET efficiency values obtained, and to exclude the possibility of obtaining false-positive FRET results, cells expressing free CFP and free YFP were prepared as negative controls. The FRET efficiency in the negative-control system was measured and calculated in the same way as in the main experiment. FRET efficiency values obtained from the negative-control samples were subtracted from the values obtained in the main experiments. Image processing and measurements were performed using IPLab software (version 3.9).

MST Measurements

YFP-tagged SHP-1 was obtained from cell lysates of Human Embryonic Kidney (HEK) 293T cells, transiently transfected with YFP-SHP-1 by DNA-calcium phosphate co-precipitation, as previously described (Reicher B, et al (2012) Molecular and cellular biology 15: 3153-3163). MST measurements were performed using the protein purification-free method described by Khavrutskii et al. (Khavrutskii L, et al (2013) Journal of visualized experiments: JoVE). Briefly, each peptide (Synpeptide) was initially dissolved in Dimethylformamide (DMF), diluted in DDW (supplemented with 1 mM DTT) to reach stock concentration of 80004, and serially diluted (range from 400 μM to 48.8 nM) in HEPES buffer (50 mM, supplemented with 0.05% Tween-20 and 1 mM DTT) in 200 μL PCR-tubes. Then, YFP-SHP-1-containing cell lysate was added to the serially diluted tubes at a 1:1 ratio and the samples were gently mixed. The samples were allowed to incubate at room temperature for 30 min before being loaded into standard-treated Monolith™ capillaries (NanoTemper). After loading into the instrument (Monolith NT.115, NanoTemper), microthermophoresis was carried out using 40% LED power and 40% MST power. The changes of the fluorescent thermophoresis signals were plotted against the concentration of the serially diluted peptides. K_{D} values were determined using the NanoTemper analysis software (MO.Affinity Analysis v2.1.3).

Mass Spectrometry—in-Gel Digestion and Protein Identification by LC-ESI-MS/MS

Protein bands were excised from an SDS gel stained with Coomassie blue. The protein bands were subsequently reduced, alkylated and in-gel digested with bovine Trypsin (Promega), at a concentration of 12.5 ng/μl in 50 mM ammonium bicarbonate at 37° C., as described (Shevchenko A, et al (1996) Analytical chemistry 68: 850-858). The peptide mixtures were extracted with 80% CH3CN, 1% CF3COOH, and the organic solvent was evaporated in a vacuum centrifuge. The resulting peptide mixtures were reconstituted in 80% formic acid and immediately diluted 1:10 with Milli-Q water before analysis. Liquid chromatography-tandem mass spectrometry (LC-MS/MS) was performed using a 15 cm reversed-phase fused-silica capillary column (inner diameter, 75 μm) made in-house and packed with 3 μm ReproSil-Pur C18AQ media (Dr. Maisch GmbH). The LC system, an UltiMate 3000 (Dionex), was used in conjunction with an LTQ Orbitrap XL (Thermo Fisher Scientific) mass spectrometer operated in the positive ion mode and equipped with a nanoelectrospray ion source. Peptides were separated with a 2 hour gradient from 5 to 65% acetonitrile (buffer A, 5% acetonitrile, 0.1% formic acid and 0.005% TFA; buffer B, 90% acetonitrile, 0.2% formic acid and 0.005% TFA). The voltage applied to the union to produce an electrospray was 1.2 kV. The mass spectrometer was operated in the data-dependent mode. Survey mass spectrometry scans were acquired in the Orbitrap with the resolution set to a value of 60,000. The seven most intense ions per scan were fragmented and analyzed in the linear ion trap. Raw data files were searched with MASCOT (Matrix Science) against a Swissprot database. Search parameters included a fixed modification of 57.02146 Da (carboxyamidomethylation) on Cys, and variable modifications 15.99491 Da (oxidation) on Met, and 0.984016 Da (deamidation) on Asn and Gln. The search parameters also included: maximum 2 missed cleavages, initial precursor ion mass tolerance 10 ppm, fragment ion mass tolerance 0.6 Da. Samples were further analyzed in Scaffold (Proteome software).

Statistical Analyses

Standard errors (SE) and significances were calculated using Microsoft Excel. Statistical significances were calculated with Student's t tests used for unpaired, two-tailed samples. In all cases, the threshold P value required for significance was 0.05.

Liposomal Nanoparticle (NP) Preparation

Multilamellar liposomes, composed of phosphatidylcholine (PC), dipalmitoyl phosphatidyl-ethanolamine (DPPE), and cholesterol (Chol) at molar ratios of 3:1:1 (PC:DPPE:Chol) were prepared by a lipid-film method. To enable detection of the NPs, DPPE labeled with Rhodamine red (DPPE-PE) were incorporated into the lipid mixture. The lipid film was hydrated to create multilamellar liposomes. Following 7 cycles of rapid freezing-thawing, using liquid nitrogen and a thermo-block set to 65° C., the multilamellar liposomes were extruded into unilamellar nano-scale liposomes (ULNL) with a hand-operated Mini-extruder™ device (Avanti Polar Lipids, Inc.). ULNL were surface-modified with high molecular weight Hyaluronan (HA) (>950 kDa, R&D Systems) and separated by ultra-centrifugation. The HA-modified NPs were coupled to approximately 25 μg of NK cell-specific antibody, NKp46, using an amine-coupling method. Particle diameters and surface charges (zeta potential) were measured during the various stages of their preparation. Anti-LFA1 coated NPs were examined for selectivity. The uptake of NPs coated with HA (CD44 ligand) by NK cells was further examined.

### Example 1

SHP-1 Binds to β-Actin During the NK Inhibitory Response

To characterize the role of actomyosin network in regulating NK cell signaling, the inventors examined the interaction between PTP SHP-1 and cytoskeletal and signaling molecules in inhibitory vs. activating NK cell states. YTS NK cells expressing the inhibitory receptor KIR2DL1 (YTS-2DL1 cells purification in FIG. 1A) were incubated with 721.221 target cells expressing the inhibitory HLA-Cw4 (221-Cw4 cells) haplotype, or the irrelevant HLA-Cw7 haplotype related to NK cell activation (221-Cw7 cells). The association of SHP-1 with tyrosine phosphorylated signaling proteins was determined by immunoprecipitation (IP) of SHP-1, revealing a prominent 42 kDa band upon inhibitory interaction (FIG. 2A, indicated by arrow). Mass-spectrometric analysis identified this band as β-actin (sequence thereof is presented in FIG. 1B, as also denoted by SEQ ID NO. 16), suggesting that SHP-1 interacts with the actin machinery during the inhibitory NK cell response. The mass-spectrometry data were confirmed by blotting the membrane with an antibody specific to β-actin (FIG. 2A), and were further validated by identifying an SHP-1: β-actin complex in KIR2DL1-expressing isolated primary NK cells incubated with inhibitory 221-Cw4 target cells vs. 721.221 activating target cells (pNK-2DL1 cells, FIG. 2B; for NK cell purification, FIG. 1C). To eliminate the possibility of unspecific binding of β-actin to A/G agarose beads, cell lysates were either incubated with the A/G agarose beads without antibody (FIGS. 2A and 2B; No Ab), or with beads attached to an irrelevant IgG isotype control antibody (FIG. 1D), demonstrating the lack of co-immunoprecipitation in the control samples. Moreover, SHP-1 was immunoprecipitated from cell lysates of YTS cells alone or target cells alone, demonstrating the lack of binding of SHP-1 to β-actin in the control samples (FIG. 1E). These data suggest that the SHP-1: β-actin complex is formed following NK cell inhibition, rather than disassociating following activation.

To further support the findings of SHP-1-β-actin interaction following NK cell inhibition, two additional experimental approaches were used. The first was analysis of Fluorescence Resonance Energy Transfer (FRET) efficiency between SHP-1 and β-actin to measure their complex formation on the nanometer scale. YTS-2DL1 cells stably expressing CFP-actin (YTS CFP-actin cells) and transfected with YFP-SHP-1 were examined following interaction with inhibitory 221-Cw4 or activating 221-Cw7 target cells expressing mCherry. FRET analysis demonstrated a significantly higher binding of SHP-1 to β-actin at the inhibitory vs. activating NKIS (18.9±3.6% vs. 8.5±2.7%; P=0.02; FIG. 1F1-3), supporting the biochemical data.

The SHP-1-β-actin complex formation was also examined using microscale thermophoresis (MST) technology. The binding of SHP-1 to a WT β-actin derived peptide that contains an ITIM motif (KEKLCYVALDF as denoted by SEQ ID NO 1) was measured. The binding of SHP-1 to a single aa mutant form of the β-actin peptide, containing a tyrosine to phenylalanine substitution (Y-F mutant), or irrelevant control peptide was also determined. Lysates of HEK 293T cells transiently expressing YFP-SHP-1 were incubated with decreasing concentrations of the different peptides followed by MST analysis. Strikingly, the WT actin peptide bound SHP-1 with a dissociation constant (K_{D}) of 39.5±6.2 μM, whereas no binding was detected with the mutant form or irrelevant peptides (FIG. 1G).

All together, these data indicate a direct interaction of SHP-1 with β-actin, specifically following NK cell inhibition, suggesting a possible role of the actin network in inhibitory signaling cascades.

To elucidate the role of actin in SHP-1 signaling, a pharmacological inhibitor was used. Jasplakinolide (JAS), a pharmacological inhibitor of actin turnover, acts by blocking F-actin depolymerization, depletion of the cellular G-actin pool, and stabilization of actin filaments. JAS was reported to inhibit actin dynamics and specifically ARF in several systems [8, 9]. The effect of JAS on the SHP-1/13-actin complex was determined in NK cells upon interaction with inhibitory or activating target cells. Surprisingly, JAS substantially increased SHP-1 binding to β-actin upon an inhibitory interaction, whereas no binding was detected upon activation, with or without JAS (FIG. 2C), thus suggesting possible involvement of ARF in SHP-1 signaling.

### Example 2

Inhibitory Vs. Activating NKISs are Characterized by Distinct F-Actin Distributions

To examine F-actin and myosin IIA distribution at the NKIS site, the inventors used YTS-2DL1 cells stably expressing F-tractin GFP (FIGS. 3A, 3B) and transiently expressing mCherry-myosin IIA. F-tractin GFP is an ideal reporter for visualizing F-actin organization and dynamics, since it neither affecting depolymerization rate of actin filaments nor interfering with the formation of different F-actin structures. Cells were seeded over coverslips pre-coated with a stimulatory antibody—anti-CD28, or an inhibitory antibody—anti-KIR1. Profiling of F-actin and myosin revealed distinct F-actin distributions in the activated vs. inhibited NK cells (FIG. 3C graph, shaded regions). F-Actin was preferentially accumulated at the peripheral region, of the activating NKIS forming a ring-like structure (FIG. 3C, upper cell images). This region is known as lamelliopodia (LP) of NKIS, i.e. cellular structures found at the leading edge of motile and spreading immune cells characterized by thin, sheet-like membrane protrusions containing dense and dynamic F-actin network. In inhibitory NKIS, actin demonstrated a dispersed distribution (lower image). In contrast, myosin IIA was accumulated at the lamellum (LM) and cell body (CB) regions, in both the inhibitory and activating systems (FIG. 3C, cell images and graph). LM is located behind LP and is composed of condensed linear actin network that is more stable and characterized in that it comprises the motor protein myosin II. CB is located behind LM and is generally lacking actin filaments.

These findings were further reproduced in NK-target cells conjugates, wherein YTS F-tractin GFP-expressing cells were incubated with mCherry expressing 221-Cw4 or 221-Cw7 cells. 3D projections assembly at the NKIS plane showed formation of F-actin ring-like structure in the activating NKIS compared to a dispersed distribution at the inhibitory NKIS (FIG. 3D), supporting the notion of differential F-actin organization in the inhibitory vs. activating NKIS.

### Example 3

Actin Dynamics Control SHP-1 Movement at the Inhibitory Vs. Activating NKIS

To address the question of actin movement and its spatial-temporal dynamics at the NK cell effector function, the inventors examined the actin centripetal retrograde flow (ARF) upon NK cell inhibition or activation in live cells. Imaging of F-actin dynamics was performed at the contact site of fully spread NK cells. To that end, YTS-2DL1 or primary NK cells expressing F-tractin GFP were seeded over surfaces pre-coated with stimulatory antibodies—anti-CD28 or NKG2D, or inhibitory antibodies—anti-KIR2DL1 or NKG2A (for F-tractin GFP expression level in pNK cells see FIG. 3E). Monitoring F-actin flow revealed that while the F-actin network in the LP region of activating NKIS demonstrated fast and continuous retrograde flow, in the inhibitory NKIS such continuous flow was barely detected, and instead random and inconsistent F-actin movements were observed.

To further characterize actin dynamics, ARF velocity was quantified using kymograph analysis along the radius of F-tractin expressing NK cells. F-actin features were monitored at the outer margin of kymographs representing LP, or at the intermediate and inner regions representing LM and CB (FIGS. 4A, 4B, bright and dark traces). The angle of F-actin trace is related to velocity, whereby vertical orientation indicates slow or negligible velocity, and horizontal orientation indicates fast velocity. Surprisingly, ARF at LP of both YTS and pNK cells was faster in the activating vs. inhibitory NKIS (FIGS. 4C, 4D; YTS 0.13±0.0037 μm/sec, pNK 0.2±0.006 μm/sec, and YTS 0.024±0.0014 μm/sec, pNK 0.017±0.0009 μm/sec, respectively, P≤0.00001). Under inhibitory conditions, the ARF average at LP showed a shallower slope (FIGS. 3F, 3G), suggesting significantly slower movement. Furthermore, these analyses suggested that the velocity of F-actin depends on its location along the NKIS radius. Kymograph analysis of vertical traces at LM and CB indicated an immobile F-actin network (FIG. 4A, dark signals). Further quantitative analysis showed a slower or negligible ARF at these regions (FIGS. 4C, 4D and FIGS. 3H, 3I). As a negative control, YTS F-tractin GFP cells were seeded over slides coated with IgG isotype antibody, followed by analysis of ARF velocity. Live cell imaging and kymograph analysis demonstrated negligible actin flow velocity, regardless of the location across the NKIS radius, which was significantly slower than ARF velocity at the LP of the inhibitory NKIS (IgG: 0.006±0.0004 μm/sec, KIR2DL1: 0.024±0.0014 μm/sec; P≤0.00001; FIG. 3J).

These results indicate differential distribution and F-actin dynamics at the activating vs. inhibitory NKIS. Increased F-actin accumulation was measured at the periphery of activating NKIS, with rapid ARF movement at the LP site, compared to inhibitory NKIS.

The inventors further investigated whether slower ARF at the inhibitory NKIS is related to the increased formation of the SHP-1/β-actin complex described above, in other words, whether ARF and SHP-1 dynamics are inter-related. A live-cell imaging of SHP-1 dynamics was performed in YTS-2DL1 cells transiently expressing mCherry-SHP-1 that were seeded over activating or inhibitory coverslips. Quantitative kymograph analysis indicated faster SHP-1 retrograde flow at LP of activating vs. inhibitory contact sites (FIGS. 4E, 4F and 3K; 0.15±0.0076 μm/sec vs. 0.021±0.0028 μm/sec, respectively, P≤0.00001). Similar velocity rates of ARF and SHP-1 suggested that the F-actin and SHP-1 translocations are inter-related.

### Example 4

F-Actin Turnover and myosinIIA Contractile Force are Required for F-Actin Flow

The inventors hypothesized that ARF could be potentially driven by F-actin polymerization, resulting in “pushing” forces towards the leading edge of a spreading cell, and/or myosin contractile forces that “pull” the F-actin network away from the cell membrane. To dissect the role of ARF driving forces in NK cells, pharmacological inhibitors were used. The role of actin turnover in this process was determined by examining the effect of JAS on ARF at the contact site of both YTS and pNK F-tractin GFP cells. Cells were seeded over activating and inhibitory surfaces, and after complete NK cell spreading JAS was added. These experiments showed that administration of JAS resulted in a rapid ARF decrease at LP (FIGS. 5A, 5B). Subsequent quantitative analysis showed that this ARF decrease started within ˜20 sec and reached full arrest by ˜150 sec (FIGS. 5C, 5D).

To further determine the role of actin polymerization in driving ARF in NK cells, the actin polymerization inhibitor, Cytochalasin D (CytD) was used, which was previously shown to decelerate actin dynamics and retrograde flow [9]. YTS F-tractin GFP cells were seeded over slides coated with anti-CD28 or anti-KIR2DL1 antibodies, and CytD was added to the cells following their spreading. Kymograph analysis at the LP demonstrated a significant reduction in ARF velocity upon CytD treatment, under both activating and inhibitory settings (FIG. 6), further supporting the key role of actin polymerization in driving ARF in NK cells.

Further, the role of myosinIIA in ARF dynamics was examined using Y-27632 (Y-27), i.e. Rho kinase inhibitor preventing myosin light chain (MLC) phosphorylation on Serine 19, and thereby disrupting the formation of myosin II filaments. YTS F-tractin GFP cells were treated with Y-27, and myosin activity was assessed using IB with anti-pMLC(S19) antibody. The results showed significant reduction in phosphorylation (FIG. 7). While monitoring ARF under these conditions in the activating vs. inhibitory NKIS, the inventors observed complete arrest of F-actin flow, even under full spreading (FIGS. 5E and 5G). Interestingly, in the Y-27 treatment the inventors noticed significant differences in the cell area, a significantly enlarged NKIS area was detected upon inhibition of myosinIIA activity in both, activating and inhibitory states. This finding suggests that myosinIIA antagonizes NK cell spreading by exerting contractile forces, while JAS had no such effect on the NK contact area (FIGS. 5F and 5H). The results of these pharmacological treatments suggested that actin polymerization and myosin contractile forces regulate F-actin flow in NK cells.

### Example 5

ARF Regulates SHP-1 Conformational State and Catalytic Activity

The NK inhibitory response involves recruitment of PTP SHP-1 to NKIS, where it binds and dephosphorylates signaling molecules such as actin regulator VAV1. To examine the role of ARF in the SHP-1 catalytic activity, the inventors performed phosphatase assays in the presence of ARF inhibitors. SHP-1 activity was significantly lower in the activated compared to inhibited NK cells (FIG. 8A; 47±10.1% vs. 100±2.8%, P≤0.001). Surprisingly, in the presence of ARF inhibitors the SHP-1 catalytic activity was significantly reduced in inhibited NK cells compared to untreated cells (for JAS: 72.8±5.9%, P≤0.001; for Y-27: 66.4±1.1%, P≤0.00001; and for both, JAS and Y-27: 67.4±1.7%, P≤0.00001). The level of activity was similar to that measured during activating interactions (P>0.06). No additive effect was observed under the inhibition by both, JAS and Y-27. Moreover, similar effects were detected following treatment of inhibited NK cells with CytD. These effects include increased binding of SHP-1 to β-actin following interaction of YTS-2DL1 cells with inhibitory 221-Cw4 cells (FIG. 8D), and significantly reduced phosphatase activity, relative to untreated cells (Cw4/untreated: 100% vs. Cw4/CytD: 55.4±8.4% P=0.02; FIG. 8E). These results indicate that ARF at the inhibitory NKIS controls SHP-1 catalytic activity, and further that actin turnover and myosin II activity are involved in the same regulatory mechanism acting on SHP-1.

The SHP-1 catalytic activity is determined by its conformational state, whereby the SHP-1 inactivated form is folded in an auto-inhibited (“closed”) conformation that is distributed in the cytoplasm. Upon NK cell inhibition SHP-1 acquires an “open” conformation induced by SHP-1 binding to the immunoreceptor tyrosine-based inhibitory motifs (ITIM) of the KIR receptors and release of the monovalent SH2 domains from phosphotyrosine interactions, and thus facilitation of SHP-1 phosphatase activity. The effect of ARF inhibition on the SHP-1 conformational state was determined using Fluorescence Resonance Energy Transfer (FRET) sensor, in which SHP-1 N′ and C′ terminal ends were tagged with YFP and CFP, respectively (FIGS. 8B and 9B). NK cells expressing YFP-SHP1-CFP were examined following interaction with 221-Cw4 or 221-Cw7 target cells expressing mCherry. Low FRET efficiency was measured in the inhibited compared to activated NK cells (FIG. 8C; 11.5±2.5% second panel vs. 25.3±2.9% top panel, P≤0.0009), suggesting that SHP-1 forms an inactive (folded) conformational state after NK cell activation. Further, the effect of ARF inhibition, using JAS, Y-27 or both, on SHP-1 conformation at the inhibitory NKIS resulted in significant increase of FRET efficiency, suggesting that ARF inhibition leads to transition of SHP-1 from “open” to “closed” (inactivated) conformation (FIG. 8C; untreated cells 11.5±2.5% compared to JAS 20.1±3.1%, P≤0.039, and Y-27 23.2±1.3%, P≤0.0001). Treatment with JAS and Y-27 during NK cell inhibition had no additive effect compared to individual treatments (FIG. 8C bottom panel; 23.8±1.5%, P≤0.0001). Furthermore, no changes in FRET efficiency were detected during NK cell activating interaction upon ARF inhibition by JAS (FIG. 9C; Cw7 untreated 25.3±2.9% compared to Cw7 JAS 24.2±3.2%, P>0.8). These results are consistent with the inventor's findings of the SHP-1/β-actin complex predominantly in the inhibitory and not activating NKIS (FIG. 2).

Additionally, analyzing the distribution of FRET signal at the NK:target contact site, relative to a non-synapse sites, indicated that following NK inhibitory interactions, the loss of signal due to formation of the SHP-1 open active conformation is specific to the NKIS, where the transmembrane inhibitory receptors are engaged. Nevertheless, no significant differences in the FRET signal were detected in the synapse (NKIS) versus the non-synapse regions in activated or inhibited NK cells treated with JAS (FIG. 9D).

To confirm a direct effect of ARF suppression on NK cell signaling, as opposed to possible alterations in the target cells, these experiments were repeated in an experimental system free of target cells, consisting of NK cells seeded over activating or inhibitory surfaces coated with anti-CD28 or anti-KIR2DL1 antibodies, respectively. In these experiments the NK cells were imaged at a single confocal plane of ˜1 μm at the contact interface of the NK cells with the antibody-coated slides. Focusing on this confocal plane enabled to detect SHP-1 conformational changes that occur specifically at the NKIS near the cytoplasmic tails of the receptors. The FRET data obtained in this system supported the results acquired with the NK:target cell system. Following KIR2DL1 engagement, SHP-1 adopted its active open conformation (6.1±1.5%, top panel, FIG. 9E), whereas ARF arrest using JAS significantly increased the FRET efficiency (18.5±1.3%, P=0.00001, second panel), to a level similar to that measured under activating conditions (14.6±1.6%, P=0.06, third panel; FIG. 9E). Moreover, measurements of YFP-SHP-1-CFP FRET in unstimulated cells seeded over uncoated surfaces demonstrated high FRET efficiency, as expected, which was similar to that measured in activated NK cells seeded over anti-CD28 surfaces (P=0.6; FIG. 9E, bottom panels). Overall, these data further support our findings that F-actin dynamics regulate the SHP-1 conformational state at the inhibitory NKIS, thereby governing its catalytic activity.

To eliminate the possibility that the pharmacological inhibitors such as JAS directly inhibit SHP-1 activity, and not via their effect on ARF, SHP-1 phosphatase activity was measured following addition of JAS directly to SHP-1 precipitates. YTS-2DL1 cells were incubated with inhibitory 221-Cw4 target cells, to induce SHP-1 activation, followed by immunoprecipitation of SHP-1 from cells lysates. SHP-1 precipitates were then treated with JAS, or left untreated, and SHP-1 phosphatase activity was measured. Direct treatment of SHP-1 with JAS did not inhibit SHP-1 activity, as compared to the untreated control sample (untreated: 100% vs. JAS: 96±12.3% P=0.77; FIG. 9A). These results indicate that JAS affects SHP-1 only in the cellular context by affecting actin dynamics.

To further determine the role of actin mechanotransduction in regulating SHP-1 conformation, a physiological approach was utilized to alter ARF dynamics by changes in the physical properties of NK cell microenvironment. Different tissues and tumors are characterized by unique degrees of surface rigidity (Tse J R, et al (2010) Current protocols in cell biology Chapter 10: Unit 10-16), which affect tumor progression and the immune response. While stiff surfaces were previously shown to arrest or retard actin flow, soft surfaces enable fast ARF (Hui K L et al (2015) Molecular biology of the cell 26: 685-695).

This system consists of seeding NK cells on substrates with different degrees of stiffness, which were extensively used to study a variety of biological processes, including, retrograde flow and cancer cell killing by immune cells (Hui K L et al (2015) Molecular biology of the cell 26: 685-695). A major advantage of this experimental setting is that it mimics the physiological micro-environment in which NK cells operate. This system enabled to directly control ARF velocity in NK cells.

YTS F-tractin cells were seeded over soft (1 kPa) vs. stiff (50 kPa) polyacrylamide hydrogels, and the velocity of ARF was determined at the contact site using live cell imaging and kymograph analysis. The results revealed that spreading of NK cell over soft surfaces (1 kPa) coated with inhibitory anti-KIR2DL1 antibody resulted in slow ARF, whereas coating of this hydrogel with activating anti-CD28 antibody resulted in fast ARF (KIR2DL1/1 kPa: 0.08±0.004 μm/sec vs. CD28/1 kPa: 0.25±0.009 μm/sec, P=0.00001; FIG. 8F). Nevertheless, NK cell spreading over stiff surfaces (50 kPa) resulted in total ARF arrest, in both inhibitory and activating settings (KIR2DL1/50 kPa: 0.004±0.0002 μm/sec, P=0.00001; CD28/50 kPa: 0.01±0.001 μm/sec, P=0.00001; FIG. 8F), confirming that changes in surface stiffness alter ARF in NK cells.

The changes in ARF velocity was correlated with the conformation of SHP-1 using FRET analysis of YFP-SHP-1-CFP at the contact site of the NK cells with the hydrogel surfaces. As expected, under inhibitory settings, soft surfaces enable SHP-1 to acquire its open active conformation, as detected by low FRET signal, whereas stiff surfaces led to SHP-1 assuming a closed inactive conformation at the inhibitory NKIS, as detected by significantly higher FRET efficiency (KIR2DL1/1 kPa: 6.6±2.1% vs. KIR2DL1/50 kPa: 15.4±2.8%, P=0.02; FIGS. 8G, 8H). Under activating settings, however, no changes in FRET efficiency were detected regardless of the stiffness of the surfaces (CD28/1 kPa: 18.2±2.6% vs. CD28/50 kPa: 16.5±2.7%, P=0.6; FIGS. 8G and 8H).

Overall, this data indicate that slow ARF, as observed at the inhibitory NKIS, supports the actin-SHP-1 interaction and SHP-1 activity, while ARF arrest maintains SHP-1 in the inactive state.

### Example 6

ARF Regulates SHP-1 Activity as Detected by Phosphorylation Level of its Natural Substrates

To further analyze the effect of ARF on SHP-1 activity at the inhibitory NKIS, its effect on the tyrosine phosphorylation profile of SHP-1 substrates VAV1 and PLCγ1/2 was determined. VAV1 activates small GTPase proteins of the Rho family, and is essential for actin reorganization and lytic granule polarization towards the target cells (Cella M, et al (2004) The Journal of experimental medicine 200: 817-823). YTS-2DL1 NK cells were incubated with 221-Cw4 or 221-Cw7 target cells, and treated with JAS following 5 min of incubation. Subsequently, the cell conjugates were stained with anti-pVAV1 (Y160) antibody to determine VAV1 activation status upon NK cell inhibition. As expected, significantly lower accumulation of phosphorylated VAV1 was detected at the inhibitory NKIS, relative to the activating one, as determined by measuring pVAV1 (Y160) synapse fluorescence intensity (FIG. 10A, left and middle top panels, and FIG. 10B, P=0.00001). Strikingly, suppression of F-actin dynamics by JAS dramatically increased VAV1 phosphorylation levels at the inhibitory NKIS (FIG. 10A, left bottom panel, and FIG. 10B P=0.00001), to levels resembling those detected at the activating NKIS with 221-Cw7 target cells (FIG. 10A, middle top panel, and FIG. 10B P=0.1). However, following NK cell activation, ARF inhibition by JAS led to a reduced accumulation of pVAV1 (Y160) at the NKIS (FIG. 10A, middle bottom panel, and FIG. 10B P=0.00001). These results imply that the enhanced activation of NK cells following JAS treatment are not due to the effect of JAS per-se, but are rather related to unique SHP-1 signaling occurring at the inhibitory NKIS.

To confirm these observations, SHP-1 knock-out YTS-2DL1 cells were prepared using CRISPR-Cas9 technology (YTS SHP-1^{-/-} cells; FIG. 11A). As predicted, knock-out of SHP-1 resulted in a significant increase in the accumulation of phospo-VAV1 (Y160) at the inhibitory NKIS, relative to WT NK cells that express endogenous SHP-1 (FIG. 10A, right vs. left top panels, and FIG. 10B, P=0.00001). Treatment of SHP-1 knock-out cells with JAS did not lead to a further increase in VAV1 phosphorylation, but rather, resulted in decreased accumulation of pVAV1 (Y160) at the inhibitory NKIS (FIG. 10A, right bottom panel, and 10B, P=0.008). These data indicate that ARF arrest by JAS at the inhibitory NKIS increases NK cell activation specifically via inactivation of SHP-1, and not by directly mediating any activating signals.

To further determine the role of ARF in regulating SHP-1 activity at the inhibitory NKIS, primary KIR2DL1⁺ cells were isolated from human donors. Analysis of synapse fluorescence intensity demonstrated the lack of accumulation of pVAV1 (Y160) at the inhibitory NKIS with 221-Cw4 cells, whereas JAS treatment resulted in a significantly higher phospho-VAV1 accumulation (FIG. 10C, P=0.00001), reaching the levels detected at the activating NKIS with 721.221 cells (FIG. 10C, P=0.2). In addition, IP experiments using anti-phosphotyrosine (pTy) demonstrated that ARF suppression increased VAV1 phosphorylation levels during the NK cell inhibitory response (FIG. 10D), confirming the imaging data. These experiments demonstrate that ARF regulates SHP-1 activity.

To determine the influence of actin dynamics on additional key signaling events in NK cells, the effect of ARF suppression on PLCγ1/2 phosphorylation was examined. These proteins are responsible for the release of Ca²⁺ from the endoplasmic reticulum (Joseph et al, 2014), crucial for NK cell effector functions, namely cytokine production and cytotoxicity (Caraux et al, 2006; Tassi et al, 2005). Following inhibitory receptor engagement, SHP-1 dephosphorylates PLCγ isoforms, resulting in NK cell inhibition (Matalon et al, 2016). YTS-2DL1 cells were conjugated with 221-Cw4 or Cw7 target cells, and treated with JAS following 5 min incubation. Immunoprecipitates using anti-pTy were immunoblotted with anti-PLCγ1, revealing elevated phosphorylation levels at the inhibitory NKIS following ARF arrest (FIG. 10E). Nevertheless, following NK cell activation, no effect was detected, as PLCγ1 was highly phosphorylated regardless of ARF suppression (FIG. 10E). Reciprocal IPs using anti-PLCγ1, and IB with anti-pPLCγ1 (Y783) also demonstrated elevated PLCγ1 phosphorylation levels following ARF suppression specifically following NK cell inhibition (FIG. 11B), but not activation (FIG. 11C). Furthermore, analyzing the effect of ARF on the phosphorylation of the PLCγ2 isoform, via both biochemical (FIG. 10F) and imaging (FIG. 11D) approaches, revealed a similar trend.

Finally, the effect of ARF inhibition on the phosphorylation of the SHP-1 substrate, VAV1, was also determined using the polyacrylamide hydrogel system. YTS-2DL1 cells were seeded over soft (1 kPa) versus stiff (50 kPa) polyacrylamide hydrogels coated with either anti-CD28 antibody, to induce NK cell activation, or with a combination of anti-CD28 and anti-KIR2DL1 antibodies, to induce NK cell inhibition. Uncoated hydrogels were used as a control to determine the basal phosphorylation of VAV1. Following NK cell activation, cells were stained with anti-pVAV1 (Y160) antibody, and the fluorescence intensity of phosphorylated VAV1 was measured at the contact site of the NK cells with the hydrogel surfaces. As expected, significantly lower VAV1 phosphorylation was detected at inhibitory contact sites, relative to the activating one (FIG. 11E; P≤0.00005). However, spreading of NK cells over stiff surfaces, which suppress F-actin dynamics, significantly increased VAV1 phosphorylation levels when NK cells were seeded over anti-CD28 and KIR2DL1 coated hydrogel surfaces (FIG. 11E; P=0.00001).

Altogether, these results suggest that ARF suppression leads to reduced activity of SHP-1, as indicated by the enhanced VAV1 and PLCγ1/2 phosphorylation patterns. These data are consistent with the FRET and PTP activity data, indicating the conversion of SHP-1 conformation to an inactive form following suppression of ARF. Overall, these results strongly suggest that ARF regulates NK cell signaling by controlling SHP-1 catalytic activity.

### Example 7

ARF Regulates NK Cell Function During the Inhibitory Response

The inventors examined whether the ARF-mediated regulation of SHP-1 activity further dictates the activation status and functional outcome of NK cells. Since PLCγ1/2 control intracellular calcium concentrations essential for NK cellular activation, Ca²⁺ flux was measured in KIR2DL1 expressing YTS or primary NK cells following ARF inhibition. NK cells were incubated with target cells and were then treated with JAS and monitored for Ca²⁺ levels. While calcium mobilization was remarkably lower during the inhibitory vs. activating response, JAS treatment inverted this trend, resulting in elevated Ca²⁺ flux during NK cell inhibition (FIGS. 12A, 12B). These results demonstrated that the F-actin network dynamics controls downstream NK cell responses. During NK cell activation, however, ARF suppression by JAS decreased intracellular Ca²⁺ concentration (FIG. 11F), in correlation with a reduced accumulation of F-actin at the activating NKIS (FIG. 11G). These results demonstrate that the suppression of F-actin dynamics upregulates downstream NK cell responses specifically following NK cell inhibition.

Next, the effect of ARF inhibition on NK cell cytotoxicity was directly examined by observing the NK cell-mediated killing of target cells using real-time live-cell imaging. Activating 221-Cw7 and inhibitory 221-Cw4 target cells were labelled with the vital dye calcein-AM, which diffuses across the plasma membrane following target cell lysis (Guldevall K, et al (2010) PloS one 5: e15453). The leakage of calcein from dying cells, and the resultant fluorescence loss, enables the distinction between dead and live target cells, allowing the quantification of NK cell killing efficiency. To control for the effect of bleaching per se on fluorescence loss, calcein-labeled target cells were seeded alone, in the absence of NK cells, and monitored fluorescence intensities (FIG. 12E). Treatment of target cells alone with JAS had no additional effect on fluorescence intensity, indicating that JAS does not induce spontaneous target cell death (FIG. 12E). In parallel, target cells were plated over slides, followed by seeding of YTS-2DL1 cells. Following 5 min of incubation, the cells were treated with JAS or left untreated, and fluorescence losses were monitored over time (data not shown). As expected, the incubation of NK cells with inhibitory 221-Cw4 cells did not result in target cell death (FIG. 12E), whereas NK cell incubation with activating 221-Cw7 cells led to significantly higher target cell lysis (FIG. 12E; P=0.01). Strikingly, ARF suppression in NK cells conjugated with 221-Cw4 target cells significantly increased the cytotoxicity against inhibitory target cells (FIG. 12E; P=0.01), relatively to that of untreated cells (FIG. 12E), reaching the cytotoxicity levels of activating target cells (FIG. 12E; P=0.2).

To validate these results, the functional consequences of ARF suppression were further evaluated by measuring NK cell degranulation following interaction with inhibitory or activating target cells. KIR2DL1⁺ YTS or primary NK cells were incubated with mCherry-expressing 221-Cw4 or Cw7 target cells followed by ARF suppression (for mCherry expression levels, see FIG. 11H). A significant elevation in the secretion of cytolytic granules by YTS cells was observed in response to ARF arrest relative to untreated cells following interaction with inhibitory target cells (27.4%±4.7 vs. 5.8%±2.6, P=0.02; FIG. 12C and FIG. 11I). Interestingly, a more pronounced trend was observed with primary isolated NK-2DL1 cells (42.6%±9.7, relative to untreated cells 10%±3.3, P=0.02; FIG. 12D and FIG. 11J). The activity resembled the degranulation level during an activating response (YTS: 20.1%±4.2, P=0.3; pNK: 33.4%±1.5, P=0.4).

These findings suggest that ARF plays a key role in dictating the activation threshold and the functional outcome of NK cells, and suggest a novel strategy for regulating NK cell cytotoxicity by controlling actin dynamics. A schematic layout of this understanding is illustrated in FIG. 13. Furthermore, these findings have led the inventors to propose a novel strategy for controlling NK cell function by manipulating actin dynamics. Feasibility studies in support of this strategy are currently ongoing, as detailed in the following EXAMPLES 8-9.

### Example 8

Systematic Screening of Small Molecule Drug Libraries for Novel ARF Inhibitors Having the Potential to Modulate NK Cell Killing Efficiency

The inventors are presently focusing on small molecule libraries of known and unknown actin modulators and analyze their effect on ARF in NK cells. Libraries of known compounds are available from companies such as Merck-Millipore and Enzo. These libraries are screened for their effects on the organization and dynamics of actomyosin network and on responsiveness of NK cells.

Specifically, this approach is divided into three main stages. (i) Small molecules libraries are screened by a high-throughput high-content microscopy system. NK cells expressing a fluorescently tagged F-tractin are automatically exposed to the library compounds using a robotic platform, and are seeded over activating vs. inhibitory fluorescently labeled target cells. Upon imaging acquisition of NK-target conjugates, data analysis aims at identifying specific compounds that alter both F-actin accumulation at the NKIS and target cell killing Within this framework, the inventors have shown that ARF inhibition by JAS results in significant increase of F-actin accumulation at the inhibitory NKIS (FIG. 14 upper panel; P≤0.00001), and a decrease at the activating NKIS (FIG. 14 lower panel; P≤0.01). These findings indicate that F-actin accumulation and target cell lysis may serve as markers (or primary parameters) for actomyosin dynamics, meaning that small molecules that alter these two parameters may be considered as positive hits or candidates for further validation. (ii) The small molecule candidates from stage (i) are examined for their effects on ARF. The NK cells are treated with candidate molecules and are plated over the microchips pre-coated with inhibitory and activating antibodies. Fluorescently tagged F-tractin and NMHC-IIA are expressed in NK cells and the influences of small molecules in slowing or accelerating ARF are evaluated. The potency of specific molecule to regulate actomyosin network dynamics is evaluated by calculating EC50 values (50% of maximal effective concentration). (iii) Responsive vs. hyporesponsive NK cells or activated vs. inhibited NK cells are pre-treated with candidate ARF modulators selected according to the above, wherein actomyosin dynamics is compared to untreated cells, and their cytotoxic capability is analyzed, e.g. EXAMPLE 7.

Ultimately, the above platform serves for identifying potential novel ARF modulators that transduce hyporesponsive or inhibited NK cells into responsive or activated, and vice versa.

The following EXAMPLE 9 describes how the inventors determine therapeutic relevance of these novel ARF modulators for reshaping NK cell immune response in-vivo.

### Example 9

Control NK Cell Responsiveness In-Vivo Using Nanoparticle Targeted Drug Delivery System Containing ARF Modulators

The biological relevance of the ARF-related mechanisms in in-vivo modulation of NK cell inhibition vs. activation or responsiveness vs. hyporesponsiveness is currently under study using MHC^{-/-} mice that give rise to anergic or hyporesponsiveness NK cells and the B6 wt mice that produce responsive NK cells. The purpose of using this mouse model is to achieve fine tuning of the underlying molecular mechanisms, which cannot be achieved in the human research system. Within this framework, the inventors implement liposomal nanoparticle (NP) based delivery system. NPs that are selectively targeted to specific cell types may provide selective delivery of therapeutic agents with significantly reduced side effects. Liposomal NPs are pharmaceutically proven delivery vehicles that can encapsulate a therapeutic agent and further display ligands that target cell-surface receptors. The inventors have previous experience with this procedure and a working protocol for applying it in T-lymphocytes.

Preparation of the liposomal NP is schematically illustrated in FIG. 15A. To enable NP detection DPPE labeled with Rhodamine red (DPPE-PE) is incorporated into the lipid mixture (FIG. 15B). The protocol for NP preparation is detailed in the Experimental procedures, particle diameters and surface charges (zeta potential) are measured during the various stages of their preparation. Selectivity of anti-LFA1 coated NPs was already evaluated in showing exclusive NP uptake by LFA-1 expressing peripheral blood lymphocytes (PBLs) but not K562 cells lacking LFA-1 expression (FIG. 15C). Further, uptake of NPs coated with HA (CD44 ligand) by NK cells was also evaluated, this experiment demonstrated the ability of this system to efficiently transduce primary NK cells (FIG. 15D).

The ultimate goal is to implement the above NPs in in vivo targeting of NK cells. These experiments are divided into three stages: (i) anti-NKp46 antibody coated NPs are loaded with the candidate ARF modulators and their uptake by the NK cells is tested in-vitro by confocal microscope and FACS analyses. Functional consequences of delivering various ARF/signaling modulators are determined by tracking actomysin dynamics and measuring NK cell cytotoxicity. (ii) To investigate the ability of NK targeted NPs to modulate NK cell responsiveness in-vivo, NPs is administered by intravenous injection to MHC^{-/-} and wt mice. ARF is followed in isolated NK cells after 72 hours, and the ability of transducing hyporesponsiveness NK cells into responsive (and vice versa) or inhibited NK cell into activated cells (and vice versa) is determined by measuring cytolytic activity to cancer cells in-vitro. (iii) To determine therapeutic feasibility of NPs in-vivo, MHC^{-/-} and wt mice are inoculated with cancer cells e.g. melanoma cell line. NK cell mediated rejection of tumor graft is measured in the MHC^{-/-} and wt mice by monitoring changes in tumor size and volume, using a computerized tomography Maestro providing in-vivo fluorescence imaging of a whole body. This device and method can provide time-based kinetic images of the fluorescent NPs, thereby enabling monitoring the distribution and recruitment of NK cells at the tumor. Mean survival time (MST) of mice treated with loaded NPs vs. empty NPs is further estimated.

1-19. (canceled)

20. A method for modulating hematopoietic cell activation, the method comprising contacting said cell with a modulatory effective amount of a modulator or any vehicle, matrix, nano- or micro-particle, or composition comprising the same, wherein said modulator is characterized in that it modulates ARF in a cell.

21. The method according to claim 20, wherein said hematopoietic cell is a lymphocyte cell being at least one of an NK cell, a T cell and a B cell forming an inhibitory or activating IS.

22. The method according to claim 21, wherein said lymphocyte cell is an NK cell forming an inhibitory or activating NKIS.

23. The method according to claim 22, wherein said modulator is an ARF inhibitor that disturbs ARF in said NK cell, and wherein said modulation activates NK cells in an inhibitory NKIS.

24. The method according to claim 23, wherein disruption of ARF results in at least one of formation of a complex comprising β-actin and at least one PTP, change in the conformation of at least one PTP and change in the catalytic activity of said PTP in said NK cell.

25. The method according to claim 24, wherein said at least one PTP is SHP and wherein said β-actin:SHP complex induces a change in the SHP conformation and catalytic activity in said NK cell.

26. The method according to claim 23, wherein said SHP is at least one of SHP-1 and SHP-2.

27. The method according to claim 23, wherein activation of NK cells results in an increase in at least one of intracellular Ca²⁺ flux, and secretion of cytolytic granules in said NK cell.

28. The method according to claim 23, wherein said ARF inhibitor is at least one of an inhibitor of actin depolymerization, an F-actin stabilizer and an inhibitor or at least one of myosinIIA phosphorylation and activity.

29. The method according to claim 28, wherein said ARF inhibitor is any one of JAS, Y-27632 and CytD or any derivatives or any combinations thereof.

30. The method according to claim 20, for modulating hematopoietic cell activation in a subject in need thereof, said method comprises administering to said subject a modulatory effective amount of a modulator that modulates at least one of actin and myosin ARF in a cell, or of any vehicle, matrix, nano- or micro-particle, or composition comprising the same, wherein said subject is a mammalian subject suffering of an immune-related disorder, optionally, said immune-related disorder is at least one of a viral infection, cancer, a proliferative disorder, a graft versus host disease, an inflammatory disorder, an immune-cell mediated disorder and an autoimmune disorder.

31-32. (canceled)

33. The method according to claim 20, for treating, preventing, inhibiting, reducing, eliminating, protecting or delaying the onset of an immune-related disorder in a subject in need thereof, said method comprises administering to said subject a therapeutically effective amount of at least one modulator that modulates at least one of actin and myosin ARF in a cell, or of any vehicle, matrix, nano- or micro-particle, or composition comprising the same.

34. The method according to claim 33, wherein said hematopoietic cell is a lymphocyte cell being at least one of an NK cell, a T cell and a B cell forming an inhibitory or activating IS, optionally, wherein said lymphocyte cell is a NK cell forming an inhibitory or activating NKIS.

35. (canceled)

36. The method according to claim 35, wherein said modulator is an ARF inhibitor that disturbs ARF in said NK cell, thereby activating NK cells in an inhibitory NKIS, optionally, at least one of:
(a) wherein disruption of ARF results in at least one of formation of a complex comprising β-actin and at least one PTP, change in the conformation and catalytic activity of said PTP in said NK cell, optionally, said at least one PTP is SHP and wherein said β-actin:SHP complex induces a change in the SHP conformation and catalytic activity in said NK cell, said SHP is at least one of SHP-1 and SHP-2; and
(b) wherein activation of NK cells results in an increase in at least one of intracellular Ca2+ flux, and secretion of cytolytic granules in said NK cell.

37-40. (canceled)

41. The method according to claim 36, wherein said ARF inhibitor is at least one of an inhibitor of actin depolymerization, an F-actin stabilizer, and an inhibitor of at least one of myosinIIA phosphorylation and activity.

42. The method according to claim 41, wherein said ARF inhibitor is any one of JAS, Y-27632 and CytD, or any derivatives or any combinations thereof.

43. The method according to claim 33, wherein said immune-related disorder is at least one of a viral infection, cancer, a proliferative disorder, a graft versus host disease, an inflammatory disorder, an immune-cell mediated disorder and an autoimmune disorder.

44. A modulator of lymphocyte cell activation or any vehicle, matrix, nano- or micro-particle, or composition comprising the same, wherein said modulator is characterized in that it modulates at least one of actin and myosin retrograde flow (ARF) in a hematopoietic cell.

45. The modulator according to claim 44, wherein said hematopoietic is a lymphocyte cell being at least one of an NK cell, a T cell and a B cell, forming an inhibitory or activating IS, optionally, wherein said lymphocyte is an NK cell forming an inhibitory or activating NKIS, and wherein said modulator is an ARF inhibitor that disturbs ARF in said NK cell, said modulation activates NK cells in an inhibitory NKIS.

46-49. (canceled)

50. A method for screening for a modulator of NK cell activation, the method comprising the steps of:
(a) contacting said NK cell with at least one of activating or inhibitory target cell or with a solid support coated with at least one of activating or inhibitory molecules;
(b) contacting said NK cell with at least one of activating or inhibitory target cell/or with a solid support coated with at least one of activating or inhibitory molecules in the presence of a candidate modulator compound; and
(c) determining at least one of:
(i) accumulation of at least one of F-actin and myosin in said NK cells of (a) and of (b);
(ii) at least one of F-actin and myosin dynamics in said NK cells of (a) and of (b); and
(iii) target cell lysis by said NK cells of (a) and of (b);
wherein a change in at least one of accumulation of at least one of F-actin and myosin as determined in step (c i) and at least one of F-actin and myosin dynamics as determined in step (c ii) and target cell lysis as determined in step (cii) in the presence of said candidate compound of (b) as compared to the absence of said compound of (a) indicates that said candidate compound modulates NK cell activation.
